(19) United States
(12) Patent Application Publication
(10) Pub. No.: US 2006/0189622 A1
(43) Pub. Date: Aug. 24, 2006

(54) Pyridazinone and triazinone compounds and use thereof as pharmaceutical preparations

(76) Inventors: Satoshi Nagato, Chiba (JP); Koki Kawano, Ibaraki (JP); Koichi Ito, Chiba (JP); Yoshihiko Norimine, Ibaraki (JP); Kohshi Ueno, Ibaraki (JP); Takahisa Hanada, Ibaraki (JP); Hiroyuki Amino, Ibaraki (JP); Makoto Ogo, Ibaraki (JP); Shinji Hatakeyama, Ibaraki (JP); Masataka Ueno, Ibaraki (JP); Anthony John Groom, Wiltshire (GB); Leanne Rivers, Kent (GB); Terence Smith, Cambridgeshire (GB)

Correspondence Address: BIRCH STEWART KOLASCH & BIRCH, PO BOX 747, FALLS CHURCH, VA, 22040-0747, US

(21) Appl. No.: 11/408,078

(22) Filed: Apr. 21, 2006

Related U.S. Application Data
(63) Continuation of application No. 10/380,783, filed on Mar. 18, 2003, now abandoned.

(30) Foreign Application Priority Data
Sep. 18, 2000 (JP) 2000-282636
Sep. 22, 2000 (JP) 2000-289412
Nov. 9, 2000 (JP) 2000-342614
Feb. 5, 2001 (GB) 0102822.4
Feb. 5, 2001 (GB) 0102824.0

(51) Int. Cl. A61K 31/506; A61K 31/501; A61K 31/50; C07D 409/02; C07D 403/02; C07D 405/02

(52) U.S. Cl. 514/252.01; 514/252.02; 514/252.03; 544/238; 544/239

(57) ABSTRACT

The present invention provides a novel compound exhibiting an excellent inhibitory action on AMPA receptor and/or kainate receptor. That is, it provides a compound represented by the following formula, a salt thereof or a hydrate of them. 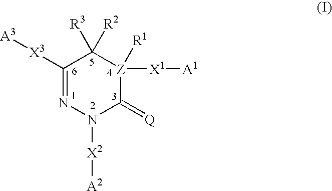
In the formula, A¹, A² and A³ are independent of each other and each represents a C₃₋₈ cycloalkyl group, a C₃₋₈ cycloalkenyl group, a 5- to 14-membered non-aromatic heterocyclic group, a C₆₋₁₄ aromatic hydrocarbon cyclic group or a 5- to 14-membered aromatic heterocyclic group, each of which may be substituted; Q represents O, S or NH; Z represents C or N; X¹, X² and X³ are independent of each other and each represents a single bond, an optionally substituted C₁₋₆ alkylene group, an optionally substituted C₂₋₆ alkenylene group, an optionally substituted C₂₋₆ alkynylene group, —NH—, —O—, —NHCO—, —CONH—, —SO₀₋₂—, etc.; R¹ and R² are independent of each other and each represents a hydrogen atom or an optionally substituted C₁₋₆ alkyl group, or R¹ and R² may be bound together such that CR²-ZR¹ forms C═C; and R³ represents a hydrogen atom or an optionally substituted C₁₋₆ alkyl group etc., or may be bound to any atom in A¹ or A³ to form, together with the atom, an optionally substituted C₅₋₈ hydrocarbon ring or an optionally substituted 5- to 8-membered heterocyclic ring.

### CROSS-REFERENCE TO RELATED APPLICATIONS

This application is a 37 C.F.R. § 1.53(b) continuation of U.S. application Ser. No. 10/380,783 filed Mar. 18, 2003, which is the National Phase of PCT International Application No. PCT/JP01/08058 filed Sep. 17, 2001, which claims priority on Japanese Application Nos. 2000-282636 filed Sep. 18, 2000; 2000-289412 filed Sep. 22, 2000; and 2000-342614 filed Nov. 9, 2000, and United Kingdom Application Nos. 0102822.4 and 0102824.0, both filed Feb. 5, 2001. The entire contents of each of these applications is hereby incorporated by reference.

### FIELD OF THE INVENTION

The present invention relates to a novel compound, a salt thereof and a hydrate of them, to methods for manufacturing the same, and to use thereof as pharmaceutical preparations. More specifically, it relates to pyridazinone and triazinone compounds useful as non-NMDA receptor inhibitors, particularly as AMPA receptor inhibitors.

### PRIOR ART

Glutamate and aspartate are important amino acids which participate in nerve functions such as recognition, memory, movement, respiration, cardiovascular adjustment and sensation and are called excitatory neurotransmitters as well. In the expression of their physiological activities, an interaction with a specific receptor is important and, generally, two types of receptors—an ion channel type and a G-protein coupled type —have been known. The former is further classified into N-methyl-D-aspartate (NMDA) receptor, α-amino-3-hydroxy-5-methyl-4-isoxazole propionic acid (AMPA) receptor, kainate receptor, etc. On the other hand, the amino acid as an excitatory neurotransmitter has been known to induce neurotoxicity by, for example, abnormal excitation of central nerves. It has been noted that the said toxicity is as serious as being accompanied by the death of nerve cells causing various nervous diseases. Main nervous diseases which have been known are cerebral ischemia, head injury, spinal cord injury, Alzheimer's disease, Parkinson's disease, amyotrophic lateral sclerosis (ALS), Huntington's chorea, AIDS nervous disturbance, epilepsy, neurodegenaration observed after the state of hypoxia, mental disorder, mobility disturbance, pain, spasticity, nervous disturbance by toxin in food, various neurodegenerative diseases, various mental diseases, chronic pain, migraine, cancer pain and pain caused by diabetic nervous disturbance. They are serious diseases where many mechanisms of onset, etc. have not been clarified yet and pharmaceutical agents which are effective for the therapy have not been found yet but it is believed that they are closely related to excessive release/accumulation of excitatory neurotransmitters, changes in expressing pattern of receptors, etc. For example, it has been reported that glutamate concentration in cerebrospinal fluid and plasma increases in stroke, cerebral ischemia, head injury and spinal cord injury (Castillo, J., Dazalos, A. and Noya, M., Lancet, 1997, 346: 79-83; etc.). There is a report that neuropathy occurs when glutamate, NMDA, AMPA, kainate, etc. are excessively applied to nerve cells (Meldrum, B., Brain Res. Reviews, 18, 293, 1993). There are reports that, in Alzheimer's disease, β-amyloid protein enhances the neurotoxicity of glutamate and that it promotes the release of glutamate (Arias, C., Arrieta, I. and Tapia, R., J. Neurosci. Res., 1995, 41: 561-566; etc.). In the case of Parkinson's disease, there are reports that L-dopa hydroxide activates the AMPA receptor (Cha, J. J., et. al., Neurosci. Lett., 1991, 132: 55-58) and enhances the neurotoxicity (Olney, J. W., et. al., 1990, 108: 269-272; Rosenberg, P. A., et. al., Proc. Natl. Acad. Sci. USA, 1991, 88: 4865-4869). There is another report that L-dopa promotes the generation of free radicals resulting in a rise of oxidative stress (Smith, T. S., et. al., Neuroreport, 1994, 5: 1009-1011). In the case of Huntington's chorea, it is reported that a substance which inhibits the release of glutamate is effective in improving the symptoms. In the case of ALS, there are many reports showing the participation of glutamate in its pathology. There are some cases where the AIDS patients suffer from recognition nerve function deficiency and, even in such a nerve disease, participation of glutamate is suggested. For example, it is reported that gp 120 which is a glycoprotein in an envelope of HIV virus suppresses the uptake of glutamate by astrocytes (Dreyer, E. B., Eur. J. Neurosci., 1995, 7: 2502-2507; Ushijima, H., et. al., Eur. J. Neurosci., 1995, 7: 1353-1359) while a substance which inhibits the release of glutamate suppresses the neurodegeneration by gp 120 (Sindou, P., et. al., J. Neurosci. 1994, 126: 133-137; Muller, W. E. G., et. al., Eur. J. Pharmacol. Molec. Pharmacol., 1992, 226: 209-214; Lipton, S. A., Neurology, 1992, 42: 1403-1405). With regard to allergic encephalomyelitis, there is a report that, in the mice where the said inflammation takes place, enzyme which decomposes glutamate incorporated from outside of cells is deficient (Hardin-Pouzet, H., Glia., 1997, 20: 79-85). Olivopontocerebellar atrophy is a disease which is sometimes combined with Parkinson's disease and an antibody to GluR2 which is a subunit constituting the AMPA receptor has been found (Gahring, L. C., Neurology, 1997, 48: 494-500) and the relation between olivopontocerebellar atrophy and AMPA receptor is suggested. With regard to a report for epilepsy, it is reported that, in the mice which are unable to construct the GluR2 in AMPA receptor, Ca²⁺ permeability of the AMPA receptor increases whereby it is apt to cause a sudden onset resulting in death (Brusa, R., Science, 1995, 270: 1677-1680). Besides the above, it is reported that NBQX (2,3-dihydroxy-6-nitro-7-sulfamoylbenz[f]-quinoxaline; Sheardown, et al., Science, 247, 571, 1990) and other inhibiting compounds to AMPA receptors have antianxiety and anticonvulsant action (J. Pharmacol. Exp. Ther., 260, 742, 1992; Pharmacol. Biochem. Behavior, 1998, 60: 119-124) and there are also reports for the connection of AMPA receptor/kainate receptor with urinary disturbance, drug abuse, pain, etc. (J. Pharmacol. Exp. Ther., 280, 894-904, 1997; Neuroscience Letters, 268: 127-130, 1999).

It can be expected that the substances showing an antagonistic action to excitatory neurotransmitter receptors are useful for the therapy of the above-mentioned nerve diseases. At present, the usefulness of the substances having an antagonistic action to non-NMDA receptors such as AMPA receptor and kainate receptor is particularly expected. For example, it is reported that inhibitors of the interaction of glutamate with the AMPA and/or kainate receptor complex are useful in treating demyelinating disorders such as encephalitis, acute disseminated encephalomyelitis, acute demyelinating polyneuropathy (Guillain Barre syndrome), chronic inflammatory demyelinating polyneuropathy, multiple sclerosis, Marchifava-Bignami disease, central pontine myelinolysis, Devic syndrome, Balo disease, HIV- or HTLV-myelopathy, progressive multifocal leucoencephalopathy, a secondary demyelinating disorder; for example, CNS lupus erythematodes, polyarteritis nodosa, Sjogren syndrome, sarcoidosis, isolated cerebral vasculitis, etc. as secondary demyelinating disorders, etc. in WO00/01376. With regard to the compound having an inhibitory action to AMPA receptor and kainate receptor, there are reports for the following compounds for example.
(1) Competitive AMPA receptor-inhibiting compounds represented by the following formula. 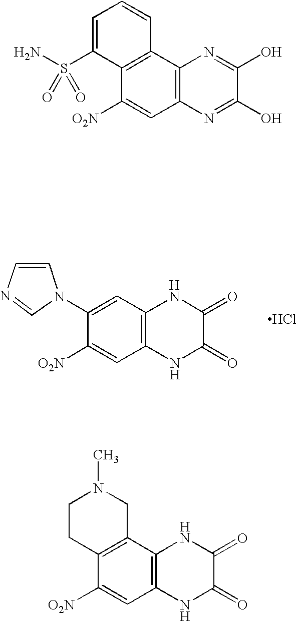
(2) Non-competitive AMPA receptor-inhibiting compounds represented by the following formula. 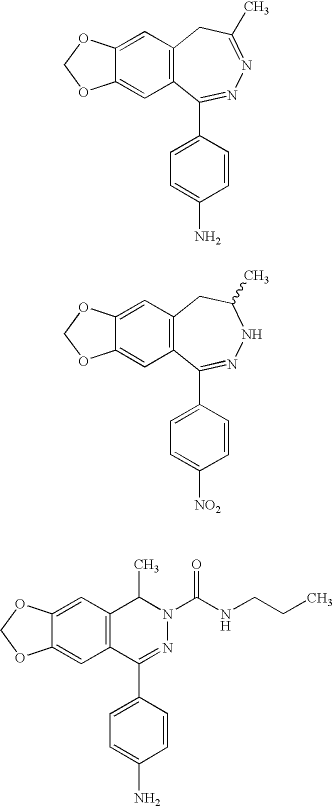
(3) Besides the above, there are reports on competitive AMPA receptor-inhibiting compounds having a quinoxalinedione skeleton in WO 94/25469, WO 96/10023, U.S. Pat. No. 5,356,902, etc. and there are reports on non-competitive AMPA receptor-inhibiting compounds in WO 95/01357, WO 97/28135, WO 97/28163, WO 97/43276, WO 97/34878, WO 98/38173, EP 802195, DE 19643037, etc. In WO97/17970, there is a report on a pyridothiazine derivative having an inhibitory action on the neurocytotoxicity of kainate, which is based on non-competitive antagonism against AMPA receptor response. In WO00/27851, there is a report on a condensed pyridazinone derivative having an enhancing action on memorization, which is based on enhancing action of NMDA and inhibitory action on AMPA. In WO00/47567, there is a report on a compound as a heterodiazinone derivative having antagonism against non-NMDA receptor, which is represented by the formula: 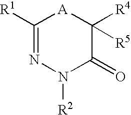
wherein A represents O, S or NR³ (wherein R³ is a hydrogen atom or a lower alkyl group); R¹ and R² are independent of each other and each represents an optionally substituted (hetero)aryl group; and R⁴ and R⁵ independently represents a hydrogen, hydroxyl group, halogen, cyano, nitro, lower alkyl,(hetero)aryl group, etc.

In WO99/10331, WO99/10332 and WO00/24719, there is a report on a pyridazinone compound as cyclooxygenase-2 inhibitor etc., which is represented by the following formula, or a salt, ester or prodrug thereof: 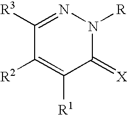
wherein X represents O, S, etc.; R represents an aryl group etc.; and at least one of R¹, R² and R³ represents a phenyl group substituted with a specific group etc., while the two other groups represent an aryl group etc. In WO99/25697, 99/44995 and WO00/50408, there is a report on a pyridazinone derivative as an inhibitor of production of interleukin-1β. In WO00/09488, there is a report on a pyridazinone derivative having an inhibitory action on cell adhesion. In WO97/07104, EP0860435, EP0963978, WO00/34249, U.S. Pat. No. 6,107,250, JP-A 5-25164, DE4423934, etc., there is also a report on a pyridazinone derivative having an antimicrobial activity and a heribicidal action for use in agrochemicals, but the relationship thereof with AMPA receptor/kainate receptor is not described therein and not known. There are some reports on use of triazinone compounds as agrochemicals, but the relationship thereof with AMPA receptor/kainate receptor is not described and not known. The relationship of a pyridazin-3-one derivative having cyclic substituent groups at the 2-, 4- and 6-positions, and a 1,2,4-triazin-3-one derivative, with AMPA receptor/kainate receptor is not known either.

It is desired to provide a compound which exhibits an excellent inhibitory action on AMPA receptor and/or kainate receptor, is highly useful as a pharmaceutical preparation effectively acting in clinical. Thus, an object of the present invention is to investigate and find a compound which inhibits AMPA receptor and/or kainate receptor which suppresses the neurotoxicity of excitatory neurotransmitters and achieves an excellent neuroprotective action as pharmaceutical agents being useful as an agent for treating, preventing or improving various nerve diseases.

### DISCLOSURE OF THE INVENTION

Under such circumstances, the present inventors have carried out an intensive study. As a result, they have succeeded for the first time in synthesizing a compound (Compound (I)) represented by the following formula, a salt thereof or a hydrate of them, and have found an excellent method for producing the compound, a salt thereof or a hydrate of them. Further, surprisingly, they have found that the above compound (I), a salt thereof or a hydrate of them shows an excellent AMPA receptor and/or kainate receptor antagonism, whereupon the present invention has been accomplished.

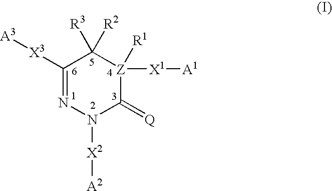

In the formula, A¹, A² and A³ are independent of each other and each represents a C₃₋₈ cycloalkyl group, a C₃₋₈ cycloalkenyl group, a 5- to 14-membered non-aromatic heterocyclic group, a C₆₋₁₄ aromatic hydrocarbon cyclic group or a 5- to 14-membered aromatic heterocyclic group, each of which may be substituted; Q represents O, S or NH; Z represents C or N; X¹, X² and X³ are independent of each other and each represents a single bond, an optionally substituted C₁₋₆ alkylene group, an optionally substituted C₂₋₆ alkenylene group, an optionally substituted C₂₋₆ alkynylene group, —NH—, O, —N(R⁴)CO—, —CON(R⁵)—, —N(R⁶)CH₂—, —CH₂N(R⁷)—, —CH₂CO—, —COCH₂—, —N(R⁸)SO₀₋₂—, —SO₀₋₂N(R⁹)—CH₂SO₀₋₂—, —SO₀₋₂CH₂—, —CH₂O—, —OCH₂—, —N(R¹⁰)CON(R¹¹)—, —N(R¹²)CS—N(R¹³)— or —SO₀₋₂— (wherein R⁴, R⁵, R₆, R⁷, R⁸, R⁹, R¹⁰, R¹¹, R¹² and R¹³ are independent of each other and each represents a hydrogen atom, a C₁₋₆ alkyl group or a C₁₋₆ alkoxy group; R¹ and R² are independent of each other and each represents a hydrogen atom, an optionally substituted C₁₋₆ alkyl group, an optionally substituted C₂₋₆ alkenyl group or an optionally substituted C₂₋₆ alkynyl group, or R¹ and R² may be bound to each other such that CR²-ZR¹ forms a carbon-carbon double bond represented by C═C (provided that when Z is N, R¹ represents a lone pair); R³ represents a hydrogen atom, an optionally substituted C₂₋₆ alkyl group, an optionally substituted C₂₋₆ alkenyl group or an optionally substituted C₂₋₆ alkynyl group, or may be bound to any atom in A¹ or A³ to form, together with the atom, an optionally substituted C₅₋₈ hydrocarbon ring or an optionally substituted 5- to 8-membered heterocyclic ring (provided that (1) when Z is N; each of X¹, X² and X³ is a single bond; and each of A¹, A² and A³ is a phenyl group, (2) when Z is N; each of X¹, X² and X³ is a single bond; A¹ is an o,p-dimethylphenyl group; A² is an o-methylphenyl group; and A³ is a phenyl group, or (3) when Z is N; each of X¹, X² and X³ is a single bond; A¹ is an o-methylphenyl group; A² is a p-methoxyphenyl group; and A³ is a phenyl group, at least one of R² and R³ is a group other than a hydrogen atom), provided that, in the above definitions, compounds in the following cases (1) to (20) are excluded:

(1) the case where the partial structure ZR¹—CR² is C═C; R³ is a hydrogen atom; X¹ is —CH₂CH₂—; A¹ is a p-chlorophenyl group; A² is a p-bromophenyl group; and A³ is a phenyl group, p-tolyl group or p-methoxyphenyl group, (2) the case where the partial structure ZR¹—CR² is C═C; R³ is a hydrogen atom; X² is —CH₂CH₂CH₂—; A² is a [4-(m-chlorophenyl)]piperazinyl group; and each of A¹ and A³ is a phenyl group, (3) the case where the partial structure ZR¹—CR² is C═C; R³ is a hydrogen atom; each of X¹, X² and X³ is a single bond; and each of A¹, A² and A³ is a phenyl group, (4) the case where the partial structure ZR¹—CR² is C═C; R³ is a hydrogen atom; each of X¹, X² and X³ is a single bond; each of A¹ and A² is a phenyl group; and A³ is a p-tolyl group or p-methoxyphenyl group, (5) the case where the partial structure ZR¹—CR² is C═C; R³is a hydrogen atom; each of X¹, X² and X³ is a single bond; each of A² and A³ is a phenyl group; and A¹ is a p-methoxyphenyl group, N-piperazinyl group, N-piperidinyl group or N-morpholinyl group, (6) the case where the partial structure ZR¹—CR² is C═C; R³is a hydrogen atom; each of X¹, X² and X³ is a single bond; A¹ is a 2,4,6-trimethylphenyl group; A is a phenyl group; and A³ is a 3,4-dichlorophenyl group, (7) the case where Z is C, each of R¹, R² and R³ is a hydrogen atom; each of X¹, X² and X³ is a single bond; and each of A¹, A² and A³ is a phenyl group, (8) the case where Z is C; each of R¹, R² and R³ is a hydrogen atom; each of X¹, X² and X³ is a single bond; each of A¹ and A² is a phenyl group; and A³ is a p-tolyl group, p-chlorophenyl group, p-methoxyphenyl group, 3-methoxy-4-iodophenyl group, 3-chloro-4-methoxyphenyl group, 9-anthracenyl group, 3-bromo-4-methoxyphenyl group or 4-methyl-3-iodophenyl group, (9) the case where Z is C; each of R¹, R² and R³is a hydrogen atom; each of X¹, X² and X³ is a single bond; A¹ is a 3,5-dimethyl-1H-pyrazol-1-yl group; A² is a phenyl group; and A³ is a phenyl group, p-bromophenyl group, p- chlorophenyl group, p-methoxyphenyl group, p-tolyl group, 3,4-dichlorophenyl group, 2,4-dimethylphenyl group or 3-methyl-4-chlorophenyl group, (10) the case where Z is C; each of R¹, R² and R³ is a hydrogen atom; each of X¹, X² and X³ is a single bond; A¹ is a 2,4-dimethylphenyl group; A² is a phenyl group; and A³is a phenyl group, p-tolyl group, 3,4-dichlorophenyl group, 2,4-dimethylphenyl group or 4-methyl-3-bromophenyl group, (11) the case where Z is C; each of R¹, R² and R² is a hydrogen atom; each of X¹, X² and X³ is a single bond; A¹ is a 2,4,6-trimethylphenyl group; A² is a phenyl group; and A³ is a phenyl group or 3,4-dichlorophenyl group, (12) the case where Z is C; each of R¹, R² and R³ is a hydrogen atom; each of X¹, X² and X³ is a single bond; A¹ is a 2,4,6-trimethylphenyl group; A³ is a 3,4-dinitrophenyl group; and A² is a 4-nitrophenyl group or 2,4-dinitrophenyl group, (13) the case where Z is C; each of R¹, R² and R³ is a hydrogen atom; each of X¹, X² and X³ is a single bond; A¹ is a 2,5-dimethylphenyl group; A² is a phenyl group; and A³ is a p-diphenyl group, 3,4-dichlorophenyl group or 3-methyl-4-chlorophenyl group, (14) the case where Z is C; each of R¹, R² and R³ is a hydrogen atom; each of X¹, X² and X³ is a single bond; A² is a phenyl group; A³ is a p-bromophenyl group; and A¹ is a p-tolyl group, p-ethylphenyl group or p- isopropylphenyl group, (15) the case where Z is C; each of R¹, R² and R³ is a hydrogen atom; each of X¹, X² and X³ is a single bond; A² is a phenyl group; and each of A¹ and A³ is a p-methoxyphenyl group or 3,4-dimethylphenyl group, (16) the case where Z is C; each of R¹, R² and R³ is a hydrogen atom; each of X¹, X² and X³ is a single bond; A¹ is a p-tolyl group; A² is a phenyl group; and A³ is a p-chlorophenyl group, (17) the case where Z is C; each of R, R² and R³ is a hydrogen atom; each of X¹, X² and X³ is a single bond; each of A¹ and A³ is a phenyl group; and A² is a 1-methylpiperidin-4-yl group, (18) the case where Z is C; each of R¹, R² and R³ is a hydrogen atom; each of X¹, X² and X³ is a single bond; A¹ is a 2,4,6(1H,3H,5H)-pyrimidinetrion-5-yl group; A is a phenyl group; and A³ is a 3-methyl-4-chlorophenyl group, (19) the case where Z is C; each of R¹, R² and R³ is a hydrogen atom; each of X¹, X² and X³ is a single bond; each of A¹ and A³ is a 2,4-dimethylphenyl group; and A² is a 2,4-dinitrophenyl group, and (20) the case where Z is N; X¹ is —NHCO; each of R² and R³ is a hydrogen atom; each of X² and X³ is a single bond; and each of A¹, A² and A³ is a phenyl group.

That is, the present invention relates to (1) the compound represented by the above formula (I), a salt thereof or a hydrate of them; (2) the compound according to the above (1), a salt thereof or a hydrate of them, wherein A¹, A² and/or A³ are independent of each other and each represents a C₃₋₈ cycloalkyl group, a C₃₋₈ cycloalkenyl group or a 5- to 14-membered non-aromatic heterocyclic group, each of which may be substituted; (3) the compound according to the above (1), a salt thereof or a hydrate of them, wherein A¹, A² and A³ are independent of each other and each represents a C₆₋₁₄ aromatic hydrocarbon cyclic group or 5- to 14-membered aromatic heterocyclic group, each of which may be substituted; (4) the compound according to the above (1), a salt thereof or a hydrate of them, wherein A¹, A² and A³ are independent of each other and each represents a phenyl group, pyrrolyl group, pyridyl group, pyridazinyl group, pyrimidinyl group, pyrazinyl group, thienyl group, thiazolyl group, furyl group, naphthyl group, quinolyl group, isoquinolyl group, indolyl group, benzimidazolyl group, benzothiazolyl group, benzoxazolyl group, imidazopyridyl group, carbazolyl group, cyclopentyl group, cyclohexyl group, cyclohexenyl group, dioxinyl group, adamantyl group, pyrrolidinyl group, piperidinyl group, piperazinyl group or morpholinyl group, each of which may be substituted; (5) the compound according to the above (1), a salt thereof or a hydrate of them, wherein A¹, A² and A³ are independent of each other and each represents a group represented by the formula: 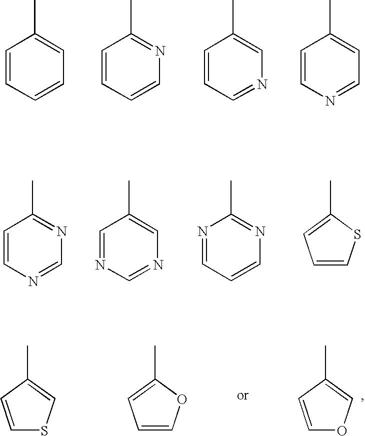
each of which may be substituted; (6) the compound according to the above (1), a salt thereof or a hydrate of them, wherein X¹, X² and X³ are independent of each other and each represents (a) a single bond, (b) a C₁₋₆ alkylene group, a C₂₋₆ alkenylene group or a C₂₋₆ alkynylene group, each of which may be substituted with one or more groups selected from the substituent group a below, (c) —NH—, (d) —O—, (e) —N(R⁴)CO—, (f) —CON(R⁵)—, (g) —N(R⁶)CH₂—, (h) —CH₂N(R⁷)—, (i) —CH₂CO—, (j) —COCH₂—, (k) —N(R⁸)SO₀₋₂—, (l) —S₀₋₂N(R⁹)—, (m) —CH₂SO₀₋₂—, (n) —SO₀₋₂CH₂—, (o) —CH₂O—, (p) —OCH₂—, (q) —N(R¹⁰)CON(R¹¹)—, (r) —N(R¹²)CS—N(R¹³)— or (s) —SO₀₋₂— (wherein R⁴, R⁵, R⁶, R⁷, R⁸, R⁹, R¹⁰, R¹¹, R¹² and R¹³ have the same meanings as defined in the above-mentioned (1), respectively); and A¹, A² and A³ are independent of each other and each represents a C₃₋₈ cycloalkyl group, a C₃₋₈ cycloalkenyl group, a 5- to 14-membered non-aromatic heterocyclic group, a C₆₋₁₄ aromatic hydrocarbon cyclic group or a 5- to 14-membered aromatic heterocyclic group, each of which may be substituted with one or more groups selected from the substituent group b below:
<substituent group a>the group consisting of a hydroxyl group, a halogen atom and a cyano group;
<substituent group b>the group consisting of (a) a hydroxyl group, (b) a halogen atom, (c) a nitrile group, (d) a nitro group, (e) a C₁₋₆ alkyl group, C₂₋₆ alkenyl group or C₂₋₆ alkynyl group, each of which may be substituted with at least one group selected from the group consisting of a hydroxyl group, nitrile group, halogen atom, C₁₋₆ alkylamino group, di(C₁₋₆ alkyl)amino group, C₂₋₆ alkenylamino group, di(C₂₋₆ alkenyl)amino group, C₂₋₆ alkynylamino group, di(C₂₋₆ alkynyl)amino group, N—C₂₋₆ alkyl-N—C₂₋₆ alkenylamino group, N—C₁₋₆ alkyl-N—C₂₋₆ alkynylamino group, N—C₂₋₆ alkenyl-N—C₂₋₆ alkynylamino group, aralkyloxy group, TBDMS oxy group, C₁₋₆ alkylsulfonylamino group, C₁₋₆ alkylcarbonyloxy group, C₂₋₆ alkenylcarbonyloxy group, C₂₋₆ alkynylcarbonyloxy group, N—C₁₋₆ alkylcarbamoyl group, N—C₂₋₆ alkenylcarbamoyl group and N—C₂₋₆ alkynylcarbamoyl group, (f) a C₁₋₆ alkoxy group, C_{2- 6} alkenyloxy group or C₂₋₆ alkynyloxy group, each of which may be substituted with at least one group selected from the group consisting of a C₁₋₆ alkylamino group, aralkyloxy group and hydroxyl group, (g) a C₁₋₆ alkylthio group, C₂₋₆ alkenylthio group or C₂₋₆ alkynylthio group, each of which may be substituted with at least one group selected from the group consisting of a hydroxyl group, nitrile group, halogen atom, C₁₋₆ alkylamino group, aralkyloxy group, TBDMS oxy group, C₁₋₆ alkylsulfonylamino group, C₁₋₆ alkylcarbonyloxy group and C₁₋₆ alkylcarbamoyl group, (h) a carbonyl group substituted with a group selected from the group consisting of a C₁₋₆ alkoxy group, amino group, C₁₋₆ alkylamino group, di(C₁₋₆ alkyl)amino group, C₂₋₆ alkenylamino group, di(C₂₋₆ alkenyl)amino group, C₂₋₆ alkynylamino group, di(C₂₋₆ alkynyl)amino group, N—C₁₋₆ alkyl-N—C₂₋₆ alkenylamino group, N—C₁₋₆ alkyl-N—C₂₋₆ alkynylamino group and N—C₂₋₆ alkenyl-N—C₂₋₆ alkynylamino group, (i) an amino group which may be substituted with one or two groups selected from the group consisting of a C₁₋₆ alkyl group, C₂₋₆ alkenyl group, C₂₋₆ alkynyl group, C₁₋₆ alkylsulfonyl group, C₂₋₆ alkenylsulfonyl group, C₂₋₆ alkynylsulfonyl group, C₁₋₆ alkylcarbonyl group, C₂₋₆ alkenylcarbonyl group and C₂₋₆ alkynylcarbonyl group, (j) a C₁₋₆ alkylsulfonyl group, (k) a C₂₋₆ alkenylsulfonyl group, (l) a C₂₋₆ alkynylsulfonyl group, (m) a C₁₋₆ alkylsulfinyl group, (n) a C₂₋₆ alkenylsulfinyl group, (o) a C₂₋₆ alkynylsulfinyl group, (p) a formyl group, (q) a C₃₋₈ cycloalkyl group or C₃₋₈ cycloalkenyl group, each of which may be substituted with at least one group selected from the group consisting of a hydroxyl group, halogen atom, nitrile group, C₁₋₆ alkyl group, C₁₋₆ alkoxy group, C₁₋₆ alkoxy-C₁₋₆ alkyl group and aralkyl group, (r) a 5- to 14-membered non-aromatic heterocyclic group which may be substituted with at least one group selected from the group consisting of a hydroxyl group, halogen atom, nitrile group, C₁₋₆ alkyl group, C₁₋₆ alkoxy group, C₁₋₆ alkoxy-C₁₋₆ alkyl group and aralkyl group, (s) a C₁₋₆ aromatic hydrocarbon cyclic group which may be substituted with at least one group selected from the group consisting of a hydroxyl group, halogen atom, nitrile group, C₁₋₆ alkyl group, C₁₋₆ alkoxy group, C₁₋₆ alkoxy-C₁₋₆ alkyl group and aralkyl group, and (t) a 5- to 14-membered aromatic heterocyclic group which may be substituted with at least one group selected from the group consisting of a hydroxyl group, halogen atom, nitrile group, C₁₋₆ alkyl group, C₁₋₆ alkoxy group, C₁₋₆ alkoxy-C₁₋₆ alkyl group and aralkyl group, and (u) thiol group; (7) the compound according to the above (1), a salt thereof or a hydrate of them, wherein substituent groups on A¹, A² and/or A³ are independent of each other and each represents a hydroxyl group, a halogen atom, a nitrile group or a nitro group; (8) the compound according to the above (1), a salt thereof or a hydrate of them, wherein Q is O; (9) the compound according to the above (1), a salt thereof or a hydrate of them, wherein X¹, X² and X³ are independent of each other and each represents a single bond, —CH₂—, —CH(OH)—, —CH₂CH₂—, —CH═CH— or —C≡C—; (10) the compound according to the above (1), a salt thereof or a hydrate of them, wherein X¹, X² and X³ each represents a single bond; (11) the compound according to the above (1), a salt thereof or a hydrate of them, wherein R¹, R² and/or R³ represent an optionally substituted C₁₋₆ alkyl group; (12) the compound according to the above (1), a salt thereof or a hydrate of them, wherein R¹, R² and/or R³ each represents a hydrogen atom; (13) the compound according to the above (1), a salt thereof or a hydrate of them, wherein R¹ and R² are bound to each other such that the partial structure ZR¹—CR² forms a carbon-carbon double bond represented by the formula C═C; (14) the compound according to the above (1), a salt thereof or a hydrate of them, wherein R³ is bound to an atom in A¹ to form a ring with the atom and X¹; (15) the compound according to the above (1), a salt thereof or a hydrate of them, wherein R³ is bound to an atom in A³ to form a ring with the atom and X³; (16) the compound according to the above (14) or (15), a salt thereof or a hydrate of them, wherein the ring formed by R³ is (a) an optionally substituted C₅₋₈ hydrocarbon ring or (b) a 5- to 8-membered heterocyclic ring which contains an oxygen atom and is optionally substituted; (17) the compound according to any one of (14) to (16), a salt thereof or a hydrate of them, wherein X³ is a single bond; (18) the compound according to the above (1), a salt thereof or a hydrate of them, wherein the binding positions of substituent groups on A¹, A² and/or A³ are α-positions of the carbon atoms on A¹, A² and/or A³, each of which are bound to X¹, X² and X³, respectively; (19) a compound represented by the following formula, a salt thereof or a hydrate of them: 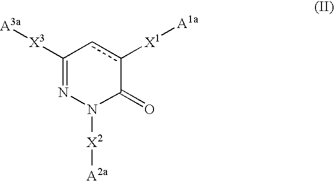
wherein A^{1a}, A^{2a} and A^{3a} are independent of each other and each represents a C₆₋₁₄ aromatic hydrocarbon cyclic group or a 5- to 14-membered aromatic heterocyclic group, each of which may be substituted; X¹, X² and X³ have the same meanings as defined in the above-mentioned (1), respectively; and the partial structure: [symbol]
represents a single or double bond, provided that, in the above-mentioned definitions, compounds in the following cases (1) and (2) are excluded:
(1) the case where the partial structure: [symbol]
is a carbon-carbon double bond; R³ is a hydrogen atom; and the following cases (1a) to (1f) stand:
(1a) the case where X¹ is —CH₂CH₂—; A¹ is a p-chlorophenyl group; A² is a p-bromophenyl group; and A³is a phenyl group, p-tolyl group or p-methoxyphenyl group,
(1b) the case where X² is —CH₂CH₂CH₂—; A² is a [4-(m-chlorophenyl)]piperazinyl group; and each of A¹ and A² is a phenyl group, (1c) the case where each of X¹, X² and X³ is a single bond; and each of A¹, A² and A³ is a phenyl group, (Id) the case where each of X¹, X² and X³ is a single bond; each of A¹ and A² is a phenyl group; and A³ is a p-tolyl group or p-methoxyphenyl group, (1e) the case where each of X¹, X² and X³ is a single bond; each of A² and A³is a phenyl group; and A¹ is a p-methoxyphenyl group, N-piperazinyl group, N-piperidinyl group or N-morpholinyl group, and (1f) the case where each of X¹, X² and X³ is a single bond; A¹ is a 2,4,6-trimethylphenyl group; A² is a phenyl group; and A³is a 3,4-dichlorophenyl group, and
(2) the case where the partial structure: is a single bond; each of X¹, X² and X³ is a single bond; and the following cases (2a) to (2m) stand:
(2a) the case where each of A¹, A² and A³is a phenyl group, (2b) the case where each of A¹ and A² is a phenyl group; and A³ is a p-tolyl group, p-chlorophenyl group, p-methoxyphenyl group, 3-methoxy-4-iodophenyl group, 3-chloro-4-methoxyphenyl group, 9-anthracenyl group, 3-bromo-4-methoxyphenyl group or 4-methyl-3-iodophenyl group, (2c) the case where A¹ is a 3,5-dimethyl-1H-pyrazol-1-yl group; A² is a phenyl group; and A³ is a phenyl group, p-bromophenyl group, p-chlorophenyl group, p-methoxyphenyl group, p-tolyl group, 3,4-dichlorophenyl group, 2,4-dimethylphenyl group or 3-methyl-4-chlorophenyl group, (2d) the case where A¹ is a 2,4-dimethylphenyl group; A² is a phenyl group; and A³is a phenyl group, p-tolyl group, 3,4-dichlorophenyl group, 2,4-dimethylphenyl group or 4-methyl-3-bromophenyl group, (2e) the case where A¹ is a 2,4,6-trimethylphenyl group; A² is a phenyl group; and A³is a phenyl group or 3,4-dichlorophenyl group, (2f) the case where A¹ is a 2,4,6-trimethylphenyl group; A³ is a 3,4-dichlorophenyl group; and A is a 4-nitrophenyl group or 2,4-dinitrophenyl group, (2g) the case where A¹ is a 2,5-dimethylphenyl group; A² is a phenyl group; and A³ is a p-diphenyl group, 3,4-dichlorophenyl group or 3-methyl-4-chlorophenyl group, (2h) the case where A² is a phenyl group; A³is a p-bromophenyl group; and A¹ is a p-tolyl group, p-ethylphenyl group or p-isopropylphenyl group, (2i) the case where A² is a phenyl group; and A¹ and A³ are independent of each other and each represents a p-methoxyphenyl group or 3,4-dimethylphenyl group, (2j) the case where A¹ is a p-tolyl group; A² is a phenyl group; and A³is a p-chlorophenyl group, (2k) the case where each of A¹ and A³ is a phenyl group; and A² is a 1-methylpiperidin-4-yl group, (21) the case where A¹ is a 2,4,6(1H,3H,5H)-pyrimidinetrion-5-yl group; A² is a phenyl group; and A³ is a 3-methyl-4-chlorophenyl group, and (2m) the case where each of A¹ and A³ is a 2,4-dimethylphenyl group; and A² is a 2,4-dinitrophenyl group; (20) the compound according to the above-mentioned (19), a salt thereof or a hydrate of them, wherein A^{1a} A^{2a} and A^{3a} independently represents a phenyl group, pyrrolyl group, pyridyl group, pyridazinyl group, pyrimidinyl group, pyrazinyl group, thienyl group, thiazolyl group, furyl group, naphthyl group, quinolyl group, isoquinolyl group, indolyl group, benzimidazolyl group, benzothiazolyl group, benzoxazolyl group, imidazopyridyl group, carbazolyl group, cyclopentyl group, cyclohexyl group, cyclohexenyl group, dioxynyl group, adamantyl group, pyrrolidinyl group, piperidinyl group, piperazinyl group or morpholinyl group, each of which may be substituted; (21) the compound according to the above-mentioned (19), a salt thereof or a hydrate of them, wherein each of X¹, X² and X³ is a single bond; (22) the compound according to the above (1), a salt thereof or a hydrate of them, which is represented by the formula: 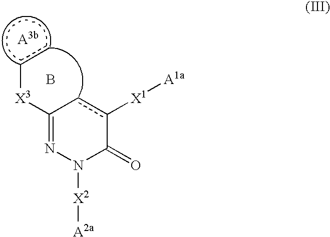
wherein, A^{1a}, A^{2a} and the partial structure: [symbol]
have the same meanings as defined in the above (19), respectively; X¹, X² and X³ have the same meanings as defined in the above (1), respectively; the ring A^{3b} represents a C₆₋₈ aromatic hydrocarbon ring or a 5- to 8-membered aromatic heterocyclic ring, each of which may be substituted; and the ring B represents (a) an optionally substituted C₅₋₉ cycloalkane or C₅₋₉ cycloalkene or (b) a 5- to 9-membered non-aromatic heterocyclic ring which contains a hetero atom selected from the group consisting of N, O and S, and may be substituted; (23) the compound according to the above (22), a salt thereof or a hydrate of them, wherein A^{1a}, A^{2a} and A^{3b} are independent of each other and each represents a phenyl group, pyrrolyl group, pyridyl group, pyridazinyl group, pyrimidinyl group, pyrazinyl group, thienyl group, thiazolyl group, furyl group, naphthyl group, quinolyl group, isoquinolyl group, indolyl group, benzimidazolyl group, benzothiazolyl group, benzoxazolyl group, imidazopyridyl group, carbazolyl group, cyclopentyl group, cyclohexyl group, cyclohexenyl group, dioxinyl group, adamantyl group, pyrrolidinyl group, piperidinyl group, piperazinyl group or morpholinyl group, each of which may be substituted; (24) the compound according to the above (1), a salt thereof or a hydrate of them, which represented by the formula: 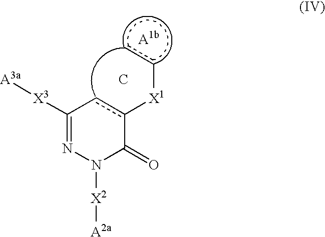
wherein A^{2a}, A^{3a} and the partial structure: [symbol]
have the same meanings as defined in the above (19), respectively; X¹, X² and X³ have the same meanings as defined in the above (1), respectively; the ring A^{1b} represents a C₆₋₈ aromatic hydrocarbon ring or a 5- to 8-membered aromatic heterocyclic ring, each of which may be substituted; and the ring C represents (a) an optionally substituted C₅₋₉ cycloalkane or C₅₋₉ cycloalkene or (b) a 5- to 9-membered non-aromatic heterocyclic ring which contains a hetero atom selected from the group consisting of N, O and S, and may be substituted; (25) the compound according to the above-mentioned (24), a salt thereof or a hydrate of them, wherein A^{1b}, A^{2a} and A^{3a} independently represents a phenyl group, pyrrolyl group, pyridyl group, pyridazinyl group, pyrimidinyl group, pyrazinyl group, thienyl group, thiazolyl group, furyl group, naphthyl group, quinolyl group, isoquinolyl group, indolyl group, benzimidazolyl group, benzothiazolyl group, benzoxazolyl group, imidazopyridyl group, carbazolyl group, cyclopentyl group, cyclohexyl group, cyclohexenyl group, dioxynyl group, adamantyl group, pyrrolidinyl group, piperidinyl group, piperazinyl group or morpholinyl group, each of which may be substituted; (26) the compound according to the above (22), a salt thereof or a hydrate of them, which is represented by the formula: 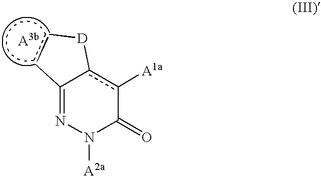
wherein A1a, A^{2a}, A^{3b} and the partial structure: [symbol]
have the same meanings as defined in the above (22); and D represents a group represented by —CH₂—, —(CH₂)₂—, —C═C—, —C≡C—, —O—, —OCH₂—, —CH₂O—, —SO_{0-2—}, —SCH₂—, —CH₂S—, —SOCH₂—, —CH₂SO—, —SO₂CH₂—, —CH₂SO₂—, —NR¹⁴—, —NR¹⁴CH₂— or —CH₂NR¹⁴— (wherein, R¹⁴ represents a hydrogen atom, a C₁₋₆ alkyl group, an optionally substituted C₃₋₈ cycloalkyl group, an optionally substituted 5- to 14-membered non-aromatic heterocyclic group, an optionally substituted C₆₋₁₄ aromatic hydrocarbon cyclic group or an optionally substituted 5- to 14-membered aromatic heterocyclic group), and the substitutable positions in D may be substituted; (27) the compound according to the above (24), a salt thereof or a hydrate of them, which is represented by the formula: 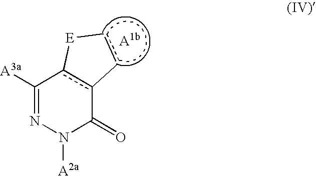
wherein A^{1b}, A^{2a}, A^{3a} and the partial structure: [symbol]
have the same meanings as defined in the above (24), respectively; and E represents —CH₂—, —CH₂)₂—, —C═C—, —C≡C—, —O—, —OCH₂—, —CH₂O—, —SO₀₋₂—, —SCH₂—, —CH₂S—, —SOCH₂—, —CH₂SO—, —SO₂CH₂—, —CH₂SO₂—, —NR¹⁴—, —NR¹⁴CH₂— or —CH₂NR¹⁴— (wherein, R¹⁴ has the same meaning as defined in the above (26)), and the substitutable positions in E may be substituted; (28) the compound according to the above (1), a salt thereof or a hydrate of them, which is represented by the formula: 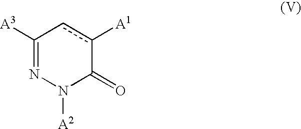
wherein A¹, A², A³ and the partial structure: [symbol]
have the same meanings as defined above, respectively; (29) the compound according to the above (1), a salt thereof or a hydrate of them, which is represented by the formula: 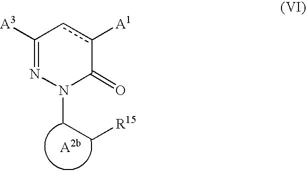
wherein A¹, A³ and the partial structure: [symbol]
have the same meanings as defined above, respectively; the ring A^{2b} represents a C₆₋₁₄ aromatic hydrocarbon ring or a 5- to 14-membered aromatic heterocyclic ring, each of which may be further substituted; and R¹⁵ represents a hydroxyl group, a halogen atom, a nitrile group, a C₁₋₆ alkyl group, a C₁₋₆ alkoxy group, a nitro group, an amino group, a C₁₋₆ alkylamino group, a formyl group, a C₁₋₆ alkylcarbonyl group or a trifluoromethyl group; (30) the compound according to the above (29), a salt thereof or a hydrate of them, wherein A¹, A^{2b} and A³ are independent of each other and each represents a phenyl group, pyrrolyl group, pyridyl group, pyridazinyl group, pyrimidinyl group, pyrazinyl group, thienyl group, thiazolyl group, furyl group, naphthyl group, quinolyl group, isoquinolyl group, indolyl group, benzimidazolyl group, benzothiazolyl group, benzoxazolyl group, imidazopyridyl group, carbazolyl group, cyclopentyl group, cyclohexyl group, cyclohexenyl group, dioxinyl group, adamantyl group, pyrrolidinyl group, piperidinyl group, piperazinyl group or morpholinyl group, each of which may be substituted; (31) the compound according to the above (1), a salt thereof or a hydrate of them, which is represented by the formula: 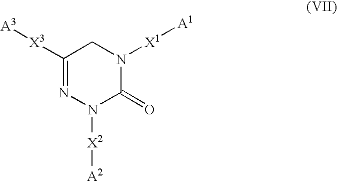
wherein A¹, A², A³, X¹, X² and X³ have the same meanings as defined above, respectively, provided that compounds in the following cases (a) to (d):
(a) the case where X¹ is —NHCO; each of X² and X³ is a single bond; and each of A¹, A² and A³ is a phenyl group, (b) the case where each of X¹, X² and X³ is a single bond; and each of A¹, A² and A³ is a phenyl group, (c) the case where each of X¹, X² and X³ is a single bond; A¹ is an o,p-dimethylphenyl group; A² is an o-methylphenyl group; and A³ is a phenyl group, and (d) the case where each of X¹, X² and X³ is a single bond; A¹ is an o-methylphenyl group; A² is a p-methoxyphenyl group; and A³is a phenyl group are excluded; (32) the compound according to the above (1), a salt thereof or a hydrate of them, which is represented by the formula: 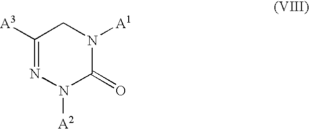
wherein A¹, A² and A³ have the same meanings as defined in the above-mentioned (1), respectively, provided that compounds in the following cases (a) to (c): (a) the case where each of A¹, A² and A³ is a phenyl group, (b) the case where A¹ is an o,p-dimethylphenyl group; A² is an o-methylphenyl group; and A³ is a phenyl group, and (c) the case where A¹ is an o-methylphenyl group; A²is a p-methoxyphenyl group; and A³ is a phenyl group are excluded; (33) the compound according to the above (32), a salt thereof or a hydrate of them, wherein A¹, A² and A³ are independent of each other and each represents a C₆₋₁₄ aromatic hydrocarbon cyclic group or a 5- to 14-membered aromatic heterocyclic group, each of which may be substituted; (34) the compound according to the above (32), a salt thereof or a hydrate of them, wherein A¹, A² and A³ are independent of each other and each represents a phenyl group, pyrrolyl group, pyridyl group, pyridazinyl group, pyrimidinyl group, pyrazinyl group, thienyl group, thiazolyl group, furyl group, naphthyl group, quinolyl group, isoquinolyl group, indolyl group, benzimidazolyl group, benzothiazolyl group, benzoxazolyl group, imidazopyridyl group, carbazolyl group, cyclopentyl group, cyclohexyl group, cyclohexenyl group, dioxinyl group, adamantyl group, pyrrolidinyl group, piperidinyl group, piperazinyl group or morpholinyl group, each of which may be substituted; (35) the compound according to the above (32), a salt thereof or a hydrate of them, wherein A¹, A² and A³ are independent of each other and each represents a group represented by the formula: 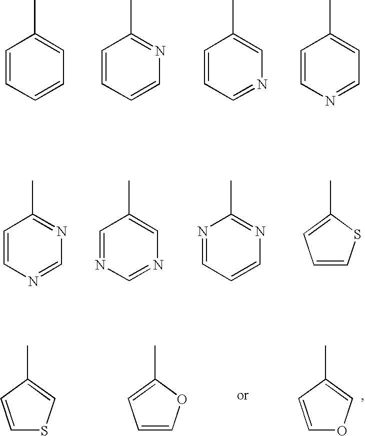
each of which may be substituted; (36) the compound according to the above (32), a salt thereof or a hydrate of them, wherein each of A¹, A² and A³ may be substituted with at least one group selected independently from the group consisting of a halogen atom, cyano group, hydroxyl group, amino group, formyl group and nitro group; (37) the compound according to the above- mentioned (32), a salt thereof or a hydrate of them, wherein the binding positions of substituent groups on A¹, A² and/or A³ are α-positions of the carbon atoms on A¹, A² and/or A³, each of which are bound directly to the triazinone ring; (38) the compound according to the above (1), a salt thereof or a hydrate of them, which is represented by the following formula: 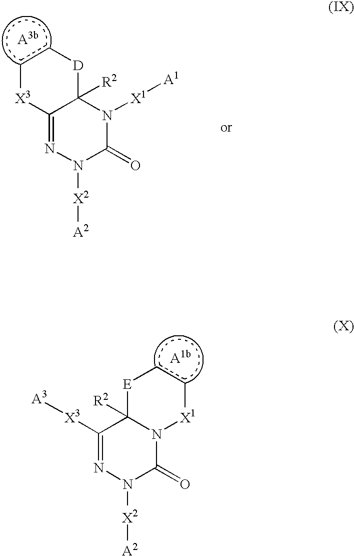
wherein, A¹, A², A³, A^{1b}, A^{3b}, X¹,X², X³, D, E and R² have the same meanings as defined above, respectively; (39) the compound according to the above (1), a salt thereof or a hydarate of them, which is any one of compounds selected from 2-(2-bromophenyl)-4-(3-methoxyphenyl)-6-(2-pyridyl)-4,5-dihydro-3(2H)-pyridazinone,2-(2-bromophenyl-4-(3-hydroxyphenyl)-6-(2-pyridyl)-4,5-dihydro-3(2H)-pyridazinone, 2-(2-bromophenyl)-4-[3-(2-hydroxyethoxy)phenyl]-6-(2-pyridyl)-3(2H)-pyridazinone, 2-(2-cyanophenyl)-4-[3-(2-hydroxyethoxy)phenyl]-6-(2-pyridyl)-3(2H)-pyridazinone, 2-(2-bromophenyl)-6-(2-methoxyphenyl)-4-(2-pyridyl)-3(2H)-pyridazinone, 2-(2-cyanophenyl)-4-phenyl-2,3,4,4a-tetrahydro-5H-(1)benzopyrano[4,3-c]pyridazin-3-one, 2-(2-cyanophenyl)-4-phenyl-2,3-dihydro-5H-(1)benzopyrano[4,3-c]pyridazin-3-one, 2-(2-iodophenyl)-4-(3-pyridyl)-2,3,4,4a-tetrahydro-5H-(1)benzopyrano[4,3-c]pyridazin-3-one, 2-(2-cyanophenyl)-4-(3-pyridyl)-2,3-dihydro-5H-(1)benzopyrano[4,3-c]pyridazin-3-one, 4-(4-methoxybenzyl)-6-phenyl-2-(2-tolyl)-3(2H)-pyridazinone, 2,6-diphenyl-4-(α-hydroxy-2-picolyl)-4,5-dihydro-3(2H)-pyridazinone, 2-(2-cyanophenyl)-4-(4-morpholinoethylaminocarbonyl)-6-phenyl-3(2H)-pyridazinone, 2-(2-cyanophenyl)-6-(2-pyridyl)-4,5-dihydro-2H-pyridazino[4,5-b]benzofuran-3-one, 2-(2-bromophenyl)-4-(2-methoxyphenyl)-6-(2-pyridyl)-4,5-dihydro-3(2H)-pyridazinone, 2-(2-bromophenyl)-4-(4-methoxyphenyl)-6-(2-pyridyl)-4,5-dihydro-3(2H)-pyridazinone, 2-(2-bromophenyl)-4-(3-bromo-6-methoxyphenyl)-6-(2-pyridyl)-4,5-dihydro-3(2H)-pyridazinone, 2-(2-iodophenyl)-4-(2-methoxyphenyl)-6-(2-pyridyl)-4,5-dihydro-3(2H)-pyridazinone, 4-(2-methoxyphenyl)-2-phenyl-6-(2-pyridyl)-4,5-dihydro-3(2H)-pyridazinone, 2-(2-bromophenyl)-4-phenyl-6-(2-pyridyl)-4,5-dihydro-3(2H)-pyridazinone, 2-(2-bromophenyl)-4-phenyl-6-(3-pyridyl)-4,5-dihydro-3(2H)-pyridazinone, 4,6-diphenyl-2-(2-pyridyl)-4,5-dihydro-3(2H)-pyridazinone, 4-(2-methoxyphenyl)-2-(2-pyridyl)-6-(2-pyridyl)-4,5-dihydro-3(2H)-pyridazinone, 4-(2-cyanophenyl)-2-phenyl-6-(2-pyridyl)-3(2H)-pyridazinone, 2-(2-bromophenyl)-4-(2-methoxyphenyl)-6-(3-pyridyl)-4,5-dihydro-3(2H)-pyridazinone, 4-(2-bromophenyl)-2-phenyl-6-(2-pyridyl)-4,5-dihydro-3(2H)-pyridazinone, 2-(2-methoxyphenyl)-4-phenyl-6-(2-pyridyl)-4,5-dihydro-3(2H)-pyridazinone, 4-phenyl-2-(2-nitrophenyl)-6-(2-pyridyl)-4,5-dihydro-3(2H)-pyridazinone, 2-(2-fluorophenyl)-4-phenyl-6-(2-pyridyl)-4,5-dihydro-3(2H)-pyridazinone, 2-(2-bromophenyl)-4-(2-hydroxyphenyl)-6-(2-pyridyl)-4,5-dihydro-3(2H)-pyridazinone, 2-(2-bromophenyl)-4-(4-hydroxyphenyl)-6-(2-pyridyl)-4,5-dihydro-3(2H)- pyridazinone, 2-(2-bromophenyl)-6-(2-hydroxyphenyl)-4-(2-pyridyl)-3(2H)-pyridazinone, 4-(2-hydroxyphenyl)-2-phenyl-6-(2-pyridyl)-3(2H)-pyridazinone, 4-(2-hydroxyphenyl)-2-phenyl-6-(2-pyridyl)-4,5-dihydro-3(2H)-pyridazinone, 2-(2-bromophenyl)-4-(2-hydroxyphenyl)-6-(3-pyridyl)-4,5-dihydro-3(2H-pyridazinone, 2-(2-bromophenyl)-4-(2-dimethylaminoethoxyphenyl)-6-(2-pyridyl)-3(2H)-pyridazinone, 2-(2-bromophenyl)-6-(2-dimethylaminoethoxyphenyl)-4-(2-pyridyl)-3(2H)-pyridazinone, 2-(2-bromophenyl)-4-[3-(2-picolyloxyphenyl)]-6-(2-pyridyl)-3(2H)-pyridazinone, 2-phenyl-6-(2-pyridyl)-4-(2-trifluoromethylsulfonyloxyphenyl)-4,5-dihydro-3(2H)-pyridazinone, 2-(2-cyanophenyl)-4-(2-methoxyphenyl)-6-(2-pyridyl)-4,5-dihydro-3(2H)-pyridazinone, 2-(2-cyanophenyl)-4-(2-methoxyphenyl)-6-(2-pyridyl)-3(2H)-pyridazinone, 2-(2-cyanophenyl)-4-(2-hydroxyphenyl)-6-(2-pyridyl)-4,5-dihydro-3(2H)-pyridazinone, 2-(2-cyanophenyl)-4-(2-hydroxyphenyl)-6-(2-pyridyl)-3(2H)-pyridazinone, 2-(2-cyanophenyl)-4-phenyl-6-(2-pyridyl)-3(2H)-pyridazinone, 2-(2-cyanophenyl)-4-(3-bromo-6-methoxyphenyl)-6-(2-pyridyl)-3(2H)-pyridazinone, 2-(2-cyanophenyl)-4-(3-pyridyl)-6-(2-pyridyl)-3(2H)-pyridazinone, 2-(2-cyanophenyl)-4-(2-cyanophenyl)-6-(2-pyridyl)-3(2H)-pyridazinone, 4-(2-bromophenyl)-2-(2-cyanophenyl)-6-(2-pyridyl)-3(2H)- pyridazinone, 2-(2-cyanophenyl)-9-fluoro-4-phenyl-2,3,4,4a-tetrahydro-5H-(1)benzopyrano[4,3-c]pyridazin-3-one, 2-(2-cyanophenyl)-4-(3-pyridyl)-2,3,4,4a-tetrahydro-5H-(1)benzopyrano[4,3-c]pyridazin-3-one, 2-(2-bromophenyl)-4-(3-pyridyl)-2,3,4,4a-tetrahydro-5H-(1)benzopyrano[4,3-c]pyridazin-3-one, 2-(2-bromophenyl)-4-(2-hydroxyphenyl)-6-(2-pyridyl)-3(2H)-pyridazinone, 2-(2-bromophenyl)-4-(3-methoxyphenyl)-6-(2-pyridyl)-3(2H)-pyridazinone, 2-(2-cyanophenyl)-9-fluoro-5-hydroxy-4-phenyl-2,3-dihydro-5H-(1)benzopyrano[4,3-c]pyridazin-3-one, 2-(2-cyanophenyl)-9-fluoro-4-phenyl-2,3-dihydro-5H-(1)benzopyrano[4,3-c]pyridazin-3-one, 2-phenyl-6-(2-pyridyl)-4-(2-trifluoro-methylsulfonyloxyphenyl)-3(2H)-pyridazinone, 2-(2-bromophenyl)-4-phenyl-6-(2-pyridyl)-3(2H)-pyridazinone, 2-(2-bromophenyl)-4-(3-pyridyl)-2,3-dihydro-5H-(1)benzopyrano[4,3-c]pyridazine-3-one, 2-(2-iodophenyl)-4-(3-pyridyl)-2,3-dihydro-5H-(1)benzopyrano[4,3-c]pyridazin-3-one, 2-phenyl-4-(3-pyridyl)-6-(2-pyridyl)-3(2H)-pyridazinone, 4-(2-bromophenyl)-2-phenyl-6-(2-pyridyl)-3(2H)-pyridazinone, 2-(2-bromophenyl)-4-(3-pyridyl)-6-(2-pyridyl)-3(2H)-pyridazinone, 2-(2-chlorophenyl)-4-(4-morpholinoethylaminocarbonyl)-6-phenyl-3(2H)-pyridazinone, 2-(2-nitrophenyl)-4-(3-pyridyl)-6-(2-pyridyl)-3(2H)-pyridazinone, 2-(3-tolyl)-4-(3-pyridyl)-6-(2-pyridyl)-3(2H)-pyridazinone, 2-(4-methanesulfonylphenyl)-4-(3-pyridyl)-6-(2-pyridyl)-3(2H)-pyridazinone, 2-(4-biphenyl)-4-(3-pyridyl)-6-(2-pyridyl)-3(2H)-pyridazinone, 2-(2-naphthyl)-4-(3-pyridyl)-6-(2-pyridyl)-3(2H)-pyridazinone, 2-(3,4-methylenedioxyphenyl)-4-(3-pyridyl)-6-(2-pyridyl)-3(2H)-pyridazinone, 2-(3,4-dichlorophenyl)-4-(3-pyridyl)-6-(2-pyridyl)-3(2H)-pyridazinone, 2-(2-cyanophenyl)-4-phenyl-6-(2-pyrimidinyl)-3(2H)-pyridazinone, 2-(2-pyridyl)-4-(2-pyridyl)-6-(2-methoxyphenyl)-3(2H)-pyridazinone, 2-(3-formylphenyl)-4-(3-pyridyl)-6-(2-pyridyl)-3(2H)-pyridazinone, 2-(thiophen-3-yl)-4-(3-pyridyl)-6-(2-pyridyl)-3(2H)-pyridazinone, 2-(3-pyridyl)-4-phenyl-6-(2-pyridyl)-3(2H)-pyridazinone, 2-(3-pyridyl)-4-phenyl-6-(2-pyrimidinyl)-3 (2H)-pyridazinone, 2-(2-methoxyphenyl)-4-(3-pyridyl)-6-(2-pyridyl)-4,5-dihydro-3(2H)-pyridazinone, 4-methyl-2,4,6-triphenyl-4,5-dihydro-3(2H)-pyridazinone, 2-(2-bromophenyl)-4-methyl-4,6-diphenyl-4,5-dihydro-3(2H)-pyridazinone, 2-(3-pyridin-1-oxide)-4-phenyl-6-(2-pyridyl)-3(2H)-pyridazinone, 2-(2-cyanopyridin-5-yl)-4-phenyl-6-(2-pyridyl)-3(2H)-pyridazinone, 2-(2-cyanopyridin-3-yl)-4-phenyl-6-(2-pyridyl)-3(2H)-pyridazinone, 2-(2-cyanopyrid- 5-yl)-4-phenyl-6-(2-pyrimidinyl)-3(2H)-pyridazinone, 2-(2-cyanopyridin-3-yl)-4-phenyl-6-(2-pyrimidinyl)-3(2H)-pyridazinone, 2-(2-cyanophenyl)-4-phenyl-6-(2-pyrazinyl)-3(2H)-pyridazinone, 2-(2-cyanophenyl)-4-phenyl-6-(thiazol-2-yl)-3(2H)-pyridazinone, 2-(2-cyanophenyl)-4-methyl-4,6-diphenyl-4,5-dihydro-3(2H)-pyridazinone, 2-(2-bromophenyl)-4-(2-methoxyphenyl)-6-(2-pyridyl)-4,5-dihydro-1,2,4-triazin-3(2H)-one, 2-(2-bromophenyl)-4-(2-hydroxyphenyl)-6-(2-pyridyl)-4,5-dihydro-1,2,4-triazin-3(2H)-one, 2-(2-cyanophenyl)-4-(2-methoxyphenyl)-6-(2-pyridyl)-4,5-dihydro-1,2,4-triazin-3(2H)-one, 2-(2-bromophenyl)-4-phenyl-6-(2-pyridyl)-4,5-dihydro-1,2,4-triazin-3(2H)-one, 2-bromophenyl)-6-(2-methoxyphenyl)-4-phenyl-4,5-dihydro-1,2,4-triazin-3 (2H)-one, 2-(2-bromophenyl)-6-(2-hydroxyphenyl)-4-phenyl-4,5-dihydro-1,2,4-triazin-3(2H)-one, 2-(2-bromophenyl)-6-(2-dimethylaminoethoxyphenyl)-4-phenyl-4,5-dihydro-1,2,4-triazin-3(2H)-one, 2-(2-bromophenyl)-6-(2-methoxyphenyl)-4-(2-pyridyl)-4,5-dihydro-1,2,4-triazin-3(2H)-one, 2-(2-cyanophenyl)-6-(2-hydroxyphenyl)-4-phenyl-4,5-dihydro-1,2,4-triazin-3(2H)-one, 2-(2-bromophenyl)-4-(2,5-dihydroxyphenyl)-6-(2-hydroxyphenyl)-4,5-dihydro-1,2,4-triazin-3(2H)-one, 4-(2,5-dihydroxyphenyl)-6-(2-hydroxyphenyl)-2-phenyl-4,5-dihydro-1,2,4-triazin-3(2H)-one, 2-(2-cyanophenyl)-4-(2-hydroxyphenyl)-6-(2-pyridyl)-4,5-dihydro-1,2,4-triazin-3(2H)-one, 2-(2-cyanophenyl)-6-(2-methoxyphenyl-4-phenyl-4,5-dihydro-1,2,4-triazin-3(2H)-one, 4-(2-cyanophenyl)-6-(2-methoxyphenyl)-2-phenyl-4,5-dihydro-1,2,4-triazin-3(2H)-one, 2-(2-cyanophenyl)-6-(2-methoxyphenyl)-4-(2-pyridyl)-4,5-dihydro-1,2,4-triazin-3(2H)-one, 2-(2-cyanophenyl)-4-phenyl-6-(2-pyridyl)-4,5-dihydro-1,2,4-triazin-3(2H)-one, 2-(2-cyanophenyl)-6-(2-pyridyl)-4-(thiophen-3-yl)-4,5-dihydro-1,2,4-triazin-3(2H)-one, 2-(2-cyanophenyl)-6-(2-pyridyl)-4-(2-pyridyl)-4,5-dihydro-1,2,4-triazin-3(2H)-one, 2-(2-cyanophenyl)-6-(2-pyridyl)-4-(3-pyridyl)-4,5-dihydro-1,2,4-triazin-3(2H)-one, 4-(2-cyanophenyl)-2-phenyl-6-(2-pyridyl)-4,5-dihydro-1,2,4-triazin-3(2H)-one, 2-phenyl-6-(2-pyridyl)-4-(thiophen-3-yl)-4,5-dihydro-1,2,4-triazin-3(2H)-one, 2-(2-bromophenyl)-6-(2-pyridyl)-4-(thiophen-3-yl)-4,5-dihydro-1,2,4-triazin-3(2H)-one, 4-(2,4-dimethoxyphenyl)-2-phenyl-6-(2-pyridyl)-4,5-dihydro-1,2,4-triazin-3(2H)-one, 2-(2-bromophenyl)-6-(2-methoxyphenyl)-4-(thiophen-3-yl)-4,5-dihydro-1,2,4-triazin-3(2H)-one, 2-phenyl-6-(2-pyridyl)-4-(3-pyridyl)-4,5-dihydro-1,2,4-triazin-3(2H)-one, 2-(2-bromophenyl)-6-(2-pyridyl)-4-(3-pyridyl)-4,5-dihydro-1,2,4-triazin-3(2H)-one, 2-(2-bromophenyl)-4-(2-cyanophenyl)-6-(2-pyridyl)-4,5-dihydro-1,2,4-triazin-3(2H)-one, 2-(2-bromophenyl)-4,6-diphenyl-4,5-dihydro-1,2,4-triazin-3(2H)-one, 4-(2-bromophenyl)-2,6-diphenyl-4,5-dihydro-1,2,4-triazin-3(2H)-one, 2-(2-bromophenyl)-4-(2-bromophenyl)-6-phenyl-4,5-dihydro-1,2,4-triazin-3(2H)-one, 4-(2-bromophenyl)-6-(2-methoxyphenyl)-2-phenyl-4,5-dihydro-1,2,4-triazin-3(2H)-one, 2-(2-bromophenyl)-4-(2,5-dimethoxyphenyl)-6-(2-methoxyphenyl)-4,5-dihydro-1,2,4-triazin-3(2H)-one, 4-(2,5-dimethoxyphenyl)-6-(2-methoxyphenyl)-2-phenyl-4,5-dihydro-1,2,4-triazin-3(2H)-one, 2-(2-bromophenyl)-6-(2-pyridyl)-4-(2-pyridyl)-4,5-dihydro-1,2,4-triazin-3(2H)-one, 2-phenyl-4-phenyl-6-(2-pyrimidinyl)-4,5-dihydro-1,2,4-triazin-3(2H)-one, 2-(2-bromophenyl)-4-(4-biphenyl)-6-(2-pyridyl)-4,5-dihydro-1,2,4-triazin-3(2H)-one, 2-(2-bromophenyl)-4-(3-nitrophenyl)-6-(2-pyridyl)-4,5-dihydro-1,2,4-triazin-3(2H)-one, 2-(2-bromophenyl)-4-(4-fluorophenyl)-6-(2-pyridyl)-4,5-dihydro-1,2,4-triazin-3(2H)-one, 2-(2-bromophenyl)-4-(3-formylphenyl)-6-(2-pyridyl)-4,5-dihydro-1,2,4-triazin-3(2H)-one, 2-(2-bromophenyl)-4-(3-tolyl)-6-(2-pyridyl)-4,5-dihydro-1,2,4-triazin-3(2H)-one, 2-(2-bromophenyl)-4-(4-thiomethoxyphenyl)-6-(2-pyridyl)-4,5-dihydro-1,2,4-triazin-3(2H)-one, 2-(2-bromophenyl)-4-(2-chloropyridin-5-yl)-6-(2-pyridyl)-4,5-dihydro-1,2,4-triazin-3(2H)-one, 2-(2-cyanophenyl)-4-(3-nitrophenyl)-6-(2-pyridyl)-4,5-dihydro-1,2,4-triazin-3(2H)-one, 2-(2-cyanophenyl)-4-(3-aminophenyl)-6-(2-pyridyl)-4,5-dihydro-1,2,4-triazin-3(2H) and 2-(2-chlorophenyl)-4-phenyl-6-(2-pyrimidinyl)-4,5-dihydro-1,2,4-triazin-3(2H)-one; (40) a pharmaceutical composition comprising a compound represented by formula (I) in the above-mentioned (1), a salt thereof or a hydrate of them as an active ingredient; (41) the pharmaceutical composition according to the above-mentioned (40), which is an inhibitor of an α-amino-3-hydroxy-5-methyl-4-isoxazolepropionic acid (hereinafter referred to as “AMPA”) receptor and/or a kainate receptor; (42) the pharmaceutical composition according to the above-mentioned (40), which is an AMPA receptor inhibitor; (43) the pharmaceutical composition according to the above-mentioned (40), which is a kainate receptor inhibitor; (44) the pharmaceutical composition according to the above-mentioned (40), which is an agent for treating or preventing a disease in which an AMPA receptor or a kainate receptor is participated; (45) the pharmaceutical composition according to the above-mentioned (40), which is an agent for treating or preventing a disease in which this may be “an AMPA” receptor is participated; (46) the pharmaceutical composition according to the above-mentioned (40), which is an agent for treating or preventing an acute neurodegenerative disease; (47) the pharmaceutical composition according to the above-mentioned (40), which is an agent for treating or preventing cerebrovascular disorders at acute stage, head injury, spinal cord injury, neuropathies caused by hypoxia or neuropathies caused by hypoglycemia; (48) the pharmaceutical composition according to the above-mentioned (40), which is an agent for treating or preventing a chronic neurodegenerative disease; (49) the pharmaceutical composition according to the above-mentioned (40), which is an agent for treating or preventing Alzheimer's disease, Parkinson's disease, Huntington's chorea, amyotrophic lateral sclerosis or spinocerebellar degeneration; (50) the pharmaceutical composition according to the above-mentioned (40), which is an agent for treating or preventing epilepsy, hepatic encephalopathy, peripheral neuropathy, Parkinson's syndrome, spasticity, pain, neuralgia, schizophrenia, anxiety, drug abuse, nausea, emesis, dysuria, paropsia caused by glaucoma, paracusis caused by antibiotics, or food poisoning; (51) the pharmaceutical composition according to the above- mentioned (40), which is an agent for treating or preventing infectious encephalomyelitis, cerebrovascular dementia, or dementia or neurosis caused by cerebrospinal meningitis; (52) the pharmaceutical composition according to the above-mentioned (51), wherein the infectious encephalomyelitis is HIV encephalomyelitis; (53) the pharmaceutical composition according to the above-mentioned (40), which is an agent for treating or preventing demyelinating disease; (54) the pharmaceutical composition according to the above-mentioned (53), wherein the demyelinating disease is encephalitis, acute disseminated encephalomyelitis, multiple sclerosis, acute demyelinating polyneuropathy, Guillain-Barre syndrome, chronic inflammatory demyelinating polyneuropathy, Marchifava-Bignami disease, central pontine myelinolysis, neuromyelitis optica, Devic disease, Balo disease, HIV myelopathy, HTLV myelopathy, progressive multifocal leukoencephalopathy or secondary demyelinating disease; (55) the pharmaceutical composition according to the above-mentioned (54), wherein the secondary demyelinating disease is CNS lupus erythematodes, polyarteritis nodosa, Sjoegren's syndrome, sarcoidosis or isolated cerebral vasculitis, etc.

The compound according to the present invention may be a pharmaceutically acceptable salt thereof or a pharmacologically acceptable hydrate thereof.

The pharmaceutical composition of the present invention can contain a pharmacologically acceptable carrier.

The present invention provides a process for treating or preventing a disease in which an AMPA receptor or a kainate receptor is participated, by administering a pharmacologically effective dose of the compound represented by the above formula (I), a salt thereof or a hydrate of them to a patient.

The present invention provides use of the compound represented by the above formula (I), a salt thereof or a hydrate of them for producing an agent for treating or preventing a disease in which an AMPA receptor or a kainate receptor is participated.

Hereinafter, the meanings of symbols, terms, etc. used in this specification are described, and the present invention is described in detail.

As “acute neurodegenerative disease” in the present invention, for example, cerebrovascular disorders at acute stage (subarachnoid hemorrhage, cerebral infarction and the like), head injury, spinal cord injury, and neuropathies due to hypoxia or hypoglycemia; and the like are mentioned. As “chronic neurodegenerative disease”, for example, Alzheimer's disease, Parkinson's disease, Huntington's chorea, amyotrophic lateral sclerosis, spinocerebellar degeneration and the like are mentioned. As “infectious encephalomyelitis”, for example, HIV encephalomyelitis is mentioned, and as “demyelinating disease”, for example, encephalitis, acute disseminated encephalomyelitis, multiple sclerosis, acute dimyelinating polyneuropathy, Guillain-Barre syndrome, chronic inflammatory demyelinating polyneuropathy, Marchifava-Bignami disease, central pontine myelinolysis, neuromyelitis optica, Devic disease, Balo disease, HIV myelopathy, HTLV myelopathy, progressive multifocal leukoencephalopathy, secondary demyelinating disease and the like are mentioned. As “the secondary demyelinating disease” mentioned above, for example, CNS lupus erythematodes, polyarteritis nodosa, Sjoegren's syndrome, sarcoidosis, isolated cerebral vasculitis and the like are mentioned.

Incidentally, in the specification of this application, although structural formula of a compound may express a certain isomer for the sake of convenience, the present invention covers all isomers such as geometrical isomers resulted from the structure of the compound, optical isomers due to asymmetric carbon, rotamers, stereo isomers and tautomers as well as a mixture of isomers and the present invention is not limited to the description of the formula given for the sake of convenience but may be another isomer or may be a mixture. Accordingly, although it is possible that an asymmetric carbon atom is present in a molecule and accordingly that optically active substance and racemic substance may be present, the present invention is not limited thereto but covers any of them. Further, crystal polymorphism may be present but, again, there is no limitation but any of single crystal form or a mixture will do. The compound (I) or its salt related to the present invention may be an anhydride or a hydrate, and either of them are included in the scope of claim for patent in the present invention. The metabolite which is generated by decomposing the compound (I) related to the present invention in vivo, and the prodrug of the compound (I) or its salt related to the present invention produce are also included in the scope of claim for patent in the present invention.

The “halogen atom” used in this specification includes a fluorine atom, chlorine atom, bromine atom and iodine atom, and the atom is preferably a fluorine atom, chlorine atom or bromine atom.

The “C₁₋₆ alkyl group” used in this specification refers to an alkyl group containing 1 to 6 carbon atoms, and preferable examples thereof include linear or branched alkyl groups such as a methyl group, ethyl group, n-propyl group, iso-propyl group, n-butyl group, iso-butyl group, sec-butyl group, tert-butyl group, n-pentyl group, 1,1-dimethylpropyl group, 1,2-dimethylpropyl group, 2,2-dimethylpropyl group, 1-ethylpropyl group, 2-ethylpropyl group, n-hexyl group, 1-methyl-2-ethylpropyl group, 1-ethyl-2-methylpropyl group, 1,1,2-trimethylpropyl group, 1-propylpropyl group, 1-methylbutyl group, 2-methylbutyl group, 1,1,-dimethylbutyl group, 1,2-dimethylbutyl group, 2,2-dimethylbutyl group, 1,3-dimethylbutyl group, 2,3-dimethylbutyl group, 2-ethylbutyl group, 2-methylpentyl group or 3-methylpentyl group.

The “C₂₋₆ alkenyl group” used in this specification refers to an alkenyl group containing 2 to 6 carbon atoms, and preferable examples thereof include a vinyl group, allyl group, 1-propenyl group, 2-propenyl group, isopropenyl group, 2-methyl-1-propenyl group, 3-methyl-1-propenyl group, 2-methyl-2-propenyl group, 3-methyl-2-propenyl group, 1-butenyl group, 2-butenyl group, 3-butenyl group, 1-pentenyl group, 1-hexenyl group, 1,3-hexanedienyl group, 1,6-hexanedienyl group, etc.

The “C₂₋₆ alkynyl group” used in this specification refers to an alkynyl group containing 2 to 6 carbon atoms, and preferable examples thereof include an ethynyl group, 1-propynyl group, 2-propynyl group, 1-butynyl group, 2-butynyl group, 3-butynyl group, 3-methyl-1-propynyl group, 1-ethynyl-2-propynyl group, 2-methyl-3-propynyl group, 1-pentynyl group, 1-hexynyl group, 1,3-hexanediynyl group, 1,6-hexanediynyl group, etc.

The “C₁₋₆ alkoxy group” used in this specification refers to an alkoxy group containing 1 to 6 carbon groups, and preferable examples thereof include a methoxy group, ethoxy group, n-propoxy group, iso-propoxy group, sec-propoxy group, n-butoxy group, iso-butoxy group, sec-butoxy group, tert- butoxy group, n-pentyloxy group, iso-pentyloxy group, sec- pentyloxy group, n-hexoxy group, iso-hexoxy group, 1,1-dimethylpropoxy group, 1,2-dimethylpropoxy group, 2,2-dimethylpropoxy group, 2-ethylpropoxy group, 1-methyl-2-ethylpropoxy group, 1-ethyl-2-methylpropoxy group, 1,1,2-trimethylpropoxy group, 1,1-dimethylbutoxy group, 1,2-dimethylbutoxy group, 2,2-dimethylbutoxy group, 2,3-dimethylbutoxy group, 1,3-dimethylbutoxy group, 2-ethylbutoxy group, 1,3-dimethylbutoxy group, 2-methylpentoxy group, 3-methylpentoxy group, hexyloxygroup, etc.

The “C₂₋₆ alkenyloxy group” used in this specification refers to an alkenyloxy group containing 2 to 6 carbon atoms, and preferable examples thereof include a vinyloxy group, allyloxy group, 1-propenyloxy group, 2-propenyloxy group, isopropenyloxy group, 2-methyl-1-propenyloxy group, 3-methyl-1-propenyloxy group, 2-methyl-2-propenyloxy group, 3-methyl-2-propenyloxy group, 1-butenyloxy group, 2-butenyloxy group, 3-butenyloxy group, 1-pentenyloxy group, 1-hexenyloxy group, 1,3-hexanedienyloxy group, 1,6-hexanedienyloxy group, etc.

The “C₃₋₈ cycloalkyl group” used in this specification refers to a cycloalkyl group containing 3 to 8 carbon atoms, and preferable examples thereof include a cyclopropyl group, cyclobutyl group, cyclopentyl group, cyclohexyl group, cycloheptyl group, cyclooctyl group, etc. The “C₃₋₈ cycloalkane” refers to a cyclic structure corresponding to the above-described “C₃₋₈ cycloalkyl group”, and preferable examples thereof also correspond to examples of the above-described “C₃₋₈ cycloalkyl group”.

The “C₃₋₈ cycloalkenyl group” used in this specification refers to a C₃₋₈ cycloalkenyl group composed of 3 to 8 carbon atoms, and preferable examples thereof include cyclopropen-1-yl, cyclopropen-3-yl, cyclobuten-1-yl, cyclobuten-3-yl, 1,3-cyclobutadien-1-yl, cyclopenten-1-yl, cyclopenten-3-yl, cyclopenten-4-yl, 1,3-cyclopentadien-1-yl, 1,3-cyclopentadien-2-yl, 1,3-cyclopentadien-5-yl, cyclohexen-1-yl, cyclohexen-3-yl, cyclohexen-4-yl, 1,3-cyclohexadien-1-yl, 1,3-cyclohexadien-2-yl, 1,3-cyclohexadien-5-yl, 1,4-cyclohexadien-3-yl, 1,4-cyclohexadien-1-yl, cyclohepten-1-yl, cyclohepten-3-yl, cyclohepten-4-yl, cyclohepten-5-yl, 1,3-cyclohepten-2-yl, 1,3-cyclohepten-1-yl, 1,3-cycloheptadien-5-yl, 1,3-cycloheptadien-6-yl, 1,4-cycloheptadien-3-yl, 1,4-cycloheptadien-2-yl, 1,4-cycloheptadien-1-yl, 1,4-cycloheptadien-6-yl, 1,3,5-cycloheptatrien-3-yl, 1,3,5-cycloheptatrien-2-yl, 1,3,5-cycloheptatrien-1-yl, 1,3,5-cycloheptatrien-7-yl, cycloocten-1-yl, cycloocten-3-yl, cycloocten-4-yl, cycloocten-5-yl, 1,3-cyclooctadien-2-yl, 1,3-cyclooctadien-1-yl, 1,3-cyclooctadien-5-yl, 1,3-cyclooctadien-6-yl, 1,4-cyclooctadien-3-yl, 1,4-cyclooctadien-2-yl, 1,4-cyclooctadien-1-yl, 1,4-cyclooctadien-6-yl, 1,4-cyclooctadien-7-yl, 1,5-cyclooctadien-3-yl, 1,5-cyclooctadien-2-yl, 1,3,5-cyclooctatrien-3-yl, 1,3,5-cyclooctatrien-2-yl, 1,3,5-cyclooctatrien-1-yl, 1,3,5-cyclooctatrien-7-yl, 1,3,6-cyclooctatrien-2-yl, 1,3,6-cyclooctatrien-1-yl, 1,3,6-cyclooctatrien-5-yl, 1,3,6-cyclooctatrien-6-yl group, and the like. The “C₃₋₈ cycloalkene” refers to a cyclic structure corresponding to the above-mentioned “C₃₋₈ cycloalkenyl group”, and preferable examples also correspond to examples of the above-described “C₃₋₈ cycloalkenyl group”.

The “5 to 14 membered non-aromatic heterocyclic group” used in the present invention means a mono-cyclic type, di-cyclic type, or tri-cyclic type 5 to 14 membered non-aromatic heterocyclic group which contains one or more of hetero atoms selected from a group which consists of nitrogen atom, sulfur atom and oxygen atom. Preferable examples in the group include, for example, pyrrolidinyl group, pyrrolinyl group, piperidyl group, piperazinyl group, imidazolidinyl group, pyrazolidinyl group, morpholinyl group, tetrahydrofuryl group, tetrahydropyranyl group, dihydrofuryl group, dihydropyranyl group, imidazolinyl group, oxazolinyl group, and the like. Further, a group derived from a pyridone ring and a non-aromatic condensed ring (for example, a group derived from a phthalimide ring, a succinimide ring, and the like) are also included in the non-aromatic heterocyclic group.

The “C₆₋₁₄ aromatic hydrocarbocyclic group” and the “aryl group” used in the present invention mean an aromatic hydrocarbocyclic group having which is composed of 6 to 14 carbon atoms, and a mono-cyclic group, and a condensed group of a di-cyclic group, a tri-cyclic group and the like are also included. Specific examples in the group include phenyl group, indenyl group, 1-naphthyl group, 2-naphthyl group, azulenyl group, heptalenyl group, biphenyl group, indathenyl group, acenaphthyl group, fluorenyl group, phenalenyl group, phenanthrenyl group, anthracenyl group, cyclopentacyclooctenyl group, benzocyclooctenyl group etc. Further, the “C₆₋₁₄ aromatic hydrocarbon ring” means a cyclic structure corresponding to the above-mentioned “C₆₋₁₄ aromatic hydrocarbon cyclic group”, and preferable examples also correspond to examples of the above-described “C₆₋₁₄ aromatic hydrocarbon cyclic group”.

The “5 to 14 membered aromatic heterocyclic group” and the “heteroaryl group” used in the present invention mean a mono-cyclic type, di-cyclic type, or tri-cyclic type 5 to 14 membered aromatic heterocyclic group which contains one or more of hetero atoms selected from a group which consists of nitrogen atom, sulfur atom and oxygen atom. Preferable examples in the group include aromatic heterocyclic groups containing nitrogen such as pyrrolyl group, pyridyl group, pyridazinyl group, pyrimidinyl group, pyrazinyl group, triazolyl group, tetrazolyl group, benzotriazolyl group, pyrazolyl group, imidazolyl group, benzimidazolyl group, indolyl group, iso- indolyl group, indolizinyl group, prenyl group, indazolyl group, quinolyl group, iso-quinolyl group, quinoliziyl group, phthalazyl group, naphthylidinyl group, quinoxalyl group, quinazolinyl group, cynnolinyl group, pteridinyl group, imidazotriazinyl group, pyrazinopyridazinyl group, acridinyl group, phenanthridinyl group, carbazolyl group, carbazolinyl group, perimidinyl group, phenanthrolinyl group, phenacinyl group, imidazopyridinyl group, imidazopyrimidinyl group, pyrazolopyridinyl group, etc; aromatic heterocyclic groups containing sulfur such as thienyl group or benzothienyl group; aromatic heterocyclic groups containing oxygen such as furyl group, pyranyl group, cyclopentapyranyl group, benzofuryl group or iso-benzofuryl group; and aromatic heterocyclic groups containing 2 or more of different hetero atoms such as thiazolyl group, iso-thiazolyl group, benzothiazolyl group, benzothiadiazolyl group, phenothiazinyl group, isoxazolyl group, furazanyl group, phenoxazinyl group, oxazolyl group, isoxazoyl group, benzoxazolyl group, oxadiazolyl group, pyrazoloxadiazolyl group, imidazothiazolyl group, thienofuranyl group, furopyrrolyl group or pyridoxadinyl group, etc. The “5- to 14-membered aromatic heterocyclic ring” means a cyclic structure corresponding to the above-mentioned “5- to 14-membered aromatic heterocyclic group”, and preferable examples also correspond to examples of the above-described “5- to 14-membered aromatic heterocyclic group”.

The “C₅₋₈ hydrocarbon ring” in this specification refers to a ring selected from C₅₋₈ cycloalkane, C₅₋₈ cycloalkene and C₆₋₈ aromatic hydrocarbon ring. The preferable ring is not particularly limited, and includes the preferable examples of the C₅₋₈ cycloalkane, C₅₋₈ cycloalkene and C₆₋₈ aromatic hydrocarbon ring as defined above.

The “5- to 8-membered heterocyclic ring” in this specification refers to a ring selected from a 5- to 8-membered non-aromatic heterocyclic ring and aromatic heterocyclic ring, and the preferable ring is not particularly limited, and includes the preferable examples of the 5- to 8-membered non-aromatic heterocyclic ring and aromatic heterocyclic ring defined above.

The groups indicated by A¹, A² and A³ in the compound (I) in the present invention indicate independently an optionally substituted C₃₋₈ cycloalkyl group, an optionally substituted C₃₋₈ cycloalkenyl group, an optionally substituted 5 to 14 membered non-aromatic heterocyclic group, an optionally substituted C₆₋₁₄ aromatic hydrocarbocyclic group or an optionally substituted 5 to 14 membered aromatic heterocyclic group, and each of the groups has the same meanings as the above definitions, respectively. The preferable group in A¹, A² and A³ is not specifically limited, but the more preferable group includes phenyl group, pyrrolyl group, pyridyl group, pyridazinyl group, pyrimidinyl group, pyrazinyl group, thienyl group, thiazolyl group, furyl group, naphthyl group, quinolyl group, iso-quinolyl group, indolyl group, benzimidazolyl group, benzothiazolyl group, benzoxazolyl group, imidazopyridyl group, carbazolyl group, cyclopentyl group, cyclohexyl group, cyclohexenyl group, dioxinyl group, adamantyl group, pyrrolidinyl group, piperidyl group, piperazinyl group and morpholinyl group which may be substituted, respectively, etc. The more preferable group includes a group represented by the formula: 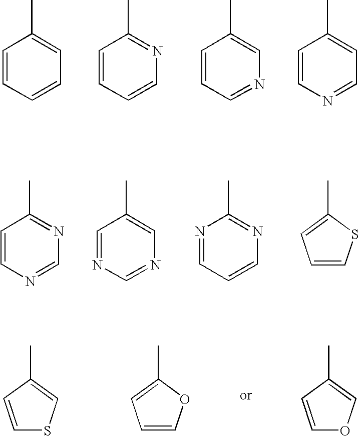
which may optionally have substituents respectively, etc., and the most preferable group includes a group represented by the formula: 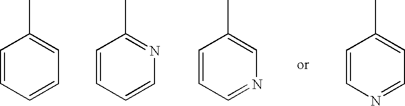
which may optionally have substituents respectively, etc.

Examples of the preferable group in the “substituents” of the groups indicated by A¹, A² and A³ in the compound (I) include a group such as hydroxy group, a halogen atom, nitrile group, nitro group, a C₁₋₆ alkyl group, C₂₋₆ alkenyl group, C₂₋₆ alkynyl group, C₁₋₆ alkoxy group, C₂₋₆ alkenyloxy group, C₂₋₆ alkynyloxy group, C₁₋₆ alkylthio group, C₂₋₆ alkenylthio group, C₂₋₆ alkynylthio group, amino group, a substituted carbonyl group, C₁₋₆ alkylsulfonyl group, C₂₋₆ alkenylsulfonyl group, C₂₋₆ alkynylsulfonyl group, C₁₋₆ alkylsulfinyl group, C₂₋₆ alkenylsulfinyl group, C₂₋₆ alkynylsulfinyl group, formyl group, aralkyl group, heteroarylalkyl group, aralkyloxy group, heteroarylalkyloxy group, C₃₋₈ cycloalkyl group, C₃₋₈ cycloalkenyl group, 5 to 14 membered non-aromatic heterocyclic group, C₆₋₁₄ aromatic hydrocarbon group, 5 to 14 membered aromatic heterocyclic group etc., which may be substituted, respectively.

Examples of the preferable group in the above-mentioned “substituents” of the groups indicated by A¹, A² and A³ include fluorine atom, chlorine atom, bromine atom, iodine atom etc., and the more preferable example includes fluorine atom, chlorine atom and bromine atom. Examples of the preferable group in the “C₁₋₆ alkyl group which may optionally have substituents” include methyl group, ethyl group, n-propyl group, iso-propyl group, n-butyl group, iso-butyl group, tert-butyl group, n-pentyl group, iso-pentyl group, neopentyl group, n-hexyl group, 1-methylpropyl group, 1,2-dimethylpropyl group, 2-ethylpropyl group, 1-methyl-2-ethylpropyl group, 1-ethyl-2-methylpropyl group, 1,1,2-trimethylpropyl group, 1-methylbutyl group, 2-methylbutyl group, 1,1-dimethylbutyl group, 2,2-dimethylbutyl group, 2-ethylbutyl group, 1,3-dimethylbutyl group, 2-methylpentyl group, 3-methylpentyl group etc, each of which may be substituted. Examples of the preferable group in the “C₂₋₆ alkenyl group which may optionally have substituents” include a vinyl group, allyl group, 1-propenyl group, iso-propenyl group, 1-buten-1-yl group, 1-buten-2-yl group, 1-buten-3-yl group, 2-buten-1-yl group, 2-buten-2-yl group etc., each of which may be substituted. Examples of the preferable group in the “C₂₋₆ alkynyl group which may optionally have substituents respectively” include an ethynyl group, 1-propynyl group, 2-propynyl group, butynyl group, pentynyl group, hexynyl group etc., each of which may be substituted. Further, preferable examples of the “substituents” in the “which may optionally have substituents” include 1 or more groups selected from hydroxy group, nitrile group, a halogen atom, an N—C₁₋₆ alkylamino group, an N,N-di-C₁₋₆ alkylamino group, an N—C₂₋₆ alkenylamino group, an N,N-di-C₂₋₆ alkenylamino group, an N—C₂₋₆ alkynylamino group, an N,N-di-C₂₋₆ alkynylamino group, a C₆₋₁₄ aromatic hydrocarbocyclic group (for example, phenyl group etc.), a 5 to 14 membered aromatic heterocyclic group (for example, thienyl group, furyl group, pyridyl group, pyridazinyl group, pyrimidinyl group, pyrazinyl group etc.), an aralkyloxy group, a heteroaryloxy group, a TBDMS-oxy group, a C₁₋₆ alkylsulfonylamino group, a C₂₋₆ alkenylsulfonylamino group, a C₂₋₆ alkynylsulfonylamino group, a C₁₋₆ alkylcarbonyloxy group, a C₂₋₆ alkenylcarbonyloxy group, a C₂₋₆ alkynylcarbonyloxy group, a C₁₋₆ alkylcarbamoyl group, a C₂₋₆ alkenylcarbamoyl group, a C₂₋₆ alkynylcarbamoyl group, and the like.

Preferable examples in the “C₁₋₆ alkoxy group which may optionally have substituents” include methoxy group, ethoxy group, n-propoxy group, iso-propoxy group, sec-propoxy group, n-butoxy group, iso-butoxy group, sec-butoxy group, tert-butoxy group, n-pentoxy group, iso-pentoxy group, sec-pentoxy group, tert-pentoxy group, n-hexoxy group, iso-hexoxy group, 1,2-dimethylpropoxy group, 2-ethylpropoxy group, 1-methyl-2-ethylpropoxy group, 1-ethyl-2-methylpropoxy group, 1,1,2-trimethylpropoxy group, 1,1-dimethylbutoxy group, 2,2-dimethylbutoxy group, 2-ethylbutoxy group, 1,3-dimethylbutoxy group, 2-methylpentoxy group, 3-methylpentoxy group, hexyloxy group etc. Preferable examples in the “C₂₋₆ alkenyloxy group which may optionally have substituents” include vinyloxy group, allyloxy group, 1-propenyloxy group, iso-propenyloxy group, 1-buten-1-yloxy group, 1-buten-2-yloxy group, 1-buten-3-yloxy group, 2-buten-1-yloxy group, 2-buten-2-yloxy group etc. Preferable examples in the “C₂₋₆ alkynyloxy group which may optionally have substituents” include ethynyloxy group, 1-propynyloxy group, 2-propynyloxy group, butynyloxy group, pentynyloxy group, hexynyloxy group etc. Further, preferable examples of the “substituents” in the “which may optionally have substituents” include 1 or more groups selected from an C₁₋₆ alkylamino group, an aralkyloxy group, hydroxy group, and the like.

Respectively preferable examples in the “C₁₋₆ alkylthio group which may optionally have substituents”, “C₂₋₆ alkenylthio group which may optionally have substituents” and “C₂₋₆ alkynylthio group which may optionally have substituents” include a C₁₋₆ alkylthio group (for example, methylthio group, ethylthio group, n-propylthio group, iso-propylthio group, n-butylthio group, iso-butylthio group, tert-butylthio group, n-pentylthio group, iso-pentylthio group, neopentylthio group, n-hexylthio group etc.), a C₂₋₆ alkenylthio group (for example, vinylthio group, allylthio group, 1-propenylthio group, iso-propenylthio group, 1-buten-1-ylthio group, 1-buten-2-ylthio group, 1-buten-3-ylthio group, 2-buten-1-ylthio group, 2-buten-2-ylthio group etc.) and a C₂₋₆ alkynylthio group (for example, ethynylthio group, 1-propynylthio group, 2-propynylthio group, butynylthio group, pentynylthio group, hexynylthio group etc.), which may be optionally substituted by 1 or more groups selected from the group consisting of hydroxy group, a halogen atom, nitrile group and nitro group.

Preferable examples in the “carbonyl group which was substituted” include a group which is represented by the formula —CO—W (examples of W in the formula include a C₁₋₆ alkyl group, a C₂₋₆ alkenyl group, a C₂₋₆ alkynyl group, a C₁₋₆ alkoxy group, amino group, an N—C₁₋₆ alkylamino group, an N,N-di(C₁₋₆ alkyl)amino group, an N—C₂₋₆ alkenylamino group, an N,N-di(C₂₋₆ alkenyl)amino group, an N—C₂₋₆ alkynylamino group, an N,N-di(C₂₋₆ alkynyl)amino group, an N—C₁₋₆ alkyl-N—C₂₋₆ alkenylamino group, an N—C₁₋₆ alkyl-N—C₂₋₆ alkynylamino group, an N—C₂₋₆ alkenyl-N—C₂₋₆ alkynylamino group etc.).

Examples of the “substituents” in the “amino group which may optionally have substituents” include 1 or 2 groups selected from a C₁₋₆ alkyl group, C₂₋₆ alkenyl group, C₂₋₆ alkynyl group, C₁₋₆ alkylsulfonyl group, C₂₋₆ alkenylsulfonyl group, C₂₋₆ alkynylsulfonyl group, C₁₋₆ alkylcarbonyl group, C₂₋₆ alkenylcarbonyl group, C₂₋₆ alkynylcarbonyl group etc., which may be substituted, respectively. Preferable examples in the “substituents” of the C₁₋₆ alkyl group, C₂₋₆ alkenyl group, C₂₋₆ alkynyl group, C₁₋₆ alkylsulfonyl group, C₂₋₆ alkenylsulfonyl group, C₂₋₆ alkynylsulfonyl group, C₁₋₆ alkylcarbonyl group, C₂₋₆ alkenylcarbonyl group and C₂₋₆ alkynylcarbonyl group include hydroxy group, a halogen atom, nitrile group, a C₁₋₆ alkoxy group, a C₁₋₆ alkylthio group etc. Specifically preferable examples in the “amino group which may optionally have substituents” in particular include methylamino group, ethylamino group, n-propylamino group, iso-propylamino group, n-butylamino group, iso-butylamino group, tert-butylamino group, n-pentylamino group, iso-pentylamino group, neopentylamino group, n-hexylamino group, 1-methylpropylamino group, 1,2-dimethylpropylamino group, 2-ethylpropylamino group, 1-methyl-2-ethylpropylamino group, 1-ethyl-2-methylpropylamino group, 1,1,2-trimethylpropylamino group, 1-methylbutylamino group, 2-methylbutylamino group, 1,1-dimethylbutylamino group, 2,2-dimethylbutylamino group, 2-ethylbutylamino group, 1,3-dimethylbutylamino group, 2-methylpentylamino group, 3-methylpentylamino group, N,N-dimethylamino group, N,N- diethylamino group, N,N-di(n-propyl)amino group, N,N-di(iso-propyl)amino group, N,N-di(n-butyl)amino group, N,N-di(iso-butyl)amino group, N,N-di(tert-butyl)amino group, N,N-di(n-pentyl)amino group, N,N-di(iso-pentyl)amino group, N,N-di(neopentyl)amino group, N,N-di(n-hexyl)amino group, N,N-di(1-methylpropyl)amino group, N,N-di(1,2-dimethylpropyl)amino group, N-methyl-N-ethylamino group, N-ethyl-N-(n-propyl)amino group, N-methyl-N-(iso-propyl)amino group, vinylamino group, allylamino group, (1-propenyl)amino group, iso-propenylamino group, (1-buten-1-yl)amino group, (1-buten-2-yl)amino group, (1-buten-3-yl)amino group, (2-buten-1-yl)amino group, (2-buten-2-yl)amino group, N,N-divinylamino group, N,N-diallylamino group, N,N-di(1-propenyl)amino group, N,N-di(iso-propenyl)amino group, N-vinyl-N-allylamino group, ethynylamino group, 1-propynylamino group, 2-propynylamino group, butynylamino group, pentynylamino group, hexynylamino group, N,N-diethynylamino group, N,N-di(1-propynyl)amino group, N,N-di(2-propynyl)amino group, N,N-dibutynylamino group, N,N-dipentynylamino group, N,N-dihexynylamino group, hydroxymethylamino group, 1-hydroxyethylamino group, 2-hydroxyethylamino group, 3-hydroxy-n-propylamino group, methylsulfonylamino group, ethylsulfonylamino group, n-propylsulfonylamino group, iso-propylsulfonylamino group, n-butylsulfonylamino group, tert-butylsulfonylamino group, vinylsulfonylamino group, allylsulfonylamino group, iso-propenylsulfonylamino group, iso-pentenylsulfonylamino group, ethynylsulfonylamino group, methylcarbonylamino group, ethylcarbonylamino group, n-propylcarbonylamino group, iso-propylcarbonylamino group, n-butylcarbonylamino group, tert-butylcarbonylamino group, vinylcarbonylamino group, allylcarbonylamino group, iso-propenylcarbonylamino group, iso-pentenylcarbonylamino group, ethynylcarbonylamino group etc.

Respectively preferable examples in the “C₁₋₆ alkylsulfonyl group which may optionally have substituents”, “C₁₋₆ alkenylsulfonyl group which may optionally have substituents”, “C₂₋₆ alkynylsulfonyl group which may optionally have substituents”, “C₁₋₆ alkylsulfinyl group which may optionally have substituents”, “C₂₋₆ alkenylsulfinyl group which may optionally have substituents” and “C₁₋₆ alkynylsulfinyl group which may optionally have substituents” include methylsulfonyl group, ethylsulfonyl group, n-propylsulfonyl group, iso-propylsulfonyl group, n-butylsulfonyl group, tert-butylsulfonyl group, vinylsulfonyl group, allylsulfonyl group, iso-propenylsulfonyl group, iso-pentenylsulfonyl group, ethynylsulfonyl group, methylsulfinyl group, ethylsulfinyl group, n-propylsulfinyl group, iso-propylsulfinyl group, n-butylsulfinyl group, tert-butylsulfinyl group, vinylsulfinyl group, allylsulfinyl group, iso-propenylsulfinyl group, iso-pentenylsulfinyl group, ethynylsulfinyl group etc.

Preferable examples in the “aralkyl group” and “heteroarylalkyl group” include benzyl group, phenethyl group, naphthylmethyl group, naphthylethyl group, pyridylmethyl group, pyridylethyl group, thienylmethyl group, thienylethyl group etc., preferable examples in the “aralkyloxy group” include benzyloxy group, phenethyloxy group, phenylpropoxy group, naphthylmethyloxy group, naphthylethyloxy group, naphthylpropyloxy group etc., and preferable examples in the “heteroarylalkyloxy group” include pyridylmethyloxy group, pyrazinylmethyloxy group, pyrimidinylmethyloxy group, pyrrolylmethyloxy group, imidazolylmethyloxy group, pyrazolylmethyloxy group, quinolylmethyloxy group, iso-quinolylmethyloxy group, furfuryloxy group, thienylmethyloxy group, thiazolylmethyloxy group etc.

Preferable examples in the “C₃₋₈ cycloalkyl group which may optionally have substituents” and “C₃₋₈ cycloalkenyl group which may optionally have a substituent” include a C₃₋₈ cycloalkyl group (for example, cyclopropyl group, cyclobutyl group, cyclopentyl group, cyclohexyl group, cycloheptyl group, and the like) and a C₃₋₈ cycloalkenyl group (for example, cyclopropenyl group, cyclobutenyl group, cyclopentenyl group, cyclohexenyl group, cycloheptenyl group, and the like) which may be optionally substituted respectively by 1 or more groups selected from hydroxy group, a halogen atom, nitrile group, a C₃₋₈ alkyl group (for example, methyl group, ethyl group, n-propyl group, iso-propyl group, n-butyl group, iso-butyl group, tert-butyl group, n-pentyl group, iso-pentyl group, neopentyl group, n-hexyl group etc.), a C₁₋₆ alkoxy group (for example, methoxy group, ethoxy group, n-propoxy group, iso-propoxy group, sec-propoxy group, n-butoxy group, iso-butoxy group, sec-butoxy group, tert-butoxy group, n-pentoxy group, iso-pentoxy group, sec-pentoxy group, tert-pentoxy group, n-hexoxy group etc.), a C₁₋₆ alkoxy C₁₋₆ alkyl group, an aralkyl group (for example, benzyl group, phenethyl group, naphthylmethyl group, naphthylethyl group etc.), and the like.

Preferable examples of the “5 to 14 membered non-aromatic heterocyclic group”, “C₆₋₁₄ aromatic hydrocarbocyclic group” and “5 to 14 membered aromatic heterocyclic group” in “an optionally substituted 5 to 14 membered non-aromatic heterocyclic group”, “an optionally substituted C₆₋₁₄ aromatic hydrocarbocyclic group” and “an optionally substituted 5 to 14 membered aromatic heterocyclic group” are not specifically limited, but the more preferable “5 to 14 membered non-aromatic heterocyclic group” includes pyrrolidinyl group, pyrrolinyl group, piperidinyl group, piperazinyl group, imidazolinyl group, pyrazolyl group, imidazolidinyl group, morpholinyl group, phthalimidoyl group, a succinimidoyl group etc.; the more preferable “C₆₋₁₄ aromatic hydrocarbocyclic group” includes phenyl group, indenyl group, naphthyl group, azulenyl group, heptalenyl group, biphenyl group etc.; the more preferable “5 to 14 membered aromatic heterocyclic group” includes pyrrolyl group, pyridyl group, pyridazinyl group, pyrimidinyl group, pyrazinyl group, pyrazolyl group, imidazolyl group, thienyl group, furyl group, thiazolyl group, iso-thiazolyl group, quinolyl group, iso-quinolyl group, indolyl group, benzimidazolyl group, benzothiazolyl group, benzoxazolyl group, carbazolyl group, dioxinyl group etc., respectively. Further, preferable examples of the “substituents” in the “which may optionally have substituents” include 1 or more groups selected from hydroxy group, a halogen atom (for example, fluorine atom, chlorine atom, bromine atom, iodine atom etc.), nitrile group, a C₁₋₆ alkyl group (for example, methyl group, ethyl group, n-propyl group, iso-propyl group, n-butyl group, iso-butyl group, tert-butyl group, n-pentyl group, iso-pentyl group, neopentyl group, n-hexyl group etc.), a C₁₋₆ alkoxy group (methoxy group, ethoxy group, n-propoxy group, iso-propoxy group, sec-propoxy group, n-butoxy group, iso-butoxy group, sec-butoxy group, tert-butoxy group, n-pentoxy group, iso-pentoxy group, sec-pentoxy group, tert-pentoxy group, n-hexoxy group etc.), a C₁₋₆alkoxy C₁₋₆ alkyl group (for example, methoxymethyl group, methoxyethyl group, ethoxymethyl group, ethoxyethyl group etc.), an aralkyl group (for example, benzyl group, phenethyl group, naphthylmethyl group, naphthylethyl group etc.), and the like. Further, an amino group, a cyclic amino group, and an alkoxyamino group which may optionally have substituents are also preferable as the substituents.

In the compound (I), Q represents O (oxygen atom), S (sulfur atom) or NH. O is most preferred.

In the compound (I), Z represents C (carbon atom) or N (nitrogen atom).

In the compound (I), when Z is N, R¹ as a substituent group is absent. In this case, R¹ represents a lone pair of N.

X¹, X² and X³ are independent of each other and each represents a single bond, an optionally substituted C₁₋₆ alkylene group, an optionally substituted C₂₋₆ alkenylene group, an optionally substituted C₂ alkynylene group, —NH—, —O—, —N(R⁴)CO—, —CON(R⁵)—, —N(R⁶)CH₂—, —CH₂N(R⁷)—, —CH₂CO—, —COCH₂—, —N(R⁸)SO₀₋₂—, —SO₀₋₂N(R⁹)—, —CH₂SO₀₋₂—, —SO₀₋₂CH₂—, —CH₂O —, —OCH₂—, —N(R¹⁰)CON(R¹)—, —N(R¹²)CS—N(R¹³)— or —SO₀₋₂—. In these formula, R⁴, R⁵, R⁶, R⁷, R⁸, R⁹, R¹⁰, R¹¹, R¹² and R¹³ are independent of each other and each represents a hydrogen atom, a C₁₋₆ alkyl group or a C₁₋₆ alkoxy group, and the “C₁₋₆ alkyl group” is preferably a methyl group, ethyl group, n-propyl group, i-propyl group, n-butyl group or tert-butyl group, and the “C₁₋₆ alkoxy group” is preferably a methoxy group, ethoxy group, n-propoxy group, i-propoxy group, n-butoxy group, tert-butyloxy group or the like.

In the formula above, —SO₀₋₂— means that S as a linking chain has 0, 1 or 2 oxygen atoms, and specifically —SO₀₋₂— is —S—, —SO— or —SO₂—.

The “C₁₋₆ alkylene group”, “C₂₋₆ alkenylene group” and “C₂₋₆ alkynylene group” described above refer respectively to linking chains corresponding to the “C₁₋₆ alkyl group”, “C₁₋₆ alkenyl group” and “C₂₋₆ alkynyl group” described above, and preferable examples thereof include —CH₂—, —CH₂)₂—, —CH(CH₃)—, —(CH₂)₃—, —CH(CH₃)—CH₂—, —CH₂CH(CH₃)—, —CH═CH—, —CH═CHCH₂—, —CH₂CH=CH—, —C(CH₃)═CH—, —CH═C(CH₃)—, —C≡C—, —C≡CCH₂—, —CH₂C≡—C—, etc., and more preferable examples include —CH₂—, —(CH₂)₂—, —(CH₂)₃—, —CH═CH—, —CH═CHCH₂—, —CH₂CH═CH—, —C≡C—, —C≡CCH₂—, —CH₂C≡C—, etc. Preferable examples of the “substituent groups” on the “optionally substituted C₁₋₃ alkylene group”, “optionally substituted C₂₋₃ alkenylene group” and “optionally substituted C₂₋₃ alkynylene group” include a halogen atom (for example, a fluorine atom, chlorine atom, bromine atom, etc.), a hydroxyl group, a nitrile group, a nitro group, etc. Preferable examples of the “optionally substituted C₁₋₃ alkylene group”, “optionally substituted C₂₋₃ alkenylene group” and “optionally substituted C₂₋₃ alkynylene group” include —CH₂—, —CH(OH)—, —CH(CN)—, —CH₂CH₂—, —CH(OH)CH₂—, —CH(CN)CH₂—, —CH₂CH(OH)—, —CH₂CH(CN)—, —CH═CH—, —CH═CHCH₂—, —CH═CHCH(OH)—, —CH═CHCH(CN)—, —CH(OH)CH═CH—, —CH(CN)CH═CH—, —C≡C—, etc.

Preferable examples of X¹, X² and X³ include a single bond, —CH₂—, —CH(OH)—, —CH(CN)—, —CH₂CH₂—, —CH(OH)CH₂—, —CH(CN)CH₂—, —CH₂CH(OH)—, —CH₂CH(CN)—, —CH═CH—, —CH═CHCH₂—, —CH═CHCH(OH)—, —CH═CHCH(CN)—, —CH(OH)CH═CH—, —CH(CN)CH═CH—, —C≡C— and —NHCONH—. More preferable examples include a single bond, —CH₂—, —CH(OH)—, —CH(CN)—, —CH₂CH₂—, —CH(OH)CH₂—, —CH(CN)CH₂—, —CH₂CH(OH)—, —CH₂CH(CN)—, —CH═CH— and —C≡C—, further preferable examples include a single bond, —CH₂— and —CH(OH)—, and the most preferable example is a single bond.

In the compound (I), (1) when Z is C, R¹ and R² independently represents a hydrogen atom, an optionally substituted C₁₋₆ alkyl group, an optionally substituted C₂₋₆ alkenyl group or an optionally substituted C₂₋₆ alkynyl group, or R¹ and R² may be bound to each other such that the partial structure CR¹—CR² forms a carbon-carbon double bond, that is, the structure represented by C═C. Further, (2) when Z is N, R¹ represents a lone pair; and R² represents a hydrogen atom, an optionally substituted C₁₋₆ alkyl group, an optionally substituted C₂₋₆ alkenyl group or an optionally substituted C₂₋₆ alkynyl group.

The phrase “optionally substituted” in the “optionally substituted C₁₋₆ alkyl group”, “optionally substituted C₂₋₆ alkenyl group” and “optionally substituted C₂₋₆ alkynyl group” mean that these group may be substituted with at least one group selected from a hydroxyl group, a thiol group, a nitrile group, a halogen atom (for example, a fluorine atom, chlorine atom, bromine atom, iodine atom, etc.), a nitro group, an amino group, a C₁₋₆ alkylamino group, a di-C₁₋₆ alkyl-amino group, a C₂₋₆ alkenylamino group, a di-C₂₋₆ alkenyl-amino group, a C₂₋₆ alkynylamino group, a di-C₂₋₆ alkynyl-amino group, a C₆₋₁₄ aromatic hydrocarbon group (for example, a phenyl group etc.), a 5- to 14-membered aromatic heterocyclic group (for example, a thienyl group, furyl group, pyridinyl group, pyridazyl group, pyrimidinyl group, pyrazinyl group, etc.), an aralkyloxy group, a heteroaryloxy group, a TBDMS oxy group, a C₁₋₆ alkylsulfonylamino group, a C₂ alkenylsulfonylamino group, a C₂ alkynylsulfonylamino group, a C₁₋₆ alkylcarbonyloxy group, a C₂₋₆ alkenylcarbonyloxy group, a C₂ alkynylcarbonyloxy group, a C₂₋₆ alkylcarbamoyl group, a C₂₋₆ alkenylcarbamoyl group and a C₂₋₆ alkynylcarbamoyl group, more preferably with substituent groups such as a hydroxyl group, nitrile group, halogen atom, nitro group and amino group. The “C₁₋₆ alkyl group”, “C₂₋₆ alkenyl group” and “C₂₋₆ alkynyl group” have the same meanings as defined above.

In the compound (I), R³ represents a hydrogen atom, an optionally substituted C₁₋₆ alkyl group, an optionally substituted C₂₋₆ alkenyl group or an optionally substituted C₂₋₆ alkynyl group, or may be bound to any atom in A¹ or A³ to form, together with the atom, an optionally substituted C₅₋₈ hydrocarbon ring or a 5- to 8-membered heterocyclic ring. However, when Z is N, each of X¹, X² and X³is a single bond, and each of A¹, A² and A³ is a phenyl group, when Z is N, each of X¹, X² and X³ is a single bond, A¹ is an o,p-dimethylphenyl group, A² is an o-methylphenyl group and A³ is a phenyl group, or when Z is N, each of X¹, X² and X³ is a single bond, A¹ is an o-methylphenyl group, A² is a p-methoxyphenyl group and A³ is a phenyl group, at least one of R² and R³ is a group which is not a hydrogen atom.

The “optionally substituted C₁₋₆ alkyl group”, “optionally substituted C₂₋₆ alkenyl group” and “optionally substituted C₂₋₆ alkynyl group” have the same meanings as defined for R¹ and R². In the “optionally substituted C₅₋₈ hydrocarbon ring” and “optionally substituted 5- to 8-membered heterocyclic ring”, the “C₅₋₈ hydrocarbon ring” and “5- to 8-membered heterocyclic ring” have the same meanings as defined above, and the meanings of substituent groups on the “C₅₋₈ hydrocarbon ring” and “5- to 8-membered heterocyclic ring” are identical with the meanings of substituent groups on A¹, A² and A³.

In the “optionally substituted C₅₋₈ hydrocarbon ring” or “optionally substituted 5- to 8-membered heterocyclic ring” formed by R³ together with any atom in A¹ or A³, to which R³ is bound, preferable mode is ring B or C in compounds represented by the formula: 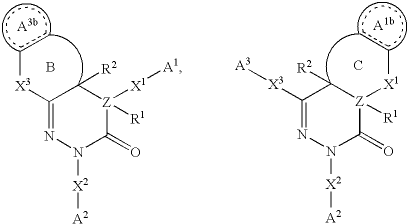
wherein each symbol has the same meaning as defined above. More preferable embodiment is ring B or C in compounds represented by the formula: 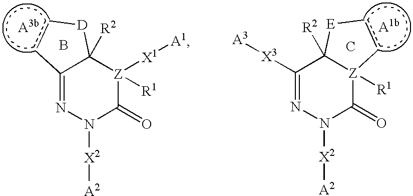
wherein D and E each represent —CH₂—, —CH₂)₂—, —C═C—, —C═C—, —O—, —CH₂—, —CH₂O—, —SO₀₋₂—, —SCH₂—, —CH₂S—, —SOCH₂—, —CH₂SO—, —SO₂CH₂—, —CH₂SO₂—, —NR¹⁴—, —NR¹⁴CH₂— or —CH₂NR¹⁴— (wherein R¹⁴ representsa hydrogen atom, a C₁₋₆ alkyl group, an optionally substituted C₃₋₈ cycloalkyl group, an optionally substituted 5- to 14-membered non-aromatic heterocyclic group, an optionally substituted C₆₋₁₄ aromatic hydrocarbon cyclic group or an optionally substituted 5- to 14-membered aromatic heterocyclic group), and the substitutable positions in D and E may be substituted; and other symbols have the same meanings as defined above. Further preferable embodiment is the case where D or E is —H₂—, —CH₂)₂—, —O—, —S—, —SO—, —SO₂—, —NH¹⁴—, —O—CH₂—, —CH₂—O—, —S—CH₂—, CH₂—S—, —SO—CH₂—, —CH₂—SO—, —SO₂—CH₂— or —CH₂—SO₂—, and the most preferable embodiment is the case where D or E is —CH₂—, —O—, —S—, —SO— or —SO₂— wherein R¹⁴ have the same meaning as defined above.

In the compound (I), when R³ is bound to any atom in A¹ or A³ to form a ring with the atom, the ring may further have one or more substituent groups. Preferable examples of such substituent groups include a hydroxyl group, a halogen atom, a cyano group and a nitro group, an optionally substituted C₁₋₆ alkyl group, C₂₋₆ alkenyl group, C₂₋₆ alkynyl group, C₁₋₆ alkoxy group, C₂₋₆ alkenyloxy group, C₁₋₆ alkylthio group, C₂₋₆ alkenylthio group, amino group, etc.

The mode of the compound (1) in this invention is not particularly limited, and the respective groups can be arbitrarily combined easily by those skilled in the art, and the compound (I) in a preferable embodiment is a compound wherein A¹, A² and A³ independently represents a C₆₋₁₄ aromatic hydrocarbon group or a 5- to 14-membered aromatic heterocyclic group, each of which may be substituted, and in a more preferable embodiment, it is a compound wherein Q is O, that is, a compound represented by the formula: 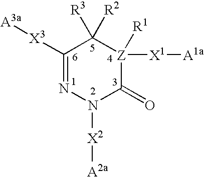
wherein rings A^{1a}, A^{2a} and A^{3a} are independent of each other and each represents a C₆₋₁₄ aromatic hydrocarbon cyclic group or a 5- to 14-membered aromatic heterocyclic group, each of which may be substituted; and X¹, X², X³, Z, R¹, R² and R³ have the same meanings as defined above. Further preferable embodiment is a compound wherein A¹, A² and A³ are independent of each other and each represents a C₆₋₁₄ aromatic hydrocarbon cyclic group or a 5- to 14-membered aromatic heterocyclic group, each of which may be substituted; Q is O; and each of X¹, X² and X³ is a single bond, that is, a compound represented by the formula:

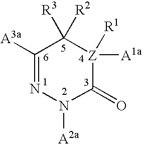

There is no particular limitation for “a salt” in the specification of the present application so far as it forms a salt with the compound of the present invention and is a pharmacologically acceptable one. Preferably, salt with a hydrogen halide (such as hydrofluoride, hydrochloride, hydrobromide or hydroiodide), salt with an inorganic acid (such as sulfate, nitrate, perchlorate, phosphate, carbonate or bicarbonate), salt with an organic carboxylic acid (such as acetate, trifluoroacetate, oxalate, maleate, tartrate, fumarate or citrate), salt with an organic sulfonic acid (such as methanesulfonate, trifluoromethanesulfonate, ethanesulfonate, benzenesulfonate, toluenesulfonate or camphor-sulfonate), salt with an amino acid (such as aspartate or glutamate), salt with a quaternary amine, salt with an alkaline metal (such as sodium salt or potassium salt) and salt with an alkaline earth metal (such as magnesium salt or calcium salt). More preferred examples of the “pharmacologically acceptable salt” are hydrochloride, oxalate etc.

Compound (I) according to the present invention can be produced by a known method or its analogous method. Typical production methods are shown below. The “room temperature” mentioned in the following typical production methods, Reference Examples and Examples refers to 0 to about 40° C.

As the compound according to the present invention which is represented by the above formula (I), the compounds represented by the following formula (I-1) or (III) wherein Z is a carbon atom can be produced by condensing a ketocarboxylic acid derivative (i) or a ketocarboxylate derivative (iii) with a substituted hydrazine derivative (ii) or (ii)′, as shown in the reaction scheme:
Production Method 1-a 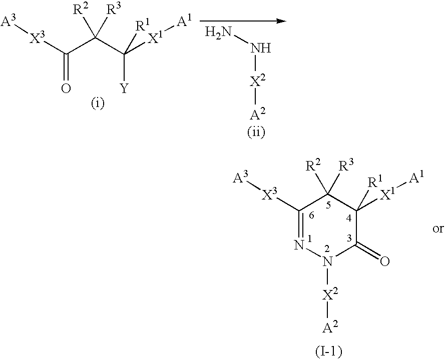
Production Method 1-b 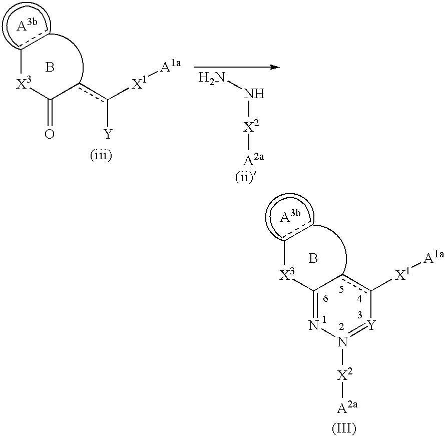
wherein X¹, X², X³, A¹, A², A³, A^{1a}, A^{2a}, A^{3b}, B, R¹, R² and R³ have the same meanings as defined above; and Y represents a carboxylic acid or an ester group. This reaction is conducted preferably in the presence of a solvent from viewpoints of operativeness and stirring. The solvent varies depending on the starting material, reagents etc., and is not particularly limited insofar as it is inert to the reaction and dissolves the starting material to a certain degree, and preferable examples include ethanol, toluene, xylene, acetic acid, etc. The substituted hydrazine derivative used varies depending on the starting material, the solvent used, reaction temperature etc., and is not particularly limited insofar as it is inert to the reaction. From viewpoints of stability and availability, the hydrazine derivative is preferably a hydrochloride. The reaction is carried out usually at room temperature or by heating under reflux, preferably at 50 to 120° C., Though, the reaction temperature varies depending on the starting material used, reagents etc., and is not particularly limited. In this reaction, an acid catalyst such as p-toluene sulfonic acid or camphor sulfonic acid can be added as an additive to give good results such as reduction in reaction time, improvement in yield, etc.

Among the compounds according to the present invention which is represented by the above formula (I), the compounds (the following formula (1-2) or (III-1)) wherein Z is C; X² is a single bond; and A² is an optionally substituted aromatic ring or an optionally substituted heterocyclic ring can be produced by introducing a substituent group to the 2-position of a pyridazinone derivative (iv) or (v) synthesized by condensation reaction of the ketocarboxylic acid derivative (i) or ketocarboxylate derivative (iii) with hydrazine, as shown in the reaction scheme:
Production Method 2-a 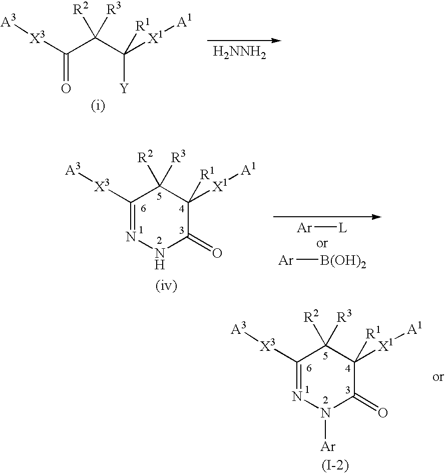
Production Method 2-b 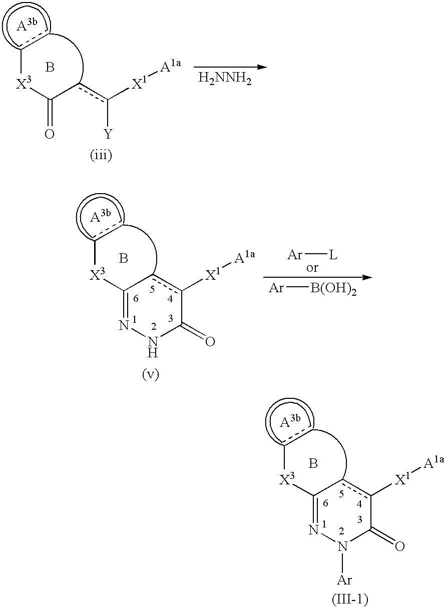
wherein X¹, X³, A¹, A³, A^{1a}, A^{3b}, B, R¹, R² and R³ have the same meanings as defined above; Y represents a carboxylic acid or an ester group; Ar represents an aromatic hydrocarbon ring or an aromatic heterocyclic group, each of which may be substituted; and L represents a bromine atom or an iodine atom. The condensation reaction in this reaction is conducted preferably in the presence of a solvent from viewpoints of operativeness and stirring. The solvent varies depending on the starting material, reagents etc., and is not particularly limited insofar as it is inert to the reaction and dissolves the starting material to a certain degree, and preferable examples include ethanol, toluene, xylene, etc. The preferable examples of hydrazine used includes hydrazine anhydride, hydrazine hydrate, hydrazine hydrochloride etc., and it varies depending on the solvent used etc., and is not particularly limited. The reaction temperature varies depending on the starting material, the solvent used etc., and is not particularly limited, but usually the reaction is carried out at room temperature or by heating under reflux, preferably at 40 to 120° C.

The method of introducing a substituent group into the 2-position of the pyridazinone derivative (iv) or (v) involves, for example, Ullman reaction with a halogen aryl derivative (Ar-L in the reaction scheme above) in order to introduce an aryl group. The reaction conditions are not particularly limited, but the reaction is carried out typically and preferably in the presence of copper, copper bromide, copper iodide, etc. with a base such as potassium carbonate, sodium carbonate, potassium acetate, sodium acetate, etc. in a solvent under stirring. The solvent used in the above Ullman reaction varies depending on the starting material, reagents etc., and is not particularly limited insofar as it is inert to the reaction and dissolves the starting material to a certain degree. The preferable examples thereof include dimethylformamide, dichlorobenzene, nitrobenzene, amyl alcohol etc. The reaction temperature varies depending on the starting material used, the solvent used etc., and is not particularly limited. Preferably the reaction is carried out by heating under reflux. At such temperature, the reaction can be finished in a short time and a good result is given.

An alternative method of introducing the substituent group to the 2-position of the pyridazinone derivative (iv) or (v) is a method of coupling the pyridazinone derivative (iv) or (v) with an aryl boronic acid derivative (Ar—B(OH)₂ in the reaction scheme above) in the presence of a base with a copper compound. As the arylboronic acid derivative used, for example, an optionally substituted phenylboronic acid derivative and an optionally substituted heterocyclic boronic acid derivative are preferred. The base used varies depending on the starting material, the solvent used etc., and is not particularly limited insofar as it is inert to the reaction. The preferable examples include triethylamine, pyridine, tetramethylethylenediamine, etc. Further, the preferable examples of the copper compound used include, for example, copper acetate, di-μ-hydroxo-bis[(N,N,N′,N′-tetramethylethylenediamine)copper (II)]chlorid etc. This coupling reaction is conducted preferably in the presence of a solvent, and the solvent varies depending on the starting material, reagents etc., and is not particularly limited insofar as it is inert to the reaction and dissolves the starting material to a certain degree. The preferable examples include dichloromethane, tetrahydrofuran, ethyl acetate, dimethylformamide etc. Further, this reaction can be conducted in an oxygen atmosphere or an air stream to give good results such as reduction in reaction time, improvement in yield, etc.
Production Method 3 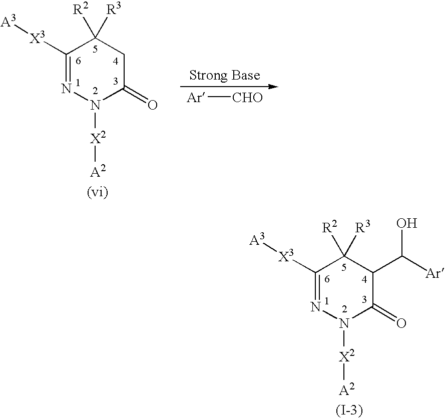
wherein X², X³, A², A³, R² and R³ have the same meanings as defined above; and Ar′ represents an optionally substituted aromatic ring or an optionally substituted heterocyclic group. The compound according to the present invention which is represented by the above formula (1-3) can be produced by introducing a substituent group into the 4-position of the pyridazinone ring in the pyridazinone derivative represented by the formula (vi). A preferable method of introducing the substituent group is, for example, a method of allowing a strong base to act on (vi) to generate an anion at the 4-position and reacting it with an aryl aldehyde. The strong base used varies depending on the starting material, the solvent used etc., and is not particularly limited insofar as it is inert to the reaction. The preferable examples include lithium diisopropylamide, lithium bistrimethylsilylamide etc. This reaction is conducted preferably in the presence of a solvent from viewpoints of operativeness, stirring and temperature control. The solvent varies depending on the starting material, reagents etc., and is not particularly limited insofar as it is inert to the reaction and dissolves the starting material to a certain degree. The preferable examples include tetrahydrofuran, diethyl ether etc. The reaction temperature varies depending on the starting material, the solvent used etc., and is not particularly limited. Usually the temperature is 0° C. or less, preferably −78° C. or less, and under this temperature condition, the yield can be significantly improved.

As the compound according to the present invention which is represented by the above formula (I), the compound represented by the following formula (1-4) wherein Z is N can be produced by converting an α-haloketone derivative (vii) into an α-ketone derivative azide (viii), then condensing (viii) with a hydrazine derivative (ii) to synthesize an α-azide hydrazide derivative (ix), further reducing (ix) to convert it into an α-aminohydrazide derivative (x) and then into a triazinone ring (xi), and introducing a substituent group to the 4-position of the ring, as shown in the reaction scheme:
Production Method 4 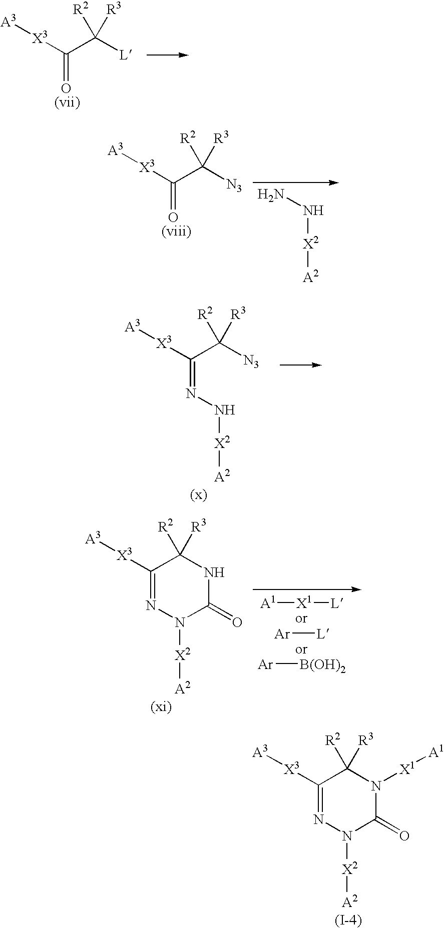
wherein X¹, X², X³, A¹, A², A³, R² and R³ have the same meanings as defined above; L′ represents a halogen atom such as chlorine atom, bromine atom or iodine atom; and Ar represents an optionally substituted aromatic ring or an optionally substituted heterocycle.

The azidating reagent used in the azidation reaction in producing (viii) varies depending on the starting material, the solvent used etc., and is not particularly limited insofar as it is inert to the reaction. The preferable examples include sodium azide, lithium azide etc. The azidation reaction is conducted preferably in the presence of a solvent from viewpoints of operativeness, stirring, safety and the like. The solvent used varies depending on the starting material, reagents etc., and is not particularly limited insofar as it is inert to the reaction and dissolves the starting material to a certain degree. The preferable examples include dimethylformamide, chloroform, dichloromethane, etc. The reaction temperature varies depending on the reagents used, the solvent etc., but usually the reaction is carried out at room temperature or less, preferably under ice-cooling, from viewpoint of safety.

The hydrazine derivative (ii) used in production of (ix) may be a salt, and is not particularly limited insofar as the reaction is not inhibited. From viewpoints of safety and availability, e.g. hydrochloride is preferable. The solvent used varies depending on the starting material, reagents etc., and is not particularly limited insofar as it is inert to the reaction and dissolves the starting material to a certain degree. Preferable examples include ethanol, toluene, chloroform, etc. The reaction temperature varies depending on the reagents used, the solvent etc., but usually the reaction is carried out at room temperature or by heating under reflux. Further, an acid catalyst such as p-toluene sulfonic acid or camphor sulfonic acid can be added as an additive in this reaction to give good results such as reduction in reaction time, improvement in yield, etc.

The conditions for reducing the azide group for production of (x) are not particularly limited insofar the conditions are gentle, and for this reduction, triphenylphosphine is preferably used. The solvent used varies depending on the starting material, reagents etc., and is not particularly limited insofar as it is inert to the reaction and dissolves the starting material to a certain degree. Preferable examples include tetrahydrofuran, chloroform, toluene, etc. The reaction temperature varies depending on the reagents used, the solvent etc., but usually the reaction is carried out at room temperature or by heating under reflux, preferably from 60° C. to 120° C.

For cyclization of the triazinone ring in production of (xi), it is preferable that (x) is reacted with a carbonylation reagent such as triphosgene, 1,1′-carbonyl diimidazole or diethyl carbonate, preferably triphosgene, in the presence of a base such as triethylamine. The solvent used varies depending on the starting material, reagents etc., and is not particularly limited insofar as it is inert to the reaction and dissolves the starting material to a certain degree. Preferable examples include tetrahydrofuran, acetonitrile, etc. The reaction temperature varies depending on the reagents used, the solvent etc., but usually the reaction is carried out under ice-cooling or by heating under reflux.

In the “step of introducing a substituent group to the 4-position of the triazinone derivative (xi)” as the final step for production of the compound (I-4) according to the present invention, a typical method for introducing an aryl group is, for example, Ullman reaction with a halogen aryl derivative. The reaction conditions are not particularly limited, and for example, the reaction is carried out in the presence of copper, copper bromide, copper iodide etc. with a base such as potassium carbonate, sodium carbonate, potassium acetate, sodium acetate, etc. in the system in a solvent under stirring. The solvent used varies depending on the starting material, reagents etc., and is not particularly limited insofar as it is inert to the reaction and dissolves the starting material to a certain degree. Preferable examples include dimethylformamide, dichlorobenzene, nitrobenzene, amyl alcohol, etc. The reaction temperature varies depending on the reagents used, the solvent etc., but usually the reaction can be finished in a short time by heating under reflux. An alternative method of introducing a substituent group to the 5-position of the triazinone derivative (xi) is a method of subjecting (xi) and an arylboronic acid derivative (Ar—B(OH)₂ in the reaction scheme above) to coupling reaction in the presence of a base with a copper compound. Preferably the arylboronic acid derivative used is, for example, an optionally substituted phenylboronic acid derivative or an optionally substituted heterocyclic boronic acid derivative. The base used varies depending on the other reagents used, the solvent used etc., and is not particularly limited insofar as the reaction is not inhibited. Preferable examples include triethylamine, pyridine, tetramethylethylenediamine, etc. Preferably the copper compound used is, for example, copper acetate, di-μ-hydroxo-bis[(N,N,N′,N′-tetramethylethylene diamine) copper (II)] chloride or the like. This reaction is conducted preferably in the presence of a solvent. The solvent used varies depending on the starting material, reagents etc., and is not particularly limited insofar as it is inert to the reaction and dissolves the starting material to a certain degree. Preferable examples include dichloromethane, tetrahydrofuran, ethyl acetate, dimethylformamide, etc. Further, this reaction can be conducted in an oxygen atmosphere or an air stream to give good results such as reduction in reaction time, improvement in yield, etc. A base such as sodium hydride, tert-butoxy potassium etc. can be added to further improve yield.
Production Method 5 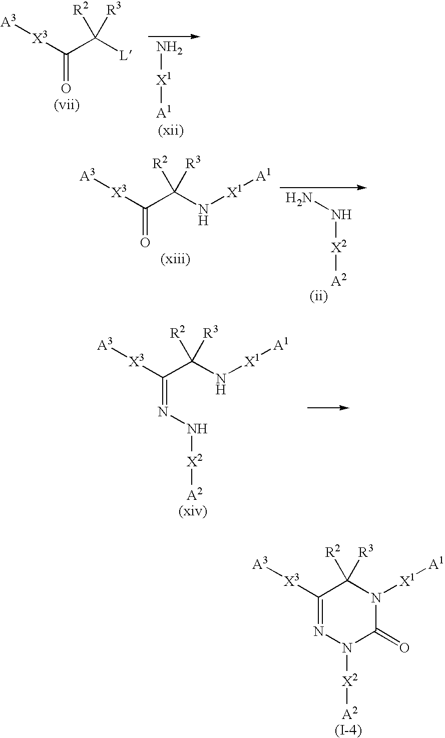
wherein X¹, X², X³, A¹, A², A³, R², R³ and L′ have the same meanings as defined above. The compound represented by the above formula (1-4) according to the present invention can also be produced according to Production Method 5 shown in the above reaction scheme.

That is, an intermediate α-aminoketone derivative (xiii) is produced by condensation reaction of an α-haloketone derivative (vii) with an amine derivative (xii). From viewpoints of operativeness and stirring, this step is conducted preferably in a solvent in the presence of an organic base such as triethylamine, an inorganic base such as potassium carbonate, or an excess of an amine derivative (xii). The solvent used varies depending on the starting material, reagents etc., and is not particularly limited insofar as it is inert to the reaction and dissolves the starting material to a certain degree. Preferable examples include ethanol, acetone, tetrahydrofuran, etc. By adding potassium iodide, sodium iodide etc., good results such as reduction in reaction time, improvement in yield, etc. can be obtained.

An α-aminohydrazide derivative (xiv) is produced by condensation reaction of the α-aminoketone derivative (xiii) with a hydrazine derivative (ii). The substituted hydrazine derivative used may be a salt, and is not particularly limited insofar as the reaction is not inhibited. From viewpoints of stability and availability, it is preferably a hydrochloride. The solvent used varies depending on the starting material, reagents etc., and is not particularly limited insofar as it is inert to the reaction and dissolves the starting material to a certain degree. Preferable examples include ethanol, toluene, chloroform etc. The reaction temperature varies depending on the type of used reagent, solvent, catalyst etc., but usually the reaction is carried out at room temperature or by heating under reflux. An acid catalyst such as p-toluene sulfonic acid or camphor sulfonic acid can be added as an additive to give good results such as reduction in reaction time, improvement in yield, etc.

The “triazinone ring cyclization” which is the final step (i.e. the step from the intermediate (xiv) to (I-4)) for production of the compound (I-4) of the present invention is carried out by reacting (xiv) preferably with a carbonylation reagent such as triphosgene or a carbonylation reagent such as 1,1′-carbonyl diimidazole or diethyl carbonate in the presence of a base such as triethylamine. The reaction is carried out more preferably using triphosgene. The solvent used varies depending on the starting material, reagents etc., and is not particularly limited insofar as it is inert to the reactoin and dissolves the starting material to a certain degree. Preferable examples include tetrahydrofuran, acetonitrile etc. The reaction temperature varies depending on the reagents used, the solvent etc., but usually the reaction is carried out under ice-cooling or by heating under reflux.
Production Method 6 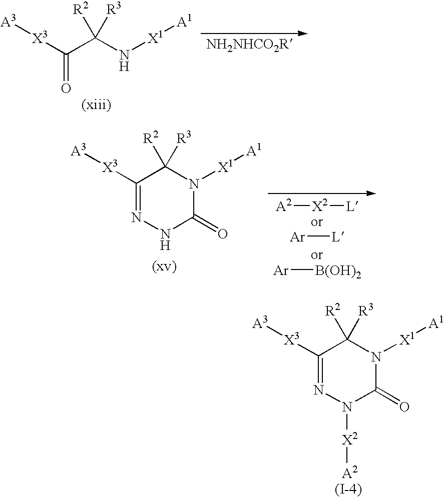
wherein X¹, X², X³, A¹, A², A³, R², R³, L′ and Ar have the same meanings as defined above; and R′ represents a C₁₋₆ alkyl or benzyl group.

The compound according to the present invention which is represented by the formula (I-4) can also be produced by condensation reaction of a hydrazinocarboxylate (NH₂NHCO₂R′ in the reaction scheme) with the aminoketone derivative (xiii) produced in the above-mentioned Production Method 5, to synthesize a triazine derivative (xv), followed by introducing a substituent group to the 2-position of (xv).

The condensation reaction of (xiii) with the hydrazinocarboxylate (NH₂NHCO₂R′ in the reaction scheme) is conducted preferably in the presence of a solvent from viewpoints of operativeness and stirring. The solvent used varies depending on the starting material, reagents etc., and is not particularly limited insofar as it is inert to the reaction and dissolves the starting material to a certain degree. Preferable examples include ethanol, toluene, xylene, etc. The reaction temperature varies depending on the reagents, solvent, catalyst etc., and is not particularly limited, but usually the reaction is carried out at room temperature or by heating under reflux, preferably at 40 to 120° C.

In the “step of introducing a substituent group to the 2-position of the triazinone derivative (xv)” as the final step for production of the compound (I-4) according to the present invention, a typical method for introducing an aryl group is, for example, Ullman reaction with a halogen aryl derivative. The reaction conditions are not particularly limited, and for example, the reaction is carried out in the presence of copper, copper bromide, copper iodide, etc. with a base such as potassium carbonate, sodium carbonate, potassium acetate, sodium acetate, etc. in a solvent under stirring. The solvent used varies depending on the starting material, reagents etc., and is not particularly limited insofar as it is inert to the reaction and dissolves the starting material to a certain degree. Preferable examples include dimethylformamide, dichlorobenzene, nitrobenzene, amyl alcohol, etc. The reaction temperature varies depending on the reagents used, the solvent etc., but usually the reaction can be finished in a short time by heating under reflux. An alternative method of introducing the substituent group to the 2-position of the triazinone derivative (xv) is a method of subjecting (xv) and an arylboronic acid derivative (Ar—B(OH)₂ in the reaction scheme above) to coupling reaction in the presence of a base with a copper compound. Preferable examples of the arylboronic acid derivative used include an optionally substituted phenylboronic acid derivative or an optionally substituted heterocyclic boronic acid derivative. The base used varies depending on the starting material, the solvent used etc., and is not particularly limited insofar as it is inert to the reaction. Preferable examples include triethylamine, pyridine, tetramethylethylenediamine, etc. Preferable examples of the copper compound used include copper acetate, di-μ-hydroxo-bis[(N,N,N′,N′-tetramethylethylenediamine) copper (II)] chloride, and the like. This reaction is conducted preferably in the presence of a solvent, and the solvent used varies depending on the starting material, reagents etc., and is not particularly limited insofar as it is inert to the reaction and dissolves the starting material to a certain degree. Preferable examples include dichloromethane, tetrahydrofuran, ethyl acetate, dimethylformamide, etc. Further, this reaction can be conducted in an oxygen atmosphere or an air stream to give good results such as reduction in reaction time, improvement in yield, etc.

When A¹, A² and/or A³ in the compound (I) in this invention have a substituent group, the substituent group can be easily converted by a known method or its analogous method. For example, (1) when the substituent group is a nitro group and, although there is no particular limitation for the method and for the resulting substance, a method of changing to an amine derivative by a reduction reaction may be exemplified. Although there is usually no particular limitation for the reduction condition, preferred conditions are a method where iron, zinc or tin is used under acidic conditions, a hydrogenation method where palladium, rhodium, ruthenium, platinum or a complex thereof is used as a catalyst. When the amine derivative produced by the said reduction reaction is used, it is possible to further change to an amide compound, a carbamate compound, a sulfonamide compound, a halogen compound, a substituted amine compound etc., easily. (2) When the substituent group is an alkoxy group, an example for changing to a functional group from an alkoxy group is a method to change to an alcohol derivative by means of deprotection. The alcohol derivative which is prepared by the said method may be easily changed to an ester compound by a dehydrating condensation with carboxylic acid derivative or by a reaction with an acid chloride or may be easily changed to an ether compound by a Mitsunobu reaction or by a condensation reaction with a halogen compound. (3) When the substituent is an aldehyde group, various reactions are known for changing to a functional group from an aldehyde group and, although there is no particular limitation for the method therefor and the resulting substance by the change, an example is a method of changing to a carboxylic acid derivative by an oxidation reaction. The carboxylic acid derivative prepared by the said method may be easily changed further to an ester compound, a ketone compound, etc. In addition, starting from the said carboxylic acid derivative, it is possible to easily manufacture an alcohol derivative by a reduction reaction, an amine derivative by a reductive amination reaction, a secondary alcohol compound by an addition reaction with an organic metal reagent and various alkyl derivatives by a Wittig reaction. (4) When the substituent group is a halogen atom, an example for changing to a functional group from a halogen atom as a substituent is a method of changing to a nitrile derivative by a substitution reaction. Besides the above, it is also possible to easily change to various kinds of compounds via, for example, an organolithium compound, an organomagnesium compound, an organotin compound or an organoboronic acid derivative etc.

Described above are typical examples of the method of producing the compound (I) according to the present invention, and the starting compound and various reagents in production of the compound of the invention may be in the form of salts or hydrates, vary depending on the starting material, the solvent used etc., and are not particularly limited so long as it is inert to the reaction. As a matter of course, the solvent used varies depending on the starting material, reagents etc., and is not particularly limited insofar as it is inert to the resaction and dissolves the starting material to a certain degree. When the compound (I) of the present invention is obtained in a free substance, it may be changed to a state of a salt by conventional methods. Further, various isomers (for example, a geometrical isomer, an enantiomer based on an asymmetric carbon, a rotamer, a stereoisomer, a tautomer, and the like) which are obtained for the compound (I) related to the present invention are purified by using usual separation procedures, for example, such as recrystallization, a diastereomer salt method, an enzymolysis method, various chromatographies (for example, thin layer chromatography, column chromatography, gas chromatography, and the like), and can be separated.

The compound according to the present invention which is represented by the above formula (I), a salt thereof or a hydrate of them can be used as they are, or mixed with pharmacologically acceptable carriers known per se, and formed into pharmaceutical preparations by a conventional method. As the preferable preparation forms, tablets, diluted powder, fine granules, granules, coated tablets, capsules, syrup, troche, inhalation preparation, suppositories, injections, ointments, eye ointments, eye drops, nasal preparations, ear drops, cataplasm, lotions etc. may be proposed. In the manufacture of the pharmaceutical preparations, it is possible to use commonly used fillers, binders, disintegrating agent, lubricants, coloring agents, corrigents and, if necessary, stabilizers, emulsifiers, absorption promoters, surfactant, pH adjusting agents, antiseptics, antioxidants, etc. and, after compounding with the ingredients commonly used as materials for the pharmaceutical preparations, it is made into pharmaceutical preparations by a common method.

Examples of the components thereof are animal and plant oil such as soybean oil, beef tallow or synthetic glyceride; hydrocarbon such as liquid paraffin, squalane or solid paraffin; ester oil such as octyldodecyl myristate or isopropyl myristate; higher alcohol such as cetostearyl alcohol or behenyl alcohol; silicone resin; silicone oil; surfactant such as polyoxyethylene fatty acid ester, sorbitan fatty acid ester, glycerol fatty acid ester, polyoxyethylene sorbitan fatty acid ester, polyoxyethylene hydrogenated castor oil or polyoxyethylene-polyoxypropylene block copolymer; water-soluble high-molecular substance such as hydroxyethyl cellulose, polyacrylic acid, carboxyvinyl polymer, polyethylene glycol, polyvinylpyrrolidone or methylcellulose; lower alcohol such as ethanol or isopropanol; polyhydric alcohol such as glycerol, propylene glycol, dipropylene glycol or sorbitol; saccharide such as glucose or sucrose; inorganic powder such as silicic acid anhydride, aluminum magnesium silicate or aluminum silicate; pure water and the like. Applicable examples of a filler are lactose, corn starch, pure sugar, glucose, mannitol, sorbitol, crystalline cellulose, silicon dioxide etc.; those of a binder are polyvinyl alcohol, polyvinyl ether, methyl cellulose, ethyl cellulose, gum arabic, tragacanth, gelatin, shellac, hydroxypropyl methyl cellulose, hydroxypropyl cellulose, polyvinylpyrrolidone, polypropylene glycol-polyoxyethylene block copolymer, meglumine, calcium citrate, dextrin, pectin etc.; those of a disintegrating agent are starch, agar, gelatin powder, crystalline cellulose, calcium carbonate, sodium bicarbonate, calcium citrate, dextrin, pectin, carboxymethyl cellulose calcium etc.; those of a lubricant are magnesium stearate, talc, polyethylene glycol, silica, hydrogenated plant oil etc.; those of a coloring agent are those which are allowed to add to pharmaceuticals; those of a corrigent are cocoa powder, menthol, aromatic powder, peppermint oil, borneol and cinnamon powder; and those of an antioxidant are those which are permitted to be added to pharmaceuticals, such as ascorbic acid, α-tocopherol and the like, are respectively used.

In the manufacture of preparations for oral use, the compound of the present invention or a pharmacologically acceptable salt is mixed with a filler and, if necessary, further with a binder, a disintegrating agent, a lubricant, a coloring agent, a corrigent, etc. and the mixture is made into diluted powder, fine particles, granules, tablets, coated tablets, capsules, etc. by a common method.

In case of tablets and coated tablets, there is of course no problem that such tablets and granules are sugar-coated, gelatin-coated, or appropriately coated upon necessity.

In case of the manufacture of liquid preparations such as syrup, injection preparations and eye drops, a pH adjusting agent, a solubilizer, an isotonizing agent, etc. and, if necessary, a solubilizing aid, a stabilizer, buffer, suspending agent, antioxidant etc. are added, and then made into pharmaceutical preparations by a common method. It can be made as a freeze drying product, and a injections can be dosed in vena, subcutis, and muscle. Preferable examples in a suspending agent include methyl cellulose, polysorbate 80, hydoxyethyl cellulose, gum arabic, tragacanth powder, sodium carboxymethyl cellulose, polyoxyethylene sorbitan monolaurate, and the like; preferable examples in a resolving aid include polyoxyethylene hardened castor oil, polysorbate 80, nicotinic acid amide, polyoxyethylene sorbitan monolaurate, and the like; preferable examples in a stabilizer include sodium sulfite, meta sodium sulfite, ether, and the like; preferable examples in a preservative include methyl p-oxybenzoate, ethyl p-oxybenzoate, sorbic acid, phenol, cresol, chlorocresol and the like.

In case of external use, there is no particular limitation for a method of manufacturing a pharmaceutical preparation, but a common method is used for the manufacture. Thus, with regard to a base material used, various materials which are commonly used for pharmaceuticals, quasi drugs, cosmetics, etc. may be used. Specific examples of the base material used are animal/plant oil, mineral oil, ester oil, waxes, higher alcohols, fatty acids, silicone oil, surfactant, phospholipids, alcohols, polyhydric alcohols, water-soluble high-molecular substances, clay minerals and pure water and, if necessary, it is possible to add pH adjusting agent, antioxidant, chelating agent, antiseptic antifungal, coloring agent, perfume, etc. If necessary, it is further possible to compound other components such as a component having a differentiation-inducing action, blood flow promoter, bactericide, anti-inflammatory agent, cell activator, vitamins, amino acid, moisturizer and keratin solubilizing agent.

The pharmaceutical preparation comprising the compound (I) according to the present invention, a salt thereof or a hydrate of them, as an active ingredient is useful for treatment and prevention in mammalians (e.g., humans, mice, rats, guinea pigs, rabbits, dogs, horses, monkeys etc.), particularly in treatment and prevention in humans. Dose of the pharmaceutical preparation according to the present invention varies depending on degree of symptom, age, sex, body weight, dosage form, type of salt, sensitivity to the pharmaceuticals, specific type of the disease, etc. In humans, the pharmaceutical preparation is given daily in one portion or in divided portions into an adult in a dose of usually about 30 μg to 10 g, preferably 100 μg to 10 g, more preferably 100 μg to 5 g for oral administration, or about 30 μg to 10 g for injection.

In accordance with the present invention, it is possible to provide a novel compound (I) which show an excellent inhibiting action to AMPA receptor and/or kainate receptor and are useful as pharmaceutical agents. Further, a useful production process for producing the compound or its salt and a production intermediate could be provided. According to this process, the compound relating to the present invention can be obtained in high yield, and the highly safe compound can be obtained. The compound (I) of the present invention suppress the neurotoxicity of excitatory neurotransmitters and is able to achieve an excellent neuroprotecting action as a pharmaceutical agent. Accordingly, the compounds of the present invention are useful as therapeutic, preventive and improving agents for various nervous diseases and are useful, for example, as therapeutic and preventive agents for acute neurodegenerative diseases (such as cerebrovascular disorders at acute stage, subarachnoid hemorrhag, head injury, spinal cord injury, neuropathy caused by hypoxia or hypoglycemia etc.), chronic neurodegenerative diseases (such as Alzheimer's disease, Parkinson's disease, Huntington's chorea, amyotrophic lateral sclerosis or spinocerebellar degeneration), epilepsy, hepatic enephalopathy, peripheral neuropathy, Parkinson's syndrome, spasticity, pain, neuralgia, schizophrenia, anxiety, drug abuse, nausea, vomiting, urinary disturbance, visual disturbance due to glaucoma, auditory disturbance due to antibiotics, food poisoning, infectious cerebrospinal meningitis (such as HIV cerebrospinal meningtitis), cerebrovascular dementia, or dementia or nervous symptoms due to meningitis. Further, the compound of the present invention is useful as an agent for treating or preventing demyelinating disorder (such as encephalitis, acute disseminated encephalomyelitis, multiple sclerosis, acute demyelinating polyneuropathy, Guillain Barre syndrome, chronic inflammatory demyelinating polyneuropathy, Marchifava-Bignami disease, central pontine myelinolysis, neuromyelitis optica, Devic syndrome, Balo disease, HIV-myelopathy, HTLV-myelopathy, progressive multifocal leucoencephalopathy and a secondary demyelinating disorder (such as CNS lupus erythematodes, polyarteritis nodosa, Sjogren syndrome, sarcoidosis and isolated cerebral vasulitis)).

### EXAMPLES

Reference Examples, Examples (further pharmacologically acceptable salts thereof, hydrates thereof, and pharmaceutical preparations or pharmaceutical composition comprising them) and Test Example shown below are described merely for illustrative purposes, and the compounds of the invention are not limited to the following examples in any case. The present invention can be carried out to the maximum by those skilled in the art by making various modifications not only to the following examples but also to claims in this specification, and such modifications fall under the claims in this specification.

### Reference Example 1

### 1-(2-Pyridyl)-3-(3-methoxyphenyl)-2-propen-1-one

Potassium tert-butoxide (2.4 g) was added to a solution of 2-acetylpyridine (25 g) and 3-methoxybenzaldehyde (28 g) in tetrahydrofuran (150 ml), followed by stirring for 5 hours. The reaction mixture was partitioned between ethyl acetate and water, and the organic layer was washed with water, dried and concentrated. The residue was purified by silica gel column (ethyl acetate-hexane system), to give the title compound (17.2 g) as a yellow solid.

¹H-NMR(400 MHz, CDCl₃); δ (ppm) 3.87(s, 3H), 6.96-6.99(m, 1H), 7.24-7.26(m, 1H), 7.32-7.34(m, 2H), 7.50(ddd, 1H), 7.88(dt, 1H), 7.91(d, 1H), 8.19(td, 1H), 8.28(d, 1H), 8.75(ddd, 1H).

### Reference Example 2

### 2-(3-Methoxyphenyl)-4-(2-pyridyl)-4-oxobutanenitrile

According to J. Chem. Soc. (1958) 4193, the title compound (16.7 g) was obtained as a brown oil from 1-(2-pyridyl)-3-(3-methoxyphenyl)-2-propen-1-one (17.2 g).

¹H-NMR(400 MHz, CDCl₃); δ (ppm) 3.80 (dd, 1H), 3.82(s, 3H), 4.00(dd, 1H), 4.50(dd, 1H), 6.86(dd, 1H), 6.97(t, 1H), 7.00-7.03(m, 1H), 7.29(t, 1H), 7.50(ddd, 1H), 7.86(td, 1H), 8.07(td, 1H), 8.65(ddd, 1H).

### Reference Example 3

### 2-(3-Methoxyphenyl)-4-(2-pyridyl)-4-oxobutyric acid

According to J. Heterocyclic. Chem., 25, 799 (1988), the title compound (12.3 g) was obtained as a brown solid from 2-(3-methoxyphenyl)-4-(2-pyridyl)-4-oxobutanenitrile (16.7 g).

¹H-NMR(400 MHz, CDCl₃); δ (ppm) 3.52-3.58(m, 1H), 3.77(dd, 1H), 3.79(s, 1H), 8.55(dd, 1H), 6.82(ddd, 1H), 6.85-6.89(m, 1H), 6.94(t, 1H), 6.98(d, 1H), 7.47(ddd, 1H), 7.83(dt, 1H), 8.02(d, 1H), 8.67(ddd, 1H).

### Reference Example 4

### Ethyl 4-(2-methoxyphenyl)-2-(2-pyridyl)-4-oxobutylate

60% sodium hydride (1.5 g) was added to a solution of ethyl 2-pyridylacetate (5.5 g) in dimethylformamide (50 ml) under ice-cooling, followed by stirring. After 1 hour, 2-methoxyphenacyl bromide (7.7 g) was added thereto, and the mixture was stirred for 1 hour under ice-cooling and then stirred overnight at room temperature. The reaction mixture was evaporated, and partitioned between ethyl acetate and water. The organic layer was washed with water, dried and concentrated. The residue was purified by silica gel column (ethyl acetate-hexane system), to give the title compound (6.6 g) as a reddish brown solid.

¹H-NMR(400 MHz, CDCl₃); δ (ppm) 1.20(t, 3H), 3.59(dd, 1H), 3.88(s, 3H), 3.95(dd, 1H), 4.11-4.20(m, 2H), 4.51(dd, 1H), 6.94-7.00(m, 2H), 7.15-7.18(m, 1H), 7.36(dt, 1H), 7.43-7.47(m, 1H), 7.65(td, 1H), 7.73(dd, 1H), 8.54-8.56(m, 1H).

### Reference Example 5

### 4-Phenyl-2,3,4,4a-tetrahydro-5H-(1)benzopyrano[4,3-c]pyridazin-3-one

4-Oxo-4H-2,3-dihydro-1-benzopyran-3-acetic acid (4.00 g) synthesized according to the method described in Example 1 was dissolved in ethanol (60 ml), and hydrazine monohydrate (0.68 g) was added thereto, followed by heating under reflux for 3 hours. After cooling as it was to room temperature, the resulting crystals were separated by filtration, to give the title compound (1.95 g, 49%).

¹H-NMR(400 MHz, DMSO-d₆); δ (ppm) 3.65-3.84(m, 3H), 4.00(dd, 1H), 6.91(dd, 1H), 7.04(ddd, 1H), 7.27-7.41(m, 6H), 7.90(dd, 1H), 11.18(s, 1H).

### Reference Example 6

### 3-Chloro-6-methoxy-5-tributyltin pyridazine

2.5 M butyl lithium (19.4 ml) was added to a solution of diisopropylamine (6.7 ml) in tetrahydrofuran (60 ml) at —40° C. in a nitrogen atmosphere. After stirring for 20 minutes under ice-cooling, a solution of 3-chloro-6-methoxypyridazine (5.76 g) and tributyltin chloride (15.56 g) in tetrahydrofuran (30 ml) was added dropwise thereinto at —72° C. and stirred for 1 hour. Water was added thereto, and the mixture was extracted with ethyl acetate. The organic layer was washed with water, dried and concentrated. Then, the residue was purified by silica gel column chromatography (ethyl acetate/hexane system), to give the title compound (12.77 g) as a pale yellow oil. ¹H-NMR(400 MHz, CDCl₃); δ (ppm) 0.89(t, 9H), 1.10-1.15(m, 6H), 1.27-1.36(m, 6H), 1.48-1.53(m, 6H), 4.06(s, 3H), 7.38(s, 1H).

### Reference Example 7

### 3-Chloro-6-methoxy-5-phenylpyridazine

3-Chloro-6-methoxy-5-tributyltin pyridazine (3.20 g), bromobenzene (11.57 g), tetrakis(triphenylphosphine)palladium (428 mg) and copper (I) iodide (70 mg) were added to xylene (130 ml), followed by stirring at 120° C. for 2 hours in a nitrogen atmosphere. The reaction mixture was purified by silica gel column chromatography (ethyl acetate/hexane system), to give the title compound (1.10 g) as a colorless oil.

¹H-NMR(400 MHz, CDCl₃); δ (ppm) 4.16(s, 3H), 7.40(s, 1H), 7.47-7.50(m, 3H), 7.60-7.63(m, 2H).

### Reference Example 8

### 6-Chloro-4-phenyl-3(2H)-pyridazinone

A reaction mixture of 3-chloro-6-methoxy-5-phenylpyridazine (267 mg) and conc. hydrochloric acid (2 ml) was heated under reflux for 2 hours. After concentrating, the residue was purified by silica gel column chromatography (ethyl acetate), to give the title compound (159 mg) as a colorless solid.

¹H-NMR(400 MHz, CDCl₃); δ (ppm) 7.38(s, 1H), 7.47-7.49(m, 3H), 7.81-7.84(m, 2H), 11.34(brs, 1H).

### Reference Example 9

### 6-Chloro-2-(2-cyanophenyl)-4-phenyl-3(2H)-pyridazinone

A suspension of 6-chloro-4-phenyl-3(2H)-pyridazinone (80 mg), 2-(2-cyanophenyl)-1,3,2-dioxaborinate (144 mg), copper (II) acetate (35 mg), triethylamine (107 μl) and pyridine (62 μl) in methylene chloride (5 ml) was stirred for 4 days in an oxygen atmosphere. The reaction mixture was partitioned between aqueous ammonia and ethyl acetate, and the organic layer was washed with water, dried and concentrated. Then, the residue was purified by silica gel column chromatography (ethyl acetate/hexane system) to give the title compound (83 mg) as a colorless solid.

¹H-NMR(400 MHz, CDCl₃); δ (ppm) 7.43(s, 1H), 7.46-7.50(m, 3H), 7.56(dt, 1H), 7.68(ddd, 1H), 7.76(ddd, 1H), 7.81-7.84(m, 3H).

### Reference Example 10

### 3-Methoxy-4-phenyl-6-(2-pyrimidinyl)pyridazine

2-Tributylstannylpyrimidine (2.10 g) prepared according to Tetrahydron 50, 275-284 (1994), 3-chloro-6-methoxy-5-phenylpyridazine (800 mg) and tetrakis(triphenylphosphine)palladium (208 mg) were added to xylene (10 ml), followed by stirring at 120° C. for 2 hours in a nitrogen atmosphere. The reaction mixture was purified by NH-silica gel column chromatography (ethyl acetate/hexane system), to give the title compound (403 mg) as a brown solid.

¹H-NMR(400 MHz, CDCl₃); δ (ppm) 4.28(s, 3H), 7.37(t, 1H), 7.46-7.53(m, 3H), 7.73-7.76(m, 2H), 8.52(s, 1H), 8.94(d, 2H).

### Reference Example 11

### 4-Phenyl-6-(2-pyrimidinyl)-3(2H)-pyridazinone

A solution of 3-methoxy-4-phenyl-6-(2-pyrimidinyl) pyridazine (1.07 g) in 5 N hydrochloric acid (15 ml) was heated under reflux for 2 hours. After neutralizing with 5 N aqueous sodium hydroxide solution, the resulting precipitates were collected by filtration and washed with ethyl acetate, to give the title compound (609 mg) as a colorless solid.

¹H-NMR(400 MHz, CDCl₃); δ (ppm) 7.38(t, 1H), 7.47-7.53(m, 3H), 7.95-7.98(m, 2H), 8.60(s, 1H), 8.95(d, 2H).

### Reference Example 12

### 2-(Azide acetyl)pyridine

Acetyl pyridine (2.46 g) was dissolved in acetic acid (4 ml), and bromine (1.1 ml) was added gradually dropwise thereto under heating at 70° C. After cooling the reaction solution as it was to room temperature, the resulting crystals were collected by filtration. An aqueous sodium bicarbonate solution was added to the crude crystals (4.8 g), followed by extracting with ethyl acetate. The organic layer was washed with water and dried over sodium sulfate anhydride. After the drying agent was filtered off, the filtrate was evaporated. The residue (4.06 g) was dissolved in dimethylformamide (50 ml), sodium azide (1.5 g) was added thereto, and the mixture was stirred at room temperature for 2 hours. Water was added thereto, followed by extracting with ethyl acetate. The organic layer was washed with water and dried over sodium sulfate anhydride. After the drying agent was filtered off, the filtrate was evaporated, to give the title compound as a brown oil (2.74 g, 83%).

¹H-NMR (400 MHz, CDCl₃); δ (ppm) 4.87 (s, 2H), 7.51-7.55 (m, 1H), 7.87-7.91 (m, 1H), 8.09 (d, J=8.0 Hz, 1H), 8.66 (dt, J=4.8 Hz, 0.8 Hz, 1H).

### Reference Example 13

### 2-Pyridyl-aminomethyl-2′-bromophenylhyrazone

2-(Azide acetyl) pyridine (2.7 g) was dissolved in ethanol (50 ml) and 2-bromophenylhydrazine (3.4 g) was added thereto, followed by stirring overnight at room temperature. The reaction mixture was evaporated, and the residue was dissolved in tetrahydrofuran (40 ml). Triphenylphosphine (4.94 g) was added thereto, followed by stirring at room temperature for 3 hours. Water (1 ml) was added thereto, and the mixture was stirred for 1 hour and then heated overnight at 80° C. After cooling to room temperature, the mixture was evaporated. The residue was purified by silica gel column chromatography (hexane-ethyl acetate system), to give the title compound (2.69 g, 53%) as a brown oil.

¹H-NMR (400 MHz, CDCl₃); δ (ppm) 1.84 (brs, 2H), 4.41 (s, 2H), 6.74 (td, J=8.4 Hz, 1.2 Hz, 1H), 7.16-7.17 (m, 1H), 7.25-7.30 (m, 1H), 7.45 (dd, J=8.0 Hz, 1.2 Hz, 1H), 7.62 (dd, J=8.4 Hz, 1.6 Hz, 1H), 7.67 (td, J=8.0 Hz, 0.8 Hz, 1H), 8.14 (d, J=8.0 Hz, 1H), 8.50-8.51 (m, 1H), 11.39 (brs, 1H).

### Reference Example 14

### 2-(2-Bromophenyl)-6-(2-pyridyl)-4,5-dihydro-1,2,4-triazin-3-(2H)-one

2-Pyridyl-aminomethyl-2′-bromophenylhydrazone (2.69 g) was dissolved in tetrahydrofuran anhydride (100 ml), followed by adding triphosgene (1.31 g) and triethylamine (2.7 ml) under ice-cooling. While raising the temperature of the mixture gradually from 0° C. to room temperature, the mixture was stirred overnight. The insoluble matters were filtered off, and the filtrate was evaporated. The residue was purified by NH-silica gel column chromatography (hexane-ethyl acetate system), to give the title compound (1.00 g, 31%) as a brown powder.

¹H-NMR (400 MHz, CDCl₃); δ (ppm) 4.79 (s, 2H), 5.50 (s, 1H), 7.23-7.30 (m, 2H), 7.39-7.48 (m, 1H), 7.53 (dd, J=7.8 Hz, 1.8 Hz, 1H), 7.67-7.72 (m, 2H), 8.04 (d, J=8.0 Hz, 1H), 8.57 (ddd, J=4.8 Hz, 1.8 Hz, 1.0 Hz, 1H).

ESI-Mass; 331 [M⁺+H]

### Example 1

### 2-(2-Bromophenyl)-4-(3-methoxyphenyl)-6-(2-pyridyl)-4,5-dihydro-3(2H)-pyridazinone

According to Indian J. Chem., Sect. B30 (1991) 6, 589, the title compound (1.1 g) was obtained as a brown solid from 2-(3-methoxyphenyl)-4-(2-pyridyl)-4-oxobutyric acid (3 g) and 2-bromophenylhydrazine (2 g).

¹H-NMR(400 MHz, CDCl₃); δ (ppm) 3.44-3.86(m, 2H), 3.73(s, 3H), 3.94-4.15(m, 1H), 6.81(dd, 1H), 6.97-7.01(m, 2H), 7.21-7.29(m, 3H), 7.37-7.41(m, 2H), 7.65-7.69(m, 2H), 8.05(d, 1H), 8.61-8.63(m, 1H).

### Example 2

### 2-(2-Bromophenyl)-4-(3-hydroxyphenyl)-6-(2-pyridyl)-4,5-dihydro-3(2H)-pyridazinone

A solution of 1 M boron tribromide in methylene chloride (7 ml) was added to a solution of 2-(2-bromophenyl)-4-(3-methoxyphenyl)-6-(2-pyridyl)-4,5-dihydro-3(2H)-pyridazinone (1.1 g) in dichloromethane (20 ml) under ice-cooling, followed by stirring for 1 hour. Ice was added thereto, and the reaction mixture was partitioned between aqueous ammonia and ethyl acetate. The organic layer was dried and concentrated, and then the product suspended in ether was collected by filtration, to give the title compound (0.8 g) as a pale brown solid.

¹H-NMR(400 MHz, CDCl₃); δ (ppm) 4.00(brs, 2H), 4.12(brs, 1H), 6.68(dd,1H), 6.84-6.89(m,2H), 7.13(t,1H), 7.23-7.31 (m,2H), 7.36-7.46(m,2H), 7.65-7.71(m,2H), 8.04(d,1H), 8.60-8.62(m,1H).

### Example 3

### 2-(2-Bromophenyl)-4-[3-(2-hydroxyethoxy)phenyl]-6-(2-pyridyl)-3(2H)-pyridazinone

60% sodium hydride (40 mg) was added to a solution of 2-(2-bromophenyl)-4-(3-hydroxyphenyl)-6-(2-pyridyl)-4,5-dihydro-3(2H)-pyridazinone (173 mg) in dimethylformamide (5 ml) under ice-cooling on ice. After stirring for 30 minutes, (2-bromoethoxy)-tert-butyldimethylsilane (300 mg) was added thereto and stirred overnight at room temperature. Ethyl acetate was added thereto, and the mixture was washed with water, dried, concentrated, and then purified by silica gel column (ethyl acetate-hexane system). The resulting brown oil was dissolved in tetrahydrofuran (5 ml), and 5 N hydrochloric acid (1 ml) was added thereto. After stirring for 10 minutes, the mixture was neutralized with 5 N sodium hydroxide and extracted with ethyl acetate. The organic layer was washed with water, dried and concentrated. The residue was purified by silica gel column (ethyl acetate-hexane system), to give the title compound (111 mg) a pale red amorphous.

¹H-NMR(400 MHz, CDCl₃); δ (ppm) 3.94-3.99(m, 2H), 4.16(t, 2H), 7.02(dd, 1H), 7.32-7.41(m, 3H), 7.49-7.58(m, 3H), 7.68-7.79(m, 3H), 8.14(dt, 1H), 8.67-8.68(m, 2H).

### Example 4

### 2-(2-Cyanophenyl)-4-[3-(2-hydroxyethoxy)phenyl]-6-(2-pyridyl)-3(2H)-pyridazinone

Copper (I) cyanide (20 mg) was added to a solution of 2-(2-bromophenyl)-4-[3-(2-hydroxyethoxy)phenyl]-6-(2-pyridyl)-3(2H)-pyridazinone (70 mg) in dimethylformamide (3 ml), followed by stirring at 120° C. for 1 hour. The reaction solution was partitioned between ethyl acetate and ammonia water, and the organic layer was washed with water, dried and concentrated. Then, the residue was purified by silica gel column (ethyl acetate-hexane system), to give the title compound (50 mg) as a colorless solid.

¹H-NMR(400 MHz, CDCl₃); δ (ppm) 3.96-4.00(m, 2H), 4.16(t, 2H), 7.04(ddd, 1H), 7.35(ddd, 1H), 7.40(t, 1H), 7.52-7.61(m, 3H), 7.74-7.89(m, 4H), 8.22(dt, 1H), 8.64(s, 1H), 8.67-8.69(m, 1H).

### Example 5

### 2-(2-Bromophenyl)-6-(2-methoxyphenyl)-4-(2-pyridyl)-3(2H)-pyridazinone.

2-Bromophenylhydrazine (681 mg) was added to a solution of ethyl 4-(2-methoxyphenyl)-2-(2-pyridyl)-4-oxobutyrate (1.14 g) in ethanol (16 ml), followed by stirring at 80° C. for 3 hours. The reaction mixture was concentrated, and then nitrobenzene (20 mg) was added thereto and stirred overnight at 180° C. The reaction mixture was partitioned between ethyl acetate and water, and the organic layer was washed with water, dried and concentrated. The residue was purified by silica gel column (ethyl acetate-hexane system), to give the title compound (294 mg) as a brown solid.

¹H-NMR(400 MHz, CDCl₃); δ (ppm) 3.91(s, 3H), 6.99-7.05(m, 2H), 7.31-7.42(m, 3H), 7.48(td, 1H), 7.55(dd, 1H), 7.62(dd, 1H), 7.75-7.80(m, 2H), 8.70-8.72(m, 1H), 8.74(dt, 1H), 8.78(s, 1H).

### Example 6

### 2-(2-Cyanophenyl)-4-phenyl-2,3,4,4a-tetrahydro-5H-(1)benzopyrano[4,3-c]pyridazin-3-one

4-Phenyl-2,3,4,4a-tetrahydro-5H-(1)benzopyrano[4,3-c]pyridazin-3-one (974 mg) was dissolved in methylene chloride (20 ml), and 2-(2-cyanophenyl)-1,3,2-dioxaborinate (1.96 g), copper acetate (1.27 g) and triethylamine (1.06 g) were added thereto, followed by stirring overnight at room temperature. The reaction solution was diluted with ethyl acetate, washed with aqueous ammonia, 1 N hydrochloric acid and brine, and dried over anhydrous magnessium sulfate. After the drying agent was filtered off, the filtrate was evaporated. The residue was purified by silica gel column (hexane-ethyl acetate system). The resulting crude crystals were recrystallized from ethyl acetate-hexane, to give the title compound (140 mg, 11%).

¹H-NMR(400 MHz, CDCl₃); δ (ppm) 3.66-3.79(m, 2H), 3.93-4.01(m, 1H), 4.04-4.13(m, 1H), 6.89-6.94(m, 1H), 6.99-7.05(m, 1H), 7.29-7.40(m, 4H), 7.41-7.48(m, 3H), 7.61-7.72(m, 2H), 7.72-7.77(m, 1H), 8.03(dd, 1H).

### Example 7

### 2-(2-Cyanophenyl)-4-phenyl-2,3-dihydro-5H-(1)benzopyrano[4,3-c]pyridazin-3-one

2-(2-Cyanophenyl)-4-phenyl-2,3,4,4a-tetrahydro-5H-(1)benzopyrano[4,3-c]pyridazin-3-one (91 mg) was dissolved in acetic acid (4 ml) at 60° C., and bromine (42 mg) was added thereto, and the mixture was stirred at 70° C. for 30 minutes. The reaction solution was diluted with diethyl ether, washed with water and an aqueous saturated sodium bicarbonate solution and dried over anhydrous magnesium sulfate. The drying agent was filtered off, and the filtrate was evaporated and purified by NH silica gel column (hexane-ethyl acetate system), to give the title compound (14 mg, 15%).

¹H-NMR(400 MHz, CDCl₃); δ (ppm) 5.06(s, 2H), 6.98-7.02(m, 1H), 7.07-7.13(m, 1H), 7.33-7.38(m, 1H), 7.38-7.43(m, 2H), 7.46-7.58(m,4H), 7.71-7.80(m,2H), 7.82-7.87(m,1H), 8.03-8.08(m,1H).

### Example 8

### 2-(2-Iodophenyl)-4-(3-pyridyl)-2,3,4,4a-tetrahydro-5H-(1)benzopyrano[4,3-c]pyridazin-3-one

The title compound was synthesized according to the method described in Example 6.

¹H-NMR(400 MHz, CDCl₃); δ (ppm) 3.65-3.83(m, 2H), 3.95-4.10(m, 2H), 6.93(d, 1H), 6.97-7.05(m, 1H), 7.10-7.17(m, 1H), 7.29-7.37(m, 1H), 7.37-7.44(m, 1H), 7.44-7.56(m, 2H), 7.64-7.77(m, 1H), 7.90-8.03(m, 2H), 8.54(d, 1H), 8.64(dd, 1H).

### Example 9

### 2-(2-Cyanophenyl)-4-(3-pyridyl)-2,3-dihydro-5H-(1)benzopyrano[4,3-c]pyridazin-3-one

2-(2-Iodophenyl)-4-(3-pyridyl)-2,3,4,4a-tetrahydro-5H-(1)benzopyrano[4,3-c]pyridazin-3-one (75 mg) was dissolved in 1-methyl-2-pyrrolidone (2 ml), and zinc cyanide (55 mg) and tetrakis(triphenylphosphine) palladium (5 mg) were added thereto, followed by stirring at 100° C. for 1 hour. The reaction solution was diluted with ethyl acetate, washed with an aqueous saturated sodium bicarbonate solution and dried over anhydrous magnesium sulfate. After the drying agent was filtered off, the filtrate was evaporated. The residue was purified by silica gel column (hexane-ethyl acetate system), to give the title compound (34 mg, 57%).

¹H-NMR(400 MHz, DMSO-d₆); δ (ppm) 5.21(s, 2H), 7.10(d, 1H), 7.12-7.20(m, 1H), 7.42-7.48(m, 1H), 7.56-7.61(m, 1H), 7.70-7.77(m,1H), 7.88-8.00(m,4H), 8.07-8.12(m,1H), 8.64-8.75(m,2H).

### Example 10

### 4-(4-Methoxybenzyl)-6-phenyl-2-(2-tolyl)-3(2H)-pyridazinone

6-Phenyl-2-(2-tolyl)-2,3,4,5-tetrahydropyridazin-3(2H)-one (0.54 g) synthesized from 3-benzoylpropionic acid (CAS No. 2051-95-8) and 2-tolylhydrazine hydrochloride (CAS No. 635-26-7) was dissolved in tetrahydrofuran (20 ml). After cooling to −78° C., 1.5 M lithium diisopropylamide (1.2 ml) was gradually added thereto. Then, a solution of 4-anisaldehyde (0.27 g) in tetrahydrofuran (10 ml) was gradually added thereto, followed by stirring overnight so that its temperature was increased to room temperature. Ethyl acetate was added to the reaction solution, and the mixture was washed with brine and water. Then, the solvent was evaporated and the residue was purified by silica gel column (hexane-ethyl acetate system), to give 0.10 g of the title compound.

¹H-NMR(400 MHz, CDCl₃); δ (ppm) 2.19(s, 3H), 3.83(s, 3H), 3.95(s, 2H), 6.88-6.94(m, 2H), 7.24-7.28(m, 2H), 7.29-7.36(m, 4H), 7.37-7.40(m, 2H), 7.45-7.48(m, 1H), 7.66-7.70(m, 2H), 7.82-7.85(m, 1H).

### Example 11

### 2,6-Diphenyl-4-(a-hydroxy-2-picolyl)-4,5-dihydro-3(2H)-pyridazinone

2,6-Diphenyl-2,3,4,5-tetrahydropyridazin-3(2H)-one (0.10 g) synthesized from 3-benzoylpropionic acid (CAS No. 2051-95-8) and phenylhydrazine hydrochloride (CAS No. 100-63-0), and 2-pyridine carboxyaldehyde (0.05 g), were dissolved in tetrahydrofuran (10 ml). After cooling to −78° C., 1.5 M lithium diisopropylamide (0.2 ml) was gradually added thereto. The mixture was reacted for 1 hour, and then 2-pyridine carboxyaldehyde (0.05 g) and 1.5 M lithium diisopropylamide (0.2 ml) were further added thereto. After stirring for 2 hours, the temperature was gradually increased to room temperature. Ethyl acetate was added to the reaction solution, and the mixture was washed with brine and water. Then, the solvent was evaporated, and the residue was purified by silica gel column (hexane-ethyl acetate system), to give the title compound (23 mg).

¹H-NMR(400 MHz, CDCl₃); δ (ppm) 2.75(dd, 1H), 3.13(dd, 1H), 3.23(ddd, 1H), 4.25(brs, 1H), 5.68(brs, 1H), 7.24-7.31(m, 2H), 7.33-7.39(m, 3H), 7.40-7.46(m, 2H), 7.51(dd, 1H), 7.59-7.63(m, 2H), 7.66-7.71(m, 2H), 7.76(dt, 1H), 8.55-8.58(m, 1H).

### Example 12

### 2-(2-Cyanophenyl)-4-(4-morpholinoethylaminocarbonyl)-6-phenyl-3(2H)-pyridazinone

Ethyl 2-ethoxycarbonyl-4-phenyl-4-oxo-butyrate was prepared from 2-bromoacetophenone and diethyl malonate and then reacted with hydrazine monohydrate to synthesize 6-phenyl-4-ethoxycarbonyl-4,5-dihydro-3(2H)-pyridazinone. Then, it was reacted with bromine in acetic acid to give 6-phenyl-4-ethoxycarbonyl-3(2H)-pyridazinone. 6-Phenyl-4-ethoxycarbonyl-3(2H)-pyridazinone (2.00 g) was dissolved in dichloromethane (50 ml), then 4-(2-aminoethyl)morpholine (1.60 g) was added thereto. After heating under reflux for 2 days, it was purified by silica gel column (dichloromethane-methanol system) and converted in a usual manner with methanol/hydrochloric acid, to give 4-(4-morpholinoethylaminocarbonyl)-6-phenyl-3(2H)-pyridazinone hydrochloride (1.83 g). 4-(4-Morpholinoethylaminocarbonyl)-6-phenyl-3(2H)-pyridazinone hydrochloride (0.36 g) and 2-bromobenzonitrile (0.50 g) were dissolved in 1,2-dichlorobenzene (15 ml), and copper (0.2 g) and potassium acetate (1.0 g) were added thereto, followed by stirring at 190° C. for 1 hour. Ethyl acetate was added to the reaction solution, and the mixture was washed with water. The solvent was evaporated and the residue was purified by silica gel column (hexane-ethyl acetate system), to give 13 mg of the title compound.

¹H-NMR(400 MHz, CDCl₃); δ (ppm) 2.48-2.66(m, 6H), 3.58-3.76(m, 6H), 7.45-7.51(m, 3H), 7.62(dt, 1H), 7.70(dd, 1H), 7.80(dt, 1H), 7.86-7.94(m, 3H), 8.84(s, 1H), 9.58(brs, 1H).

### Example 13

### 2-(2-Cyanophenyl)-6-(2-pyridyl)-4,5-dihydro-2H-pyridazino[4,5-b]benzofuran-3-one

Copper (I) cyanide (85 mg) was added to a solution of 2-(2-bromophenyl)-4-(2-hydroxyphenyl)-6-(2-pyridyl)-4,5-dihydro-3(2H)-pyridazinone (200 mg) in dimethylformamide (6 ml), followed by stirring at 120° C. for 50 minutes. The mixture was partitioned between ethyl acetate and aqueous ammonia, and the organic layer was washed with water, dried and concentrated. Then, the residue was purified by silica gel column (ethyl acetate-hexane system), to give the title compound (31 mg) as a pale brown solid.

¹H-NMR(400 MHz, CDCl₃); δ (ppm) 7.46(ddd, 1H), 7.54(td, 1H), 7.60(td, 1H), 7.65(ddd, 1H), 7.77-7.85(m, 3H), 7.88-7.93(m, 2H), 8.26(td, 1H), 8.33-8.35(m, 1H), 8.88-8.90(m, 1H).

The title compounds of Examples 14 to 28 were synthesized according to the method described in the above-mentioned Example 1.

### Example 14

### 2-(2-Bromophenyl)-4-(2-methoxyphenyl)-6-(2-pyridyl)-4,5-dihydro-3(2H)-pyridazinone

¹H-NMR(400 MHz, CDCl₃); δ (ppm) 3.47(brs, 1H), 3.80(brs, 1H), 3.84(s, 3H), 4.29(brs, 1H), 6.90-6.95(m, 2H), 6.96-7.29(m, 3H), 7.43(td, 1H), 7.51-7.54(m, 1H), 7.65-7.71(m, 2H), 8.07(dt, 1H), 8.58(d, 1H).

### Example 15

### 2-(2-Bromophenyl)-4-(4-methoxyphenyl)-6-(2-pyridyl)-4,5-dihydro-3(2H)-pyridazinone

¹H-NMR(400 MHz, CDCl₃); δ (ppm) 3.78(s, 3H), 3.79(brs, 2H), 4.04(brs, 1H), 6.86(d, 2H), 7.24-7.34(m, 4H), 7.37-7.44(m, 2H), 7.67-7.71(m, 2H), 8.04-8.08(m, 1H), 8.62-8.64(m, 1H).

### Example 16

### 2-(2-Bromophenyl)-4-(3-bromo-6-methoxyphenyl)-6-(2-pyridyl)-4,5-dihydro-3(2H)-pyridazinone

¹H-NMR(400 MHz, CDCl₃); δ (ppm) 3.36-3.46(m, 1H), 3.82(s, 3H), 3.82-3.90(m, 1H), 4.14-4.26(m, 1H), 6.78(d, 1H), 7.25-7.29(m, 2H), 7.35-7.52(m, 4H), 7.66-7.71(m, 2H), 8.07(d, 2H), 8.58(d, 1H).

### Example 17

### 2-(2-Iodophenyl)-4-(2-methoxyphenyl)-6-(2-pyridyl)-4,5-dihydro-3 (2H)-pyridazinone

¹H-NMR(400 MHz, CDCl₃); δ (ppm) 3.85(s, 3H), 3.86(brs, 1H), 4.30(brs, 1H), 6.90-6.97(m, 2H), 7.08-7.13(m, 1H), 7.24-7.79(m, 3H), 7.44-7.51(m, 2H), 7.66-7.71(m, 1H), 7.95(dd, 1H), 8.09(d, 1), 8.56-8.59(m, 1H).

### Example 18

### 4-(2-Methoxyphenyl)-2-phenyl-6-(2-pyridyl)-4,5-dihydro-3(2H)-pyridazinone

¹H-NMR(400 MHz, CDCl₃); δ (ppm) 3.47(dd, 1H), 3.75(dd, 1H), 3.85(s, 3H), 4.28(dd, 1H), 6.91-6.95(m, 2H), 7.21(dd, 1H), 7.24-7.30(m, 3H), 7.41-7.45(m, 2H), 7.65-7.73(m, 3H), 8.15(dt, 1H), 8.56-8.58(m, 1H).

### Example 19

### 2-(2-Bromophenyl)-4-phenyl-6-(2-pyridyl)-4,5-dihydro-3(2H)-pyridazinone

¹H-NMR(400 MHz, CDCl₃); δ (ppm) 3.50-3.70(m, 1H), 4.00-4.20(m, 2H), 7.24-7.35(m, 5H), 7.39-7.44(m, 4H), 7.66-7.72(m, 2H), 8.06(d, 1H), 8.62-8.64(m, 1H).

### Example 20

### 2-(2-Bromophenyl)-4-phenyl-6-(3-pyridyl)-4,5-dihydro-3(2H)-pyridazinone

¹H-NMR(400 MHz, CDCl₃); δ (ppm) 3.40-3.49(m, 2H), 4.01-4.14(m, 1H), 7.23-7,47(m, 9H), 7.67(d, 1H), 8.07(d, 1H), 8.62(dd, 1H), 8.95(s, 1H).

### Example 21

### 4,6-Diphenyl-2-(2-pyridyl)-4,5-dihydro-3(2H)-pyridazinone

¹H-NMR(400 MHz, CDCl₃); δ (ppm) 3.42(dd, 2H), 4.05(dd, 1H), 7.22-7.40(m, 9H), 7.56(dt, 1H), 7.76-7.80(m, 1H), 8.59-8.61(m, 1H).

### Example 22

### 4-(2-Methoxyphenyl)-2-(2-pyridyl)-6-(2-pyridyl)-4,5-dihydro-3(2H)-pyridazinone

¹H-NMR(400 MHz, CDCl₃); δ (ppm) 3.51(dd, 1H), 3.79(dd, 1H), 3.81(s, 3H), 4.32(dd, 1H), 6.88-6.95(m, 2H), 7.21-7.28(m, 4H), 7.60-7.69(m, 2H), 7.75-7.79(m, 1H), 8.15(dt, 1H), 8.54-8.55(m, 1H), 8.61-8.62(m, 1H).

### Example 23

### 4-(2-Cyanophenyl)-2-phenyl-6-(2-pyridyl)-3(2H)-pyridazinone

¹H-NMR(400 MHz, CDCl₃); δ (ppm) 7.35(ddd, 1H), 7.42-7.46(m, 1H), 7.51-7.57(m, 3H), 7.70(td, 1H), 7.75-7.83(m, 5H), 8.21(dt, 1H), 8.65(s, 1H), 8.65-8.67(m, 1H).

### Example 24

### 2-(2-Bromophenyl)-4-(2-methoxyphenyl)-6-(3-pyridyl)-4,5-dihydro-3(2H)-pyridazinone

¹H-NMR(400 MHz, CDCl₃); δ (ppm) 3.35(dd, 1H), 3.45(dd, 1H), 3.87(s, 3H), 4.33(brs, 1H), 6.93-6.99(m, 2H), 7.23-7.32(m, 4H), 7.44(td, 1H), 7.70(dd, 1H), 8.05-8.10(m, 1H), 8.60(dd, 1H), 8.89-8.93(m, 1H).

### Example 25

### 4-(2-Bromophenyl)-2-phenyl-6-(2-pyridyl)-4,5-dihydro-3(2H)-pyridazinone

¹H-NMR(400 MHz, CDCl₃); δ (ppm) 3.41(dd, 1H), 3.87(dd, 1H), 4.49(dd, 1H), 7.13-7.17(m, 1H), 7.25-7.34(m, 4H), 7.42-7.47(m, 2H), 7.60-7.74(m, 4H), 8.15(dt, 1H), 8.57-8.59(m, 1H).

### Example 26

### 2-(2-Methoxyphenyl)-4-phenyl-6-(2-pyridyl)-4,5-dihydro-3(2H)-pyridazinone

¹H-NMR(400 MHz, CDCl₃); δ (ppm) 3.58(dd, 1H), 3.77(s, 3H), 3.78-3.87(m, 1H), 4.08(t, 1H), 7.00(d, 1H), 7.03(td, 1H), 7.23-7.43(m, 8H), 7.64(ddd, 1H), 8.03(dt, 1H), 8.58-8.60(m, 1H).

### Example 27

### 4-Phenyl-2-(2-nitrophenyl)-6-(2-pyridyl)-4,5-dihydro-3(2H)-pyridazinone

¹H-NMR(400 MHz, CDCl₃); δ (ppm) 3.70(dd, 1H), 3.78(dd, 1H), 4.06(dd, 1H), 7.26-7.39(m, 6H), 7.46-7.52(m, 1H), 7.67-7.74(m, 3H), 7.97-8.02(m, 2H), 8.61-8.84(m, 1H).

### Example 28

### 2-(2-Fluorophenyl)-4-phenyl-6-(2-pyridyl)-4,5-dihydro-3(2H)-pyridazinone

¹H-NMR(400 MHz, CDCl₃); δ (ppm) 3.64(dd, 1H), 3.83(dd, 1H), 4.10(dd, 1H), 7.15-7.40(m, 9H), 7.46(dd, 1H), 7.70(ddd, 1H), 8.07(dt, 1H), 8.61-8.63(m, 1H).

The title compounds of Examples 29 to 34 were synthesized according to the method described in the above-mentioned Example 2.

### Example 29

### 2-(2-Bromophenyl)-4-(2-hydroxyphenyl)-6-(2-pyridyl)-4,5-dihydro-3(2H)-pyridazinone

¹H-NMR(400 MHz, CDCl₃); δ (ppm) 3.47(brs, 1H), 4.25(brs, 1H), 4.43(brs, 1H), 6.81(td, 1H), 6.97(d, 1H), 7.10(d, 1H), 7.18(td, 1H), 7.25-7.44(m, 4H), 7.68(brs, 1H), 7.74(td, 1H), 8.10(dt, 1H), 8.72(d, 1H).

### Example 30

### 2-(2-Bromophenyl)-4-(4-hydroxyphenyl)-6-(2-pyridyl)-4,5-dihydro-3(2H)-pyridazinone

¹H-NMR(400 MHz, CDCl₃); δ (ppm) 3.60(brs, 2H), 4.02(brs, 1H), 5.20(s, 1H), 6.74(d, 2H), 7.23-7.32(m, 4H), 7.38-7.44(m, 2H), 7.67-7.72(m, 2H), 8.06(d, 1H), 8.62-8.64(m, 1H).

### Example 31

### 2-(2-Bromophenyl)-6-(2-hydroxyphenyl)-4-(2-pyridyl)-3(2H)-pyridazinone

¹H-NMR(400 MHz, CDCl₃); δ (ppm) 7.01-7.05(m, 2H), 7.34-7.43(m, 3H), 7.50-7.56(m, 2H), 7.89(d, 1H), 8.76-8.79(m, 2H), 9.08(s, 1H), 10.50(s, 1H).

### Example 32

### 4-(2-Hydroxyphenyl)-2-phenyl-6-(2-pyridyl)-3(2H)-pyridazinone

¹H-NMR(400 MHz, CDCl₃); δ (ppm) 7.05-7.10(m, 2H), 7.36-7.45(m, 2H), 7.56(dd, 1H), 7.64(ddd, 1H), 7.77-7.84(m, 3H), 7.90(dd, 1H), 8.23(d, 1H), 8.78 (S, 1H), 8.88-8.92(m, 1H), 9.05(brs, 1H).

### Example 33

### 4-(2-Hydroxyphenyl)-2-phenyl-6-(2-pyridyl)-4,5-dihydro-3(2H)-pyridazinone

¹H-NMR(400 MHz, CDCl₃); δ (ppm) 3.43(dd, 1H), 4.30(dd, 1H), 6.84(td, 1H), 7.01-7.09(m, 2H), 7.32-7.37(m, 1H), 7.41-7.48(m, 3H), 7.55-7.58(m, 2H), 7.82(td, 1H), 8.20-8.22(m, 1H), 8.58(s, 1H), 8.76-8.77(m, 1H).

### Example 34

### 2-(2-Bromophenyl)-4-(2-hydroxyphenyl)-6-(3-pyridyl)-4,5-dihydro-3(2H)-pyridazinone

¹H-NMR(400 MHz, CDCl₃); δ (ppm) 3.43(dd, 1H), 3.54-3.63(m, 1H), 4.35-4.44(m, 1H), 6.82-6.88(m, 2H), 7.05(brs, 1H), 7.14(td, 1H), 7.26(td, 1H), 7.36-7.45(m, 3H), 7.66(d, 1H), 8.18(dt, 1H), 8.66(dd, 1H), 9.00(s, 1H).

The title compounds of Examples 35 to 38 were synthesized according to the method described in the above-mentioned Example 3.

### Example 35

### 2-(2-Bromophenyl)-4-(2-dimethylaminoethoxyphenyl)-6-(2-pyridyl)-3(2H)-pyridazinone

¹H-NMR(400 MHz, CDCl₃); δ (ppm) 2.25(s, 6H), 2.72(t, 2H), 4.10-4.16(m, 2H), 7.00(d, 1H), 7.03(td, 1H), 7.29-7.39(m, 3H), 7.48(td, 1H), 7.54(dd, 1H), 7.60(dd, 1H), 7.72-7.77(m, 2H), 8.13(dt, 1H), 8.62(s, 1H), 8.63-8.65(m, 1H).

### Example 36

### 2-(2-Bromophenyl)-6-(2-dimethylaminoethoxyphenyl)-4-(2-pyridyl)-3(2H)-pyridazinone

¹H-NMR(400 MHz, CDCl₃); δ (ppm) 2.24(s, 6H), 2.77-2.83(m, 2H), 4.20(t, 2H), 6.99-7.05(m, 2H), 7.30-7.41(m, 3H), 7.48(td, 1H), 7.56(dd, 1H), 7.66(dd, 1H), 8.69-8.71(m, 1H), 8.74-8.77(m, 1H), 8.95(s, 1H).

### Example 37

### 2-(2-Bromophenyl)-4-[3-(2-picolyloxyphenyl)]-6-(2-pyridyl)-3(2H)-pyridazinone

¹H-NMR(400 MHz, CDCl₃); δ (ppm) 5.2(s, 2H), 7.07(dd, 1H), 7.21-7.24(m, 1H), 7.32-7.40(m, 3H), 7.49-7.63(m, 4H), 7.70-7.79(m, 4H), 8.14(d, 1H), 8.59-8.61(m, 1H), 8.65(s, 1H), 8.67-8.69(m, 1H).

### Example 38

### 2-Phenyl-6-(2-pyridyl)-4-(2-trifluoromethylsulfonyloxyphenyl)-4,5-dihydro-3(2H)-pyrida zinone

¹H-NMR(400 MHz, CDCl₃); δ (ppm) 3.34(dd, 1H), 4.01(dd, 1H), 4.31(dd, 1H), 7.28-7.33(m, 2H), 7.36-7.48(m, 6H), 7.63(dd, 2H), 7.74(dt, 1H), 8.17(td, 1H), 8.60(ddd, 1H).

The title compounds of Examples 39 to 47 were synthesized according to the method described in the above-mentioned Example 4.

### Example 39

### 2-(2-Cyanophenyl)-4-(2-methoxyphenyl)-6-(2-pyridyl)-4,5-dihydro-3(2H)-pyridazinone

¹H-NMR(400 MHz, CDCl₃); δ (ppm) 3.50(dd, IH), 3.86(s, 3H), 3.88(dd, 1H), 4.29(dd, 1H), 6.92-6.98(m, 2H), 7.26-7.31(m, 3H), 7.43(ddd, 1H), 7.65-7.78(m, 4H), 8.17(dt, 1H), 8.57-8.59(m, 1H).

### Example 40

### 2-(2-Cyanophenyl)-4-(2-methoxyphenyl)-6-(2-pyridyl)-3(2H)-pyridazinone

¹H-NMR(400 MHz, CDCl₃); δ (ppm) 3.87(s, 1H), 7.01(d, 1H), 7.04(td, 1H), 7.33(ddd, 1H), 7.41(ddd, 1H), 7.50-7.56(m, 2H), 7.73(td, 1H), 7.76-7.81(m, 2H), 7.85(dd, 1H), 8.23(td, 1H), 8.54(s, 1H), 8.62-8.66(m, 1H).

### Example 41

### 2-(2-Cyanophenyl)-4-(2-hydroxyphenyl)-6-(2-pyridyl)-4,5-dihydro-3(2H)-pyridazinone

¹H-NMR(400 MHz, CDCl₃); δ (ppm) 3.56(dd, 1H), 4.14(dd, 1H), 4.42(dd, 1H), 6.85(td, 1H), 6.96(dd, 1H), 7.14(dd, 1H), 7.20(td, 1H), 7.38(ddd, 1H), 7.46(td, 1H), 7.58(dd, 1H), 7.70(td, 1H), 7.74-7.78(m, 2H), 8.17(dt, 1H), 8.70(dt, 1H).

### Example 42

### 2-(2-Cyanophenyl)-4-(2-hydroxyphenyl)-6-(2-pyridyl)-3(2H)-pyridazinone

¹H-NMR(400 MHz, CDCl₃); δ (ppm) 7.05-7.1 1(m, 2H), 7.38(ddd, 1H), 7.43(ddd, 1H), 7.56(dd, 1H), 7.64(ddd, 1H), 7.78-7.84(m, 3H), 7.89-7.92(m, 1H), 8.23(td, 1H), 8.24-8.71(m, 1H), 8.78(s, 1H), 9.04(s, 1H).

### Example 43

### 2-(2-Cyanophenyl)-4-phenyl-6-(2-pyridyl)-3(2H)-pyridazinone

¹H-NMR(400 MHz, CDCl₃); δ (ppm) 7.35(ddd, 1H), 7.46-7.50(m, 3H), 7.57(ddd, 1H), 7.74-7.82(m, 3H), 7.88(dd, 1H), 7.95-7.97(m, 2H), 8.23(td, 1H), 8.63(s, 1H), 8.67-8.69(m, 1H).

### Example 44

### 2-(2-Cyanophenyl)-4-(3-bromo-6-methoxyphenyl)-6-(2-pyridyl)-3 (2H)-pyridazinone

¹H-NMR(400 MHz, CDCl₃); δ (ppm) 3.85(s, 3H), 6.88(d, 1H), 7.34(ddd, 1H), 7.50(dd, 1H), 7.55(td, 1H), 7.63(d, 1H), 7.72-7.81(m, 3H), 7.86(dd, 1H), 8.22(dt, 1H), 8.53(s, 1H), 8.64-8.66(m, 1H).

### Example 45

### 2-(2-Cyanophenyl)-4-(3-pyridyl)-6-(2-pyridyl)-3(2H)-pyridazinone

¹H-NMR(400 MHz, CDCl₃); δ (ppm) 7.37(ddd, 1H), 7.42(ddd, 1H), 7.44-7.62(m, 1H), 7.78-7.83(m, 3H), 7.88(dt, 1H), 8.22(dt, 1H), 8.35(ddd, 1H), 8.67-8.71(m, 3H), 9.12(dd, 1H).

### Example 46

### 2-(2-Cyanophenyl)-4-(2-cyanophenyl)-6-(2-pyridyl)-3(2H)-pyridazinone

¹H-NMR(400 MHz, CDCl₃); δ (ppm) 7.35(ddd, 1H), 7.54-7.60(m, 2H), 7.70-7.89(m, 7H), 8.24(d, 1H), 8.65-8.67(m, 1H), 8.71(s, 1H).

### Example 47

### 4-(2-Bromophenyl)-2-(2-cyanophenyl)-6-(2-pyridyl)-3(2H)-pyridazinone

¹H-NMR(400 MHz, CDCl₃); δ (ppm) 7.32-7.39(m, 2H), 7.49-7.59(m, 3H), 7.68(td, 1H), 7.74-7.82(m, 4H), 8.14(dt, 1H), 8.65-8.67(m, 1H), 8.71(s, 1H).

The title compounds of Examples 48 to 50 were synthesized according to the method described in the above-mentioned Example 6.

### Example 48

### 2-(2-Cyanophenyl)-9-fluoro-4-phenyl-2,3,4,4a-tetrahydro-5H-(1)benzopyrano[4,3-c]-pyridazin-3-one

¹H-NMR(400 MHz, CDCl₃); δ (ppm) 3.66-3.74(m, 2H), 3.90-3.98(m, 1H), 4.03-4.09(m, 1H), 6.88(dd, 1H), 7.04(ddd, 1H), 7.28-7.33(m, 2H), 7.35-7.49(m, 4H), 7.64-7.77(m, 4H).

### Example 49

### 2-(2-Cyanophenyl)-4-(3 -pyridyl)-2,3,4,4a-tetrahydro-5H-(1) benzopyrano[4,3-c]pyridazin-3-one

¹H-NMR(400 MHz, CDCl₃); δ (ppm) 3.66-3.80(m, 2H), 3.97-4.10(m, 2H), 6.91-6.95(m, 1H), 7.00-7.06(m, 1H), 7.32-7.37(m, 1H), 7.41-7.51(m, 2H), 7.66-7.79(m, 4H), 8.01-8.06(m, 1H), 8.50-8.53(m, 1H), 8.64-8.68(m, 1H).

### Example 50

### 2-(2-Bromophenyl)-4-(3-pyridyl)-2,3,4,4a-tetrahydro-5H-(1)benzopyrano[4,3-c]pyridazin-3-one

¹H-NMR(400 MHz, CDCl₃); δ (ppm) 3.64-3.77(m, 1H), 3.93(d, 1H), 4.01-4.12(m, 2H), 6.94(d, 1H), 6.98-7.04(m, 1H), 7.28-7.38(m, 2H), 7.42-7.58(m, 2H), 7.66-7.74(m, 2H), 7.98(dd, 1H), 8.01-8.07(m, 1H), 8.67-8.74(m, 2H).

The title compounds of Examples 51 to 61 were synthesized according to the method described in the above-mentioned Example 7.

### Example 51

### 2-(2-Bromophenyl)-4-(2-hydroxyphenyl)-6-(2-pyridyl)-3(2H)-pyridazinone

¹H-NMR(400 MHz, CDCl₃); δ (ppm) 7.04-7.09(m, 2H), 7.35-7.45(m, 3H), 7.54-7.59(m, 3H), 7.76-7.81(m, 2H), 8.17(dt, IH), 8.67-8.71(m, 1H), 8.79(s, 1H), 9.36(s, 1H).

### Example 52

### 2-(2-Bromophenyl)-4-(3-methoxyphenyl)-6-(2-pyridyl)-3(2H)-pyridazinone

¹H-NMR(400 MHz, CDCl₃); δ (ppm) 3.86(s, 3H), 6.98(d, 2H), 7.31-7.38(m, 2H), 7.48-7.56(m, 2H), 7.73-7.78(m, 2H), 8.04(d, 2H), 8.14(dt, 1H), 8.60(s, 1H), 8.67-8.68(m, 1H).

### Example 53

### 2-(2-Cyanophenyl)-9-fluoro-5-hydroxy-4-phenyl-2,3 -dihydro-5H-(1)benzopyrano[4,3-c]pyridazin-3-one

¹H-NMR(400 MHz, CDCl₃); δ (ppm) 3.61(d, 1H), 6.10(d, 1H), 7.01(dd, 1H), 7.07-7.14(m, 1H), 7.47-7.54(m, 3H), 7.54-7.66(m,3H), 7.72-7.80(m, 3H), 7.83-7.88(m, 1H).

### Example 54

### 2-(2-Cyanophenyl)-9-fluoro-4-phenyl-2,3-dihydro-5H-( I )benzopyrano[4,3-c]pyridazin-3-one

¹H-NMR(400 MHz, CDCl₃); δ (ppm) 5.09(s, 2H), 6.96(dd, 1H), 7.02-7.09(m, 1H), 7.37-7.43(m, 2H), 7.45-7.60(m, 4H), 7.72(dd, IH), 7.74-7.79(m, 2H), 7.86(d, 1H).

### Example 55

### 2-Phenyl-6-(2-pyridyl)-4-(2-trifluoromethylsulfonyloxyphenyl)-3(2H)-pyridazinone

¹H-NMR(400 MHz, CDCl₃); δ (ppm) 7.33(ddd, 1H), 7.41-7.45(m, 2H), 7.49-7.55(m, 4H), 7.65(dd, 1H), 7.73-7.55(m, 1H), 7.79(dt, 1H), 8.21(td, 1H), 8.57(s, 1H), 8.65(ddd, 1H).

### Example 56

### 2-(2-Bromophenyl)-4-phenyl-6-(2-pyridyl)-3(2H)-pyridazinone

¹H-NMR(400 MHz, CDCl₃); δ (ppm) 7.32(ddd, 1H), 7.36(ddd, 1H), 7.44-7.50(m, 4H), 7.54(td, 1H), 7.73-7.78(m, 2H), 7.99-8.01(m, 2H), 8.14(dt, 1H), 8.65(s, 1H), 8.66-8.68(m, 1H).

### Example 57

### 2-(2-Bromophenyl)-4-(3-pyridyl)-2,3-dihydro-5H-(1)benzopyrano[4,3-c]pyridazine-3-one

¹H-NMR(400 MHz, DMSO-d₆); δ (ppm) 5.12-5.28(m, 2H), 7.06-7.15(m,2H), 7.43(ddd, 1H), 7.49(ddd, 1H), 7.55-7.64(m, 2H), 7.71(dd, 1H), 7.84-7.93(m, 3H), 8.64(d, 1H), 8.68(dd, 1H).

### Example 58

### 2-(2-Iodophenyl)-4-(3 -pyridyl)-2,3-dihydro-5H-(1)benzopyrano[4,3-c]pyridazin-3-one

¹H-NMR(400 MHz, DMSO-d₆); δ (ppm) 5.15-5.28(m, 2H), 7.06-7.16(m, 2H), 7.27-7.33(m, 1H), 7.40-7.46(m, 1H), 7.55-7.67(m, 3H), 7.86(dd, 1H), 7.90(ddd, 1H), 8.06(dd, 1H), 8.64(d, 1H), 8.69(dd, 1H).

### Example 59

### 2-Phenyl-4-(3-pyridyl)-6-(2-pyridyl)-3(2H)-pyridazinone

¹H-NMR(400 MHz, CDCl₃); δ (ppm) 7.35(ddd, 1H), 7.40-7.48(m, 1H), 7.52-7.56(m, 2H), 7.71-7.75(m, 2H), 7.80(td, 1H), 8.20(dt, 1H), 8.37(dt, 1H), 8.67-8.69(m, 3H), 9.12(d, 1H).

### Example 60

### 4-(2-Bromophenyl)-2-phenyl-6-(2-pyridyl)-3(2H)-pyridazinone

¹H-NMR(400 MHz, CDCl₃); δ (ppm) 7.27-7.53(m, 7H), 7.69(dd, 1H), 7.77-7.81(m, 3H), 8.22(dt, 1H), 8.47(s, 1H), 8.63-8.65(m, 1H).

### Example 61

### 2-(2-Bromophenyl)-4-(3-pyridyl)-6-(2-pyridyl)-3(2H)-pyridazinone

¹H-NMR(400 MHz, CDCl₃); δ (ppm) 7.33-7.42(m, 3H), 7.52(td, 1H), 7.55(dd, 1H), 7.77(td, 2H), 8.15(dt, 1H), 8.18(dt, 1H), 8.67-8.69(m, 2H), 8.72(s, 1H), 9.15(dd, 1H).

### Example 62

### 2-(2-Chlorophenyl)-4-(4-morpholinoethylaminocarbonyl)-6-phenyl-3(2H)-pyridazinone

The title compound was synthesized according to the method described in the above-mentioned Example 12.

¹H-NMR(400 MHz, CDCl₃); δ (ppm) 2.45-2.64(m, 6H), 3.58-3.76(m, 6H), 7.46-7.53(m, 6H), 7.59-7.63(m, 1H), 7.86-7.91(m, 2H), 8.84(s, 1H), 9.64(brs, 1H).

### Example 63

### 2-(2-Nitrophenyl)-4-(3-pyridyl)-6-(2-pyridyl)-3(2H)- pyridazinone

2-(3-Pyridyl)-4-(2-pyridyl)-4-oxobutyric acid (100 mg) was dissolved in 1-butanol (5 ml), 2-nitrophenylhydrazine (60 mg) was added thereto, and the mixture was heated under reflux for 3 hours. After cooling as it was to room temperature, it was evaporated. The residues was dissolved in acetic acid (5 ml) and heated under reflux overnight. After cooling as it was to room temperature, it was evaporated. The residue was diluted with ethyl acetate and washed with an aqueous saturated sodium bicarbonate solution and brine. The organic layer was dried over magnesium sulfate, evaporated and purified by silica gel column chromatography (ethyl acetate), to give the title compound (20 mg).

¹H-NMR (400 MHz, CDCl₃); δ (ppm) 7.34-7.42 (m, 2H), 7.66 (ddd, 1H), 7.74-7.85 (m, 3H), 8.10 (ddd, 1H), 8.17 (dd, 1H), 8.32 (ddd, 1H), 8.67-8.71 (m, 2H), 8.72 (s, 1H), 9.10 (dd, 1H).

### Example 64

### 2-(3 -Tolyl)-4-(3-pyridyl)-6-(2-pyridyl)-3(2H)-pyridazinone

### 64-1) 4-(3-Pyridyl)-6-(2-pyridyl)-4,5-dihydro-3(2H)-pyridazinone

2-(3-Pyridyl)-4-(2-pyridyl)-4-oxobutyric acid (1.88 g) was dissolved in ethanol (40 ml) and hydrazine monohydrate (0.37 g) was added thereto, followed by heating under reflux overnight. After cooling as it was to room temperature, the mixture was evaporated. The residue was diluted with methylene chloride, and washed with water and brine. The organic layer was dried over magnesium sulfate, and then evaporated and purified by silica gel column chromatography (methanol-chloroform), to give the title compound (1.77 g).

¹H-NMR (400 MHz, CDCl₃); δ (ppm) 3.45(dd, 1H), 3.71(dd, 1H), 3.88(dd, 1H), 7.27-7.34(m, 2H), 7.66(ddd, 1H), 7.76(ddd, IH), 8.05(ddd, 1H), 8.55(dd, 1H), 8.57-8.62(m, 2H), 8.78(br s, 1H).

### 64-2) 2-(3-Tolyl)-4-(3-pyridyl)-6-(2-pyridyl)-3(2H)-pyridazinone

4-(3-Pyridyl)-4-(2-pyridyl)-4,5-dihydro-3(2H)-pyridazinone (50 mg) was dissolved in N,N-dimethylformamide (2 ml), and m-tolylboronic acid (54 mg), triethylamine (0.06 ml) and copper acetate (7 mg) were added thereto, followed by stirring at room temperature for 1 day. The reaction solution was diluted with ethyl acetate and washed with aqueous ammonia and brine. The organic layer was dried over magnesium sulfate, and then evaporated and purified by NH silica gel column chromatography (ethyl acetate-hexane), to give the title compound (10 mg).

¹H-NMR (400 MHz, CDCl₃); δ (ppm) 2.46 (s, 3H), 7.26-7.30 (m, 1H), 7.35 (ddd, 1H), 7.38-7.45 (m, 2H), 7.47-7.54 (m, 2H), 7.80 (ddd, 1H), 8.20 (ddd, 1H), 8.36 (ddd, 1H), 8.66 (s, 1H), 8.67-8.70 (m, 2H), 9.10-9.12 (m, 1H).

The title compounds of Examples 65 to 69 were synthesized according to the method described in the above-mentioned Example 64.

### Example 65

### 2-(4-Methanesulfonylphenyl)-4-(3-pyridyl)-6-(2-pyridyl)-3(2H)-pyridazinone

¹H-NMR (400 MHz, CDCl₃); δ (ppm) 3.12 (s, 3H), 7.40 (ddd, 1H), 7.43 (dd, 1H), 7.84 (ddd, 1H), 8.02-8.07 (m, 2H), 8.10-8.15 (m,2H), 8.20 (d, 1H), 8.32 (ddd, 1H), 8.69-8.73 (m, 3H), 9.11 (d, 1H).

### Example 66

### 2-(4-Biphenyl)-4-(3-pyridyl)-6-(2-pyridyl)-3(2H)-pyridazinone

¹H-NMR (400 MHz, CDCl₃); δ (ppm) 7.34-7.44 (m, 3H), 7.46-7.51 (m, 2H), 7.63-7.668 (m, 2H), 7.73-7.77 (m, 2H), 7.79-7.84 (m,3H), 8.24 (dd, 1H), 8.37 (ddd, 1H), 8.68-8.71 (m, 3H), 9.13 (d, 1H).

### Example 67

### 2-(2-Naphthyl)-4-(3-pyridyl)-6-(2-pyridyl)-3(2H)-pyridazinone

¹H-NMR (400 MHz, CDCl₃); δ (ppm) 7.36 (ddd, 1H), 7.42 (dd, 1H), 7.53-7.60 (m, 2H), 7.78-7.86 (m, 2H), 7.90-7.96 (m, 2H), 8.00 (d, 1H), 8.22-8.27 (m, 2H), 8.38 (ddd, 1H), 8.68-8.73 (m, 3H), 9.14 (d, 1H).

### Example 68

### 2-(3,4-Methylenedioxyphenyl)-4-(3-pyridyl)-6-(2-pyridyl)- 3(2H)-pyridazinone

¹H-NMR (400 MHz, CDCl₃); δ (ppm) 6.07 (s, 2H), 6.93-6.96 (m, 1H), 7.17-7.21 (m, 2H), 7.36 (dd, 1H), 7.41 (dd, 1H), 7.80 (ddd, 1H), 8.18 (d, 1H), 8.34 (ddd, 1H), 8.65 (s, 1H), 8.67-8.71 (m, 2H), 9.11 (d, 1H).

### Example 69

### 2-(3,4-Dichlorophenyl)-4-(3-pyridyl)-6-(2-pyridyl)-3(2H)-pyridazinone

¹H-NMR (400 MHz, CDCl₃); δ (ppm) 7.38 (ddd, 1H), 7.42 (ddd, 1H), 7.60 (d, 1H), 7.68 (dd, 1H), 7.83 (ddd, 1H), 7.93 (d, 1H), 8.19 (ddd, 1H), 8.32 (ddd, 1H), 8.67 (s, 1H), 8.68-8.72 (m, 2H), 9.08 (d, 1H).

### Example 70

### 2-(2-Cyanophenyl)-4-phenyl-6-(2-pyrimidinyl)-3(2H)-pyridazinone

4-Phenyl-6-(2-pyrimidinyl)-3(2H)-pyridazinone (100 mg) was dissolved in methylene chloride (5 ml), and 2-(2-cyanophenyl)-1,3,2-dioxaborinate (0.22 g), pyridine (0.10 g) and copper acetate (0.15 g) were added thereto, followed by stirring at room temperature for 1 day. The reaction solution was diluted with methylene chloride, and washed with aqueous ammonia, water and brine. The organic layer was dried over magnesium sulfate and then evaporated. The residue was purified by NH silica gel column chromatography (ethyl acetate-hexane), to give the title compound (29 mg).

¹H-NMR (400 MHz, CDCl₃); δ (ppm) 7.37(dd, 1H), 7.46-7.53(m, 3H), 7.57(ddd, 1H), 7.72-7.80(m, 2H), 7.84(dd, 1H), 7.95-8.00(m, 2H), 8.70(s, 1H), 8.92(d, 2H).

### Example 71

### 2-(2-Pyridyl)-4-(2-pyridyl)-6-(2-methoxyphenyl)-3(2H)-pyridazinone

The title compound was synthesized according to the method described in the above-mentioned Example 5.

¹H-NMR (400 MHz, CDCl₃); δ (ppm) 3.89(s, 3H), 6.98-7.04(m, 2H), 7.31-7.42(m, 4H), 7.62(dd, 1H), 7.68(dt, 1H), 7.78(ddd, 1H), 7.91(ddd, 1H), 8.70-8.73(m, 3H).

The title compounds of Examples 72 to 75 were synthesized according to the method described in the above-mentioned Example 6.

### Example 72

### 2-(3-Formylphenyl)-4-(3-pyridyl)-6-(2-pyridyl)-3(2H)-pyridazinone

¹H-NMR (400 MHz, CDCl₃); δ (ppm) 7.37-7.44(m, 2H), 7.72(t, 1H), 7.83(dt, 1H), 7.98(td, 1H), 8.08(ddd, 1H), 8.21(td, 1H), 8.30-8.36(m, 2H), 8.69-8.72(m, 3H), 9.10-9.12(m, 1H), 10.11 (s, 1H).

### Example 73

### 2-(Thiophen-3-yl)-4-(3-pyridyl)-6-(2-pyridyl)-3(2H)-pyridazinone

¹H-NMR (400 MHz, CDCl₃); δ (ppm) 7.36-7.44(m, 3H), 7.77(dd, 1H), 7.82(ddd, 1H), 8.20(dd, 1H), 8.26(td, 1H), 8.32(ddd, 1H), 8.61 (s, 1H), 8.68-8.71 (m, 2H), 9.08(dd, 1H).

### Example 74

### 2-(3-Pyridyl)-4-phenyl-6-(2-pyridyl)-3(2H)-pyridazinone

¹H-NMR (400 MHz, CDCl₃); δ (ppm) 7.36(ddd, 1H), 7.44-7.52(m, 4H), 7.82(dt, 1H), 7.92-7.95(m, 2H), 8.18-8.22(m, 2H), 8.63(s, 1H), 8.66(dd, 1H), 8.69(ddd, 1H), 9.06(d, 1H).

### Example 75

### 2-(3-Pyridinyl)-4-phenyl-6-(2-pyrimidinyl)-3(2H)-pyridazinone

¹H-NMR (400 MHz, CDCl₃); δ (ppm) 7.38(t, 1H), 7.44-7.52(m, 4H), 7.94-7.96(m, 2H), 8.11(ddd, 1H), 8.64(s, 1H), 8.67(dd, 1H), 8.93(d, 2H), 9.02(dd, 1H).

The title compounds of Examples 76 to 78 were synthesized according to the method described in the above-mentioned Example 1.

### Example 76

### 2-(2-Methoxyphenyl)-4-(3-pyridyl)-6-(2-pyridyl)-4,5-dihydro-3(2H)-pyridazinone

¹H-NMR (400 MHz, CDCl₃); δ (ppm) 3.56-3.88(m, 5H), 4.10(t, 1H), 7.00-7.07(m, 2H), 7.25-7.29(m, 2H), 7.34-7.40(m, 2H), 7.67(dt, 1H), 7.75-7.80(m, 1H), 8.05(td, 1H), 8.52(dd, 1H), 8.60(ddd, 1H), 8.65-8.69(m, 1H).

### Example 77

### 4-Methyl-2,4,6-triphenyl-4,5-dihydro-3(2H)-pyridazinone

¹H-NMR (400 MHz, CDCl₃); δ (ppm) 1.70(s, 3H), 3.11 (d, 1H), 3.72(d, 1H), 7.22-7.30(m, 4H), 7.33-7.36(m, 2H), 7.38-7.42(m, 5H), 7.53-7.57(m, 2H), 7.75-7.78(m, 2H).

### Example 78

### 2-(2-Bromophenyl)-4-methyl-4,6-diphenyl-4,5-dihydro-3(2H)-pyridazinone

¹H-NMR (400 MHz, CDCl₃); δ (ppm) 1.73(s, 3H), 3.17(d, 1H), 3.75(d, 1H), 7.22-7.25(m, 2H), 7.28-7.32(m, 2H), 7.36-7.42(m, 7H), 7.64-7.68(m, 2H), 7.72-7.78(m, 2H).

### Example 79

### 2-(3-Pyridin-1-oxide)-4-phenyl-6-(2-pyridyl)-3(2H)-pyridazinone

70% m-chloroperbenzoic acid (1.27 g) was added little by little to a solution of 2-(3-pyridyl)-4-phenyl-6-(2-pyridyl)-3(2H)-pyridazinone (224 mg) in dichloromethane (5 ml) under ice-cooling, followed by stirring for 1 hour. The reaction mixture was partitioned between 2 N aqueous sodium hydroxide and ethyl acetate, and the organic layer was washed with water, dried and concentrated. The residue was purified by NH-silica gel column chromatography (ethyl acetate), to give the title compound (117 mg) as a pale yellow solid.

¹H-NMR (400 MHz, CDCl₃); δ (ppm) 7.38-7.42(m, 2H), 7.48-7.52(m, 3H), 7.84(dt, 1H), 7.89-7.91(m, 2H), 7.99(ddd, 1H), 8.21(td, 1H), 8.25(ddd, 1H), 8.62(s, 1H), 8.69(ddd, 1H), 8.84(t, 1H).

### Example 80

### 80A) 2-(2-Cyanopyridin-5-yl)-4-phenyl-6-(2-pyridyl)-3(2H)-pyridazinone

### 80B) 2-(2-Cyanopyridin-3-yl)-4-phenyl-6-(2-pyridyl)-3(2H)-pyridazinone

Cyanotrimethylsilane (0.12 ml) and triethylamine (84 μl) were added to a solution of 2-(3-pyridine-1-oxide)-4-phenyl-6-(2-pyridyl)-3(2H)-pyridazinone (52 mg) in a mixed solvent of acetonitrile (2 ml) and chloroform (2 ml), followed by heating under reflux overnight. After concentrating the reaction mixture, the residue was purified by NH-silica gel column chromatography (chloroform/hexane system), to give 2-(2-cyanopyridin-5-yl)-4-phenyl-6-(2-pyridyl)-3(2H)-pyridazinone (24 mg) as a colorless solid and 2-(2-cyanopyridin-3-yl)-4-phenyl-6-(2-pyridyl)-3(2H)-pyridazinone (12 mg) as a yellow solid.

80A;

¹H-NMR (400 MHz, CDCl₃); δ (ppm) 7.40(ddd, 1H), 7.49-7.52(m,3H), 7.83-7.92(m, 4H), 8.19(td, 1H), 8.64(s, 1H), 8.70(ddd, 1H), 9.27(dd, 1H).

80B;

¹H-NMR (400 MHz, CDCl₃); δ (ppm) 7.38(ddd, 1H), 7.48-7.52(m, 3H), 7.70(dd, 1H), 7.82(dt, 1H), 7.93-7.95(m, 2H), 8.22(dd, 1H), 8.26(td, 1H), 8.66(s, 1H), 8.69(ddd, 1H), 8.79(dd, 1H).

### Example 81

### 81A) 2-(2-Cyanopyridin-5-yl)-4-phenyl-6-(2-pyrimidyl)-3(2H)-pyridazinone

### 81B) 2-(2-Cyanopyridin-3-yl)-4-phenyl-6-(2-pyrimidyl)-3(2H)-pyridazinone

2-(2-Cyanopyridin-5-yl)-4-phenyl-6-(2-pyrimidyl)-3(2H)-pyridazinone and 2-(2-cyanopyridin-3-yl)-4-phenyl-6-(2-pyrimidyl)-3(2H)-pyridazinone were obtained in the same manner as in the above-mentioned Example 80.

81A;

¹H-NMR (400 MHz, CDCl₃); δ (ppm) 7.42(t, 1H), 7.50-7.54(m, 3H), 7.85(dd, 1H), 7.91-7.93(m, 2H), 8.37(dd, 1H), 8.63(s, 1H), 8.94(d, 2H), 9.23(dd, 1H).

81B;

¹H-NMR (400 MHz, CDCl₃); δ (ppm) 7.40(t, 1H), 7.49-7.52(m, 3H), 7.71(dd, 1H), 7.95-7.97(m, 2H), 8.12(dd, 1H), 8.71(s, 1H), 8.79(dd, 1H), 8.93(d, 2H).

### Example 82

### 2-(2-Cyanophenyl)-4-phenyl-6-(2-pyrazyl)-3(2H)-pyridazinone

6-Chloro-2-(2-cyanophenyl)-4-phenyl-3 (2H)-pyridazinone (16 mg), 2-tributylstannyl pyrazine (25 mg) and tetrakis(triphenylphosphine) palladium (3 mg) were added to xylene (1 ml), followed by stirring at 120° C. for 2 hours in a nitrogen atmosphere. The reaction mixture was purified by NH-silica gel column chromatography (ethyl acetate/hexane system), to give the title compound (14 mg) as a pale yellow solid.

¹H-NMR (400 MHz, CDCl₃); δ (ppm) 7.45-7.62(m, 4H), 7.78-7.80(m, 2H), 7.88-7.96(m, 3H), 8.54(s, 1H), 8.62-8.64(m, 2H), 9.45(d,2H).

### Example 83

### 2-(2-Cyanophenyl)-4-phenyl-6-(thiazol-2-yl)-3(2H)-pyridazinone

The title compound was synthesized according to the above-mentioned Example 82.

¹H-NMR (400 MHz, CDCl₃); δ (ppm) 7.47-7.50(m, 4H), 7.59(ddd,1H), 7.75-7.81(m,2H), 7.87(ddd,1H), 7.92-7.95(m, 3H), 8.40(s, 1H).

### Example 84

### 2-(2-Cyanophenyl)-4-methyl-4,6-diphenyl-4,5-dihydro-3(2H)-pyridazinone

The title compound was synthesized according to the above-mentioned Example 4.

¹H-NMR (400 MHz, CDCl₃); δ (ppm) 1.75(s, 3H), 3.21(d, 1H), 3.74(d, 1H), 7.25-7.27(m, 1H), 7.29-7.34(m, 2H), 7.37-7.45(m,6H), 7.57(ddd, 1H), 7.66(ddd, 1H), 7.73(ddd, 1H), 7.75-7.78(m, 2H).

### Example 85

### 2-(2-Bromophenyl)-4-(2-methoxyphenyl)-6-(2-pyridyl)-4,5-dihydro-1,2,4-triazin-3(2H)-o ne

2-(2-Bromophenyl)-6-(2-pyridyl)-4,5-dihydro-1,2,4-triazin-3(2H)-one (70 mg) was dissolved in tetrahydrofuran anhydride (10 ml), and copper acetate (77 mg), sodium hydride (25 mg) and 2-methoxyphenylboronic acid (77 mg) were added thereto, followed by stirring at room temperature for 1 hour. Sodium hydride (25 mg) and 2-methoxyphenylboronic acid (50 mg) were further added thereto and stirred at room temperature for 6 hours. Then, the organic layer was partitioned by adding ethyl acetate and aqueous ammonia thereto. The organic layer was washed with water and dried over anhydrous sodium sulfate. After the drying agent was filtered off, the filtrate was evaporated. The residue was purified by silica gel column chromatography (hexane-ethyl acetate system), to give the title compound (55 mg, 60%) as a colorless powder.

¹H-NMR (400 MHz, CDCl₃); δ (ppm) 3.85 (s, 3H), 5.02 (s, 2H), 6.94-6.99 (m, 2H), 7.20-7.31 (m, 3H), 7.35-7.42 (m, 2H), 7.62 (dd, J=7.6 Hz, 1.6 Hz, 1H), 7.66-7.73 (m, 2H), 8.09-8.11 (m, 1H), 8.54 (ddd. J=5.0 Hz, 1.8 Hz, 1.6 Hz, 1H).

### Example 86

### 2-(2-Bromophenyl)-4-(2-hydroxyphenyl)-6-(2-pyridyl)-4,5-dihydro- 1,2,4-triazin-3(2H)-one

1 M boron tribromide in methylene chloride (0.3 ml) was added to a solution of 2-(2-bromophenyl)-4-(2-methoxy phenyl)-6-(2-pyridyl)-4,5-dihydro-1,2,4-triazin-3(2H)-one (48 mg) in dichloromethane (10 ml) under ice-cooling and stirred for 5 hours. The organic layer was partitioned by adding an aqueous saturated sodium bicarbonate solution and dichloromethane to the reaction solution, washed with water and dried over anhydrous sodium sulfate. After the drying agent filtered off, the filtrate was evaporated. The residue was purified by silica gel column chromatography (hexane-ethyl acetate system), to give the title compound (36 mg, 78%) as a colorless amorphous.

¹H-NMR (400 MHz, CDCl₃); δ (ppm) 5.23 (brs, 2H), 7.02-7.10 (m, 2H), 7.20-7.36 (m, 4H), 7.46 (td, J=8.0 Hz, 1.2 Hz, 1H), 7.62 (dd, J=8.0 Hz, 1.8 Hz, 1H), 7.71-7.76 (m, 2H), 8.09 (d, J=8.0 Hz, 1H), 8.59 (d, J=5.2 Hz, 1H).

ESI-Mass; 423 [M⁺+H]

### Example 87

### 2-(2-Cyanophenyl)-4-(2-methoxyphenyl)-6-(2-pyridyl)-4,5-dihydro-1,2,4-triazin-3(2H)-on e

2-(2-Bromophenyl)-4-(2-methoxyphenyl)-6-(2-pyridyl)-4,5-dihydro-1,2,4-triazin-3(2H)-one (99 mg) was dissolved in dimethylformamide (20 ml), and copper cyanide (51 mg) was added thereto, followed by stirring at 150° C. for 3 hours. After cooling the reaction solution to room temperature, the organic layer was partitioned by adding aqueous ammonia (20 ml) and ethyl acetate thereto. The organic layer was washed with water and dried over anhydrous sodium sulfate. After the drying agent was filtered off, the residue was purified by silica gel column chromatography (hexane-ethyl acetate system), to give the title compound (72 mg, 83%) as a colorless amorphous.

¹H-NMR (400 MHz, CDCl₃); δ (ppm) 3.87 (s, 3H), 5.04 (s, 2H), 6.97-7.01 (m, 2H), 7.25-7.39 (m, 4H), 7.61-7.66 (m, 1H), 7.72-7.78 (m, 3H), 8.22 (d, J=8.0 Hz, 1H), 8.54 (ddd, J=4.8 Hz, 1.6 Hz, 1.2 Hz, 1H).

ESI-Mass; 384 [M⁺+H]

### Example 88

### 2-(2-Bromophenyl)-4-phenyl-6-(2-pyridyl)-4,5-dihydro-1,2,4-triazin-3(2H)-one

2-(2-Bromophenyl)-6-(2-pyridyl)-4,5-dihydro-1,2,4-triazin-3(2H)-one (12 mg) was dissolved in anhydrous dichloromethane (30 ml), and triethylamine (0.1 ml), copper acetate (13.2 mg) and phenylboronic acid (13.3 mg) were added thereto, followed by stirring at room temperature for 48 hours. Sodium hydride (3 mg) and phenylboronic acid (10 mg) were further added thereto and stirred at room temperature for 5 hours. Then, the organic layer was partitioned by adding aqueous ammonia and ethyl acetate thereto. The organic layer was washed with water and dried over anhydrous sodium sulfate. After the drying agent was filtered off, the residue was purified by NH silica gel column chromatography (hexane-ethyl acetate system), to give the title compound (11 mg, 75%) as a colorless powder.

¹H-NMR (400 MHz, CDCl₃); δ (ppm) 5.15 (brs, 2H), 7.21-7.31 (m, 3H), 7.36-7.46 (m 3H), 7.47-7.52 (m, 2H), 7.62 (dd, J=8.0 Hz, 1.8 Hz, 1H), 7.68-7.74 (m, 2H), 8.10 (dt, J=8.0 Hz, 1.4 Hz, 1H), 8.57 (ddd, J=5.0 Hz, 1.8 Hz, 0.8 Hz, 1H).

ESI-Mass; 407[M⁺+H]

### Example 89

### 2-(2-Bromophenyl)-6-(2-methoxyphenyl)-4-phenyl-4,5-dihydro-1,2,4-triazin-3(2H)-one

According to the method of Horst Gnichtel, Widah I. Salem und Lothar Waretschek; Liebigs Ann. Chem. (1978) 2033-2043, the synthesis was carried out as follows. 2-Bromo-2′-methoxyacetophenone (1.33 g) and aniline (1.06 g) were dissolved in ethanol (20 ml) and stirred at room temperature for 72 hours. After the insoluble matters were filtered off, the filtrate was evaporated. The residue was purified by silica gel column chromatography (hexane-ethyl acetate system). The resulting colorless oily acetophenone derivative (1.12 g) was dissolved in ethanol (20 ml), and 2-bromophenyl hydrazine (930 mg) was added thereto, followed by stirring at room temperature overnight. The reaction solution was evaporated, and the residue (1.84 g) was dissolved in tetrahydrofuran (20 ml). Triphosgene (459 mg) and triethylamine (1.4 ml) were added thereto under ice-cooling, and the mixture was stirred for 3 hours while the temperature of the mixture was gradually raised to room temperature. The reaction solution was evaporated, and the residue was purified by silica gel column chromatography (hexane-ethyl acetate), to give the title compound (438 mg, 17%) as a colorless amorphous.

¹H-NMR (400 MHz, CDCl₃); δ (ppm) 3.87 (s, 3H), 4.85 (s, 2H), 6.92 (d, J=8.4 Hz, 1H), 7.01 (t, J=7.4 Hz, 1H), 7.18-7.25 (m, 2H), 7.27-7.46 (m, 6H), 7.64-7.68 (m, 2H), 7.71 (dt, J=7.6 Hz, 1.6 Hz, 1H).

ESI-Mass; 436 [M⁺+H]

### Example 90

### 2-(2-Bromophenyl)-6-(2-hydroxyphenyl)-4-phenyl-4,5 -dihydro- 1,2,4-triazin-3 (2H)-one

According to the method described in Example 86, the title compound (135 mg, 95%) was obtained form 2-(2-bromophenyl)-6-(2-methoxyphenyl)-4-phenyl-4,5-dihydro-1,2,4-triazin-3(2H)-one (147 mg).

¹H-NMR (400 MHz, CDCl₃); δ (ppm) 4.98 (s, 2H), 6.91 (t, J=8.0 Hz, 1H), 7.00 (dd, J=8.0 Hz, 0.8 Hz, 1H), 7.25-7.35 (m, 4H), 7.40-7.48 (m, 5H), 7.58 (dd, J=8.0 Hz, 1.6 Hz, 1H), 7.69 (dd, J=7.8 Hz, 1.4 Hz, 1H), 10.96 (s, 1H).

ESI-Mass; 424 [M⁺+H]

Example 91

### 2-(2-Bromophenyl)-6-(2-dimethylaminoethoxyphenyl)-4-phenyl-4,5-dihydro-1,2,4-triazin-3(2H)-one

2-(2-Bromophenyl)-6-(2-hydroxyphenyl)-4-phenyl-4,5-dihydro-1,2,4-triazin-3(2H)-one (100 mg) was dissolved in dimethylformamide (20 ml), and potassium carbonate (66 mg) was added thereto. An excess amount of dimethylaminoethyl chloride was added dropwise thereto and stirred at 120° C overnight. After cooling the reaction solution to room temperature, the organic layer was partitioned by adding water and ethyl acetate thereto. The organic layer was washed with water and dried over anhydrous sodium sulfate. After the drying agent was filtered off, the filtrate was evpaorated. The residue was purified by silica gel column chromatography (hexane-ethyl acetate system), to give the title compound (65 mg, 55%) as colorless crystals.

¹H-NMR (400 MHz, CDCl₃); δ (ppm) 2.26 (s, 6H), 2.65 (t, J=5.8 Hz, 2H), 4.11 (t, J=5.8 Hz, 2H), 4.93 (s, 2H), 6.91 (d, J=8.0 Hz, 1H), 6.98 (td, J=7.6 Hz, 0.8 Hz, 1H), 7.16-7.25 (m, 2H), 7.33-7.39 (m, 3H), 7.40-7.46 (m, 3H), 7.66 (td, J=8.0 Hz, 1.6 Hz, 2H), 7.72 (dd, J=7.8 Hz, 1.8 Hz, 1H).

ESI-Mass; 495 [M⁺+H]

### Example 92

### 2-(2-Bromophenyl)-6-(2-methoxyphenyl)-4-(2-pyridyl)-4,5-dihydro-1,2,4-triazin-3(2H)-o ne

2-(2-Bromophenyl)-6-(2-methoxyphenyl)-4,5-dihydro-1,2,4-triazin-3(2H)-one (193 mg) synthesized according to the method described in Examples 85 to 87 was dissolved in dimethylformamide (20 ml), and 2-bromopyridine (300 mg), potassium carbonate (185 mg) and copper iodide (20.4 mg) were added thereto, followed by heating at 130° C. for 5 hours. After cooling to room temperature, the organic layer was partitioned by adding aqueous ammonia (20 ml) and ethyl acetate thereto. The organic layer was washed with water and dried over anhydrous sodium sulfate. After the drying agent was filtered off, the filtrate was evaporated. The residue was purified by NH silica gel column chromatography (hexane-ethyl acetate system), to give the title compound (126 mg, 54%) as a colorless powder.

¹H-NMR (400 MHz, CDCl₃); δ (ppm) 3.84 (s, 3H), 5.10 (brs, 2H), 6.86-6.97 (m, 3H), 7.14-7.18 (m, 1H), 7.29-7.38 (m, 2H), 7.54-7.63 (m, 4H), 7.92-7.95 (m, 1H), 8.29-8.31 (m, 1H).

ESI-Mass; 437 [M⁺+H]

The title compounds of Examples 93 to 96 were synthesized according to the method described in the above-mentioned Example 86.

### Example 93

### 2-(2-Cyanophenyl)-6-(2-hydroxyphenyl)-4-phenyl-4,5-dihydro-1,2,4-triazin-3(2H)-one

¹H-NMR (400 MHz, CDCl₃); δ (ppm) 5.00 (s, 2H), 6.91-6.96 (m, 1H), 7.02 (dd, J=8.4 Hz, 0.8 Hz, 1H), 7.27-7.49 (m, 8H), 7.70-7.73 (m, 2H), 7.73-7.77 (m, 1H), 10.83 (s, 1H).

ESI-Mass; 369 [M⁺+H]

### Example 94

### 2-(2-Bromophenyl)-4-(2,5-dihydroxyphenyl)-6-(2-hydroxyphenyl)-4,5-dihydro-1,2,4-triaz in-3(2H)-one

¹H-NMR (400 MHz, DMSO-d₆); δ (ppm) 4.79 (s, 2H), 6.59 (dd, J=8.8 Hz, 2.8 Hz, 1H), 6.70-6.74 (m, 2H), 6.89 (dd, J=13.2 Hz, 0.8 Hz, 2H), 7.28-7.37 (m, 2H), 7.49-7.57 (m, 2H), 7.66 (dd, J=7.8 Hz, 1.0 Hz, 1H), 7.76 (d, J=8.2 Hz, 1H), 8.92 (s, 1H), 9.09 (brs, 1H), 10.52 (brs, 1H).

ESI-Mass; 454 [M⁺+H]

### Example 95

### 4-(2,5-Dihydroxyphenyl)-6-(2-hydroxyphenyl)-2-phenyl-4,5-dihydro-1,2,4-triazin-3(2H)-one

¹H-NMR (400 MHz, DMSO-d₆); δ (ppm) 4.78 (s, 2H), 6.60 (dd, J=8.8 Hz, 2.8 Hz, 1H), 6.70-6.75 (m, 2H), 6.91 (dd, J=12.8 Hz, 7.6 Hz, 2H), 7.23-7.34 (m, 2H), 7.39-7.45 (m, 2H), 7.54-7.61 (m, 3H), 8.92 (s, 1H), 9.04 (s, 1H), 10.74 (s, 1H).

### Example 96

### 2-(2-Cyanophenyl)-4-(2-hydroxyphenyl)-6-(2-pyridyl)-4,5-dihydro-1,2,4-triazin-3(2H)-one

¹H-NMR (400 MHz, CDCl₃); δ (ppm) 3.87 (s, 2H), 6.69-6.82 (m, 1H), 6.94-7.10 (m, 2H), 7.22-7.52 (m, 5H), 7.62-7.66 (m, 1H), 7.72-7.76 (m, 1H), 7.98-8.05 (m, 1H), 8.54-8.67 (m, 1H).

ESI-Mass; 370 [M⁺+H]

The title compounds of Examples 97 to 103 were synthesized according to the method described in the above-mentioned Example 87.

### Example 97

### 2-(2-Cyanophenyl)-6-(2-methoxyphenyl)-4-phenyl-4,5-dihydro-1,2,4-triazin-3(2H)-one

¹H-NMR (400 MHz, CDCl₃); δ (ppm) 3.87 (s, 3H), 4.86 (s, 2H), 6.93 (d, J=8.4 Hz, 1H), 7.04 (td, J=7.2 Hz, 0.8 Hz, 1H), 7.20-7.27 (m, 1H), 7.34-7.45 (m, 6H), 7.65 (td, J=7.8 Hz, 1.6 Hz, 1H), 7.71 (dd, J=7.6 Hz, 1.6 Hz, 1H), 7.77-7.81 (m, 2H).

ESI-Mass; 383 [M⁺+H]

### Example 98

### 4-(2-Cyanophenyl)-6-(2-methoxyphenyl)-2-phenyl-4,5-dihydro-1,2,4-triazin-3(2H)-one

¹H-NMR (400 MHz, CDCl₃); δ (ppm) 3.85 (s, 3H), 4.85 (s, 2H), 6.90-6.94 (m, 1H), 6.99-7.06 (m, 1H), 7.20-7.26 (m, 1H), 7.33-7.43 (m, 4H), 7.49-7.52 (m, 1H), 7.62-7.77 (m, 5H).

ESI-Mass; 383 [M⁺+H]

### Example 99

### 2-(2-Cyanophenyl)-6-(2-methoxyphenyl)-4-(2-pyridyl)-4,5-dihydro-1,2,4-triazin-3(2H)-one

¹H-NMR (400 MHz, CDCl₃); δ (ppm) 3.91 (s, 3H), 5.21 (s, 2H), 6.95 (d, J=8.4 Hz, 1H), 7.00-7.08 (m, 2H), 7.39-7.44 (m, 2H), 7.64-7.80 (m, 5H), 7.98-8.01 (m, 1H), 8.39 (ddd, J=4.8 Hz, 1.8 Hz, 0.8 Hz, 1H)

ESI-Mass; 384 [M⁺+H]

### Example 100

### 2-(2-Cyanophenyl)-4-phenyl-6-(2-pyridyl)-4,5-dihydro-1,2,4-triazin-3(2H)-one

¹H-NMR (400 MHz, CDCl₃); δ (ppm) 5.17 (s, 2H), 7.24-7.33 (m, 2H), 7.38-7.44 (m, 3H), 7.46-7.50 (m, 2H), 7.65-7.69 (m, 1H), 7.74-7.79 (m, 3H), 8.19-8.22 (m, 1H), 8.57 (ddd, J=4.8 Hz, 1.6Hz, 0.8 Hz, 1H).

ESI-Mass; 354 [M⁺+H]

### Example 101

### 2-(2-Cyanophenyl)-6-(2-pyridyl)-4-(thiophene-3-yl)-4,5-dihydro-1,2,4-triazin-3(2H)-one

¹H-NMR (400 MHz, CDCl₃); δ (ppm) 5.19 (s, 2H), 7.28-7.35 (m, 3H), 7.43 (td, J=7.6 Hz, 1.2 Hz, 1H), 7.47 (dd, J=5.2 Hz, 1.6 Hz, 1H), 7.66-7.79 (m, 4H), 8.19 (dt, J=8.0 Hz, 1.0 Hz, 1H), 8.59-8.61 (m, 1H).

### Example 102

### 2-(2-Cyanophenyl)-6-(2-pyridyl)-4-(2-pyridyl)-4,5-dihydro-1,2,4-triazin-3(2H)-one

¹H-NMR (400 MHz, CDCl₃); δ (ppm) 5.47 (s, 2H), 7.10 (ddd, J=7.0 Hz, 4.6 Hz, 1.0 Hz, 1H), 7.30-7.34 (m, 1H), 7.38-7.52 (m, 2H), 7.64-7.79 (m, 4H), 7.99-8.01 (m, 1H), 8.18 (dd, J=8.0 Hz, 0.8 Hz, 1H), 8.47-8.49 (m, 1H), 8.64-8.66 (m, 1H).

ESI-Mass; 355 [M⁺+H]

### Example 103

### 2-(2-Cyanophenyl)-6-(2-pyridyl)-4-(3-pyridyl)-4,5-dihydro-1,2,4-triazin-3(2H)-one

¹H-NMR (400 MHz, CDCl₃); δ (ppm) 5.22 (s, 2H), 7.32-7.38 (m, 2H), 7.44 (td, J=7.6 Hz, 1.2 Hz, 1H), 7.70 (td, J=7.4 Hz, 1.6 Hz, 1H), 7.74-7.80 (m, 3H), 7.88 (ddd, J=8.4 Hz, 2.8 Hz, 1.2 Hz, 1H), 8.19 (d, J=8.0 Hz, 1H), 8.50-8.53 (m, 1H), 8.59 (ddd, J=4.8 Hz, 1.6 Hz, 0.8 Hz, 1H), 8.78-8.82(m, 1H).

ESI-Mass; 355 [M⁺+H]

The title compounds of Examples 104 to 111 were synthesized according to the method described in the above-mentioned Example 88.

### Example 104

### 4-(2-Cyanophenyl)-2-phenyl-6-(2-pyridyl)-4,5-dihydro-1,2,4-triazin-3(2H)-one

¹H-NMR (400 MHz, CDCl₃); δ (ppm) 5.15 (s, 2H), 7.21-7.33 (m, 2H), 7.36-7.46 (m, 3H), 7.52-7.57 (m, 1H), 7.65-7.79 (m, 5H), 8.18 (d, J=8.4 Hz, 1H), 8.54-8.56 (m, 1H).

ESI-Mass; 354 [M⁺+H]

### Example 105

### 2-Phenyl-6-(2-pyridyl)-4-(thiophen-3-yl)-4,5-dihydro-1,2,4-triazin-3(2H)-one

¹H-NMR (400 MHz, CDCl₃); δ (ppm) 5.14 (s, 2H), 7.24-7.28 (m, 1H), 7.28-7.34 (m, 3H), 7.42-7.47 (m, 2H), 7.50 (dd, J=5.2 Hz, 1.2 Hz, 1H), 7.52-7.65 (m, 2H), 7.76 (td, J=7.8 H,z 2.0 Hz, 1H), 8.16 (d, J=8.0 Hz, 1H), 8.59-8.61 (m, 1H).

ESI-Mass; 335[M⁺+H]

### Example 106

### 2-(2-Bromophenyl)-6-(2-pyridyl)-4-(thiophen-3-yl)-4,5-dihydro-1,2,4-triazin-3(2H)-one

¹H-NMR (400 MHz, CDCl₃); δ (ppm) 5.18 (brs, 2H), 7.24-7.32 (m, 4H), 7.45 (t, J=7.6 Hz, 1H), 7.52-7.54 (m, 1H), 7.59-7.61 (m, 1H), 7.69-7.74 (m, 2H), 8.08 (dd, J=7.8 Hz, 1.0 Hz, 1H), 8.59-8.61 (m, 1H).

ESI-Mass; 415 [M⁺+H]

### Example 107

### 4-(2,4-Dimethoxyphenyl)-2-phenyl-6-(2-pyridyl)-4,5-dihydro-1,2,4-triazin-3(2H)-one

¹H-NMR (400 MHz, CDCl₃); δ (ppm) 3.82 (s, 3H), 3.83 (s, 3H), 4.96 (s, 2H), 6.49 (dd, J=8.4 Hz, 2.8 Hz, 1H), 6.53 (d, J=2.8 Hz, 1H), 7.20-7.23 (m, 3H), 7.36-7.42 (m, 2H), 7.66-7.67 (m, 2H), 7.75 (td, J=8.0 Hz, 1.6 Hz, 1H), 8.18 (d, J=8.4 Hz, 1H), 8.54 (d, J=4.8 Hz, 1H).

ESI-Mass; 389 [M⁺+H]

### Example 108

### 2-(2-Bromophenyl)-6-(2-methoxyphenyl)-4-(thiophen-3-yl)-4,5-dihydro-1,2,4-triazin-3(2H)-one

¹H-NMR (400 MHz, CDCl₃); δ (ppm) 3.92 (s, 3H), 4.89 (s, 2H), 6.95 (d, J=8.4 Hz, 1H), 7.10 (t, J=7.6 Hz, 1H), 7.14-7.16 (m, 1H), 7.21-7.29 (m, 3H), 7.38-7.62 (m, 3H), 7.62-7.64 (m, 1H), 7.67-7.70 (m, 1H).

### Example 109

### 2-Phenyl-6-(2-pyridyl)-4-(3-pyridyl)-4,5-dihydro-1,2,4-triazin-3(2H)-one

¹H-NMR (400 MHz, CDCl₃); δ (ppm) 5.16 (s, 2H), 7.27-7.36 (m, 3H), 7.45 (t, J=7.8 Hz, 2H), 7.63-7.66 (m, 2H), 7.77 (td, J=8.0 Hz, 1.8 Hz, 1H), 7.86 (ddd, J=8.2 Hz, 2.8 Hz, 1.6 Hz, 1H), 8.19 (dt, J=8.0 Hz, 1.0 Hz, 1H), 8.48 (dd, J=4.8 Hz, 1.6 Hz, 1H), 8.58 (ddd, J=4.8 Hz, 1.8 Hz, 0.8 Hz, 1H), 8.78 (d, J=2.0 Hz, 1H).

ESI-Mass; 330 [M⁺+H]

### Example 110

### 2-(2-Bromophenyl)-6-(2-pyridyl)-4-(3-pyridyl)-4,5-dihydro-1,2,4-triazin-3(2H)-one

¹H-NMR (400 MHz, CDCl₃); δ (ppm) 5.20 (brs, 2H), 7.26-7.35 (m, 2H), 7.43-7.48 (m, 2H), 7.60-7.76 (m, 3H), 7.89 (ddd, J=7.8 Hz, 2.8 Hz, 1.4 Hz, 1H), 8.10 (d, J=8.0 Hz, 1H), 8.48 (dd, J=4.8 Hz, 1.6 Hz, 1H), 8.59 (d, J=4.8 Hz, 1H), 8.80 (d, J=2.4 Hz, 1H).

ESI-Mass; 410 [M⁺+H]

### Example 111

### 2-(2-Bromophenyl)-4-(2-cyanophenyl)-6-(2-pyridyl)-4,5-dihydro-1,2,4-triazin-3(2H)-one

¹H-NMR (400 MHz, CDCl₃); δ (ppm) 5.19 (s, 2H), 7.24-7.32 (m, 2H), 7.41 (dd, J=7.6 Hz, 1.2 Hz, 1H), 7.45 (dd, J=7.6 Hz, 1.2 Hz, 1H), 7.57 (dd, J=8.0 Hz, 0.8 Hz, 1H), 7.64-7.75 (m, 5H), 8.11 (d, J=8.0 Hz, 1H), 8.55 (ddd, J=5.0 Hz, 1.8 Hz, 0.8 Hz, 1H).

The title compounds of Examples 112 to 117 were synthesized according to the method described in the above-mentioned Example 89.

### Example 112

### 2-(2-Bromophenyl)-4,6-diphenyl-4,5-dihydro-1,2,4-triazin-3(2H)-one

¹H-NMR (400 MHz, CDCl₃); δ (ppm) 4.92 (s, 2H), 7.22-7.27 (m, 2H), 7.39-7.47 (m, 8H), 7.62-7.65 (m, 1H), 7.67-7.70 (m, 1H), 7.72-7.75 (m, 2H).

### Example 113

### 4-(2-Bromophenyl)-2,6-diphenyl-4,5-dihydro-1,2,4-triazin-3(2H)-one

¹H-NMR (400 MHz, CDCl₃); δ (ppm) 4.67 (d, J=15.4 Hz, 1H), 4.94 (d, J=15.4 Hz, 1H), 7.21-7.31 (m, 3H), 7.36-7.46 (m, 6H), 7.49 (dd, J=8.0Hz, 1.6Hz, 1H), 7.67-7.71 (m, 2H), 7.73-7.77 (m, 2H).

ESI-Mass; 406 [M⁺+H]

### Example 114

### 2-(2-Bromophenyl)-4-(2-bromophenyl)-6-phenyl-4,5-dihydro-1,2,4-triazin-3(2H)-one

¹H-NMR (400 MHz, CDCl₃); δ (ppm) 4.74(d, J=15.6 Hz, 1H), 5.44 (d, J=15.6 Hz, 1H), 6.78-6.82 (m, 1H), 6.95-6.98 (m, 1H), 7.12-7.30 (m, 3H), 7.38-7.53 (m, 4H), 7.59-7.74 (m, 3H), 8.54-8.58 (m, 1H).

ESI-Mass; 486[M⁺+H]

### Example 115

### 4-(2-Bromophenyl)-6-(2-methoxyphenyl)-2-phenyl-4,5-dihydro-1,2,4-triazin-3(2H)-one

¹H-NMR (400 MHz, CDCl₃); δ (ppm) 3.82 (s, 3H), 4.55-4.82 (m, 2H), 6.88-6.98 (m, 1H), 7.04 (t, J=7.6 Hz, 1H), 7.13-7.27 (m, 3H), 7.32-7.41 (m, 3H), 7.45 (td, J=7.8 Hz, 1.6 Hz, 1H), 7.61-7.71 (m, 3H), 7.76 (dd, J=7.6 Hz, 1.6 Hz, 1H).

ESI-Mass; 436[M⁺+H]

### Example 116

### 2-(2-Bromophenyl)-4-(2,5-dimethoxyphenyl)-6-(2-methoxyphenyl)-4,5-dihydro-1,2,4-tria zin-3(2H)-one

¹H-NMR (400 MHz, CDCl₃); δ (ppm) 3.75 (s, 6H), 3.82 (s, 3H), 4.70 (s, 2H), 6.80 (dd, J=9.0 Hz, 3.0 Hz, 1H), 6.87 (d, J=8.8 Hz, 1H), 6.89 (d, J=8.4 Hz, 1H), 6.96 (d, J=2.8 Hz, 1H), 7.00 (td, J=7.4 Hz, 0.4 Hz, 1H), 7.17-7.20 (m, 1H), 7.35-7.40 (m, 2H), 7.64 (dd, J=4.8 Hz, 1.6 Hz, 1H), 7.66 (dd, J=4.8 Hz, 1.6 Hz, 1H), 7.73 (dd, J=7.6 Hz, 1.6 Hz, 1H).

### Example 117

### 4-(2,5-Dimethoxyphenyl)-6-(2-methoxyphenyl)-2-phenyl-4,5-dihydro-1,2,4-triazin-3(2H)-one

¹H-NMR (400 MHz, CDCl₃); δ (ppm) 3.74 (s, 3H), 3.76 (s, 3H), 3.81 (s, 3H), 4.66 (s, 2H), 6.83 (dd, J=9.0 Hz, 3.0 Hz, 1H), 6.86-6.94 (m, 3H), 7.03 (t, J=7.6 Hz, 1H), 7.17 (t, J=7.4 Hz, 1H), 7.32-7.41 (m, 3H), 7.67-7.71 (m, 2H), 7.75 (dd, J=7.6 Hz, 1.6 Hz, 1H).

ESI-Mass; 418 [M⁺+H]

### Example 118

### 2-(2-Bromophenyl)-6-(2-pyridyl)-4-(2-pyridyl)-4,5-dihydro-1,2,4-triazin-3(2H)-one

The title compound was synthesized according to the method described in Example 92 above.

¹H-NMR (400 MHz, CDCl₃); δ (ppm) 5.16 (m, 1H), 5.25-5.51 (m, 1H), 7.06-7.09 (m, 1H), 7.27-7.32 (m, 1H), 7.37-7.52 (m, 2H), 7.59-7.74 (m, 4H), 8.03 (d, J=8.4 Hz, 1H), 8.08-8.11 (m, 1H), 8.46-8.49 (m, 1H), 8.63-8.66 (m, 1H).

ESI-Mass; 408[M⁺+H]

### Example 119

### 2-Phenyl-4-phenyl-6-(2-pyrimidyl)-4,5-dihydro-1,2,4-triazin-3(2H)-one

### 119-1) N-Methoxycarbonyl-N-phenylglycine

N-Phenylglycine (7.2 g) was dissolved in t-butyl methyl ether (120 ml), and 1 N aqueous sodium hydroxide (105 ml) was added thereto. The mixture was cooled to 0° C., and methyl chlorocarbonate (6 ml) was added dropwise thereto under vigorous stirring and then stirred at room temperature overnight. The organic layer was removed, and an aqueous saturated sodium dihydrogen phosphate solution was added to the aqueous layer, followed by extracting with ethyl acetate. The organic layer was washed with water and dried over anhydrous sodium sulfate. After the drying agent was filtered off, the filtrate was evaporated. The residue was purified by silica gel column chromatography (hexane-ethyl acetate system), to give the title compound (9.1 g, 92%) as colorless crystals.

¹H-NMR (400 MHz, CDCl₃); δ (ppm) 3.27 (brs, 3H), 4.39 (m, 2H), 7.24-7.39 (m, 5H).

### 119-2) N-Methoxycarbonyl-N-phenylamino-2-ethanol

N-Methoxycarbonyl-N-phenylglycine (395 mg) was dissolved in tetrahydrofuran anhydride (50 ml), cooled to 0° C., and 1.0 M borane-tetrahydrofuran complex in tetrahydrofuran (2.4 ml) was added dropwise in a nitrogen atmosphere. After stirring at 0° C. for 2 hours, 1.0 M borane-tetrahydrofuran complex in tetrahydrofuran (2.4 ml) was further added dropwise. This procedure was repeated twice. After stirring at 0° C. for 4 hours, methanol (40 ml) was added dropwise, and after stirring at the same temperature for 2 minutes, the mixture was evaporated. Ethyl acetate was added thereto, and the reaction solution was washed with an aqueous saturated sodium bicarbonate solution and dried over anhydrous sodium sulfate. After the drying agent was filtered off, the filtrate was evaporated, to give the title compound as a colorless oil (420 mg, quantitative).

¹H-NMR (400 MHz, CDCl₃); δ (ppm) 3.68 (brs, 3H), 3.76 (t, 2H), 3.83 (dt, 2H), 7.15-7.39 (m, 5H).

### 119-3) N-Methoxycarbonyl-N-phenyl-aminoacetaldehyde

N-Methoxycarbonyl-N-phenylamino-2-ethanol (420 mg) was dissolved in dimethyl sulfoxide (13 ml), and triethylamine (5 ml) was added thereto, followed by cooling to 0° C. Sulfur trioxide (500 mg) was added little by little thereto under stirring vigorously at the same temperature, followed by stirring at room temperature overnight. Water was added thereto, and the reaction solution was extracted with ethyl acetate. The organic layer was washed with an aqueous saturated ammonium chloride solution and an aqueous saturated sodium hydroxide solution, and dried over anhydrous sodium sulfate. After the drying agent was filtered off, the filtrate was evaporated. The residue was purified by silica gel column chromatography (hexane-ethyl acetate system), to give the title compound as a brown oil (191 mg, 46%).

¹H-NMR (400 MHz, CDCl₃); δ (ppm) 3.72 (s, 3H), 4.40 (s, 2H), 7.24-7.39 (m, 5H), 9.70 (s, 1H).

### 119-4) N-Phenyl-2-(N″-phenyl-N″-methoxycarbonylamino)ethanehydrazonoyl bromide

N-Methoxycarbonyl-N-phenylaminoacetaldehyde (500 mg) was dissolved in ethanol (20 ml), and phenylhydrazine (280 mg) was added thereto, and the mixture was stirred overnight in a nitrogen atmosphere. The reaction solution was evaporated, and from N-methoxycarbonyl-N-phenylaminoacetaldehyde phenylhydrazone obtained as the residue, the title compound (158 mg) was obtained as a reddish brown oil, according to the method in Tetrahedron Vol. 52, pp. 661-668, 1996.

¹H-NMR (400 MHz, CDCl₃); δ (ppm) 3.75 (s, 3H), 4.79 (s, 2H), 6.85 (d, 2H), 7.22-7.38 (m, 8H), 7.68 (s, 1H).

### 119-5) (Z)-2′-(N-Phenyl-N-methoxycarbonylamino)-2-acetylpyrimidine phenylhydrazone

N-Phenyl-2-(N″-phenyl-N″-methoxycarbonylamino)ethane hydrazonoyl bromide (158 mg) was dissolved in xylene (10 ml), and 2-trinormalbutyl stannyl pyrimidine (241 mg), tetrakistriphenyl phosphine palladium (25 mg) and copper iodide (5 mg) were added thereto, followed by stirring at 110° C. for 4 hours in a nitrogen atmosphere. After cooling to room temperature, the mixture was extracted with ethyl acetate. The organic layer was washed with water and dried over anhydrous sodium sulfate. After the drying agent was filtered off, the filtrate was evaporated. The residue was purified by silica gel column chromatography (hexane-ethyl acetate system), to give the title compound (37 mg, 23%) as brown crystals.

¹H-NMR (400 MHz, CDCl₃); δ (ppm) 3.73 (s, 3H), 5.10 (s, 2H), 7.01-7.11 (m, 2H), 7.15-7.20 (m, 2H), 7.25-7.30 (m, 3H), 7.35-7.39 (m, 4H), 8.81 (d, 2H), 13.30 (s, 1H).

### 119-6) (E)-2′-(N-phenyl-N-methoxycarbonylamino)-2-acetylpyridine phenylhydrazone

(Z)-2′-(N-Phenyl-N-methoxycarbonylamino)-2-acetylpyrimidine phenylhydrazone (5 mg) was dissolved in 4 N hydrochloric acid-ethyl acetate (0.5 ml) at 0° C. After stirring at room temperature for 2 minutes, the reaction solution was neutralized by adding an aqueous saturated sodium bicarbonate solution. The reaction solution was extracted with ethyl acetate, and the organic layer was washed with water and dried over anhydrous sodium sulfate. After the drying agent was filtered off, the filtrate was evaporated. The residue was purified by silica gel column chromatography (hexane-ethyl acetate system), to give the title compound (5 mg) as brown crystals.

¹H-NMR (400 MHz, CDCl₃); δ (ppm) 3.74 (s, 3H), 5.28 (s, 2H), 6.95 (t, 1H), 7.06-7.08(m, 2H), 7.16-7.21(m, 3H), 7.26-7.30(m, 3H), 7.41-7.44(m, 2H), 8.49(d, 2H), 10.45(s, 1H).

### 119-7) 2-Phenyl-4-phenyl-6-(2-pyrim idyl)-4,5-dihydro-1,2,4-triazin-3(2H)-one

(E)-2′-(N-Phenyl-N-methoxycarbonylamino)-2-acetylpyrimidine phenylhydrazone (5 mg) was dissolved in ethanol (3 ml), and sodium ethylate (1.1 mg) was added thereto at 0° C., followed by stirring at room temperature for 1 hour. After heating at 110° C. for 1 minute, it was cooled to room temperature. An aqueous saturated ammonium chloride solution and water were added thereto, and the reaction solution was extracted with ethyl acetate. The organic layer was washed with water and dried over anhydrous sodium sulfate. After the drying agent was filtered off, the filtrate was evaporated. The residue was purified by silica gel column chromatography (hexane-ethyl acetate system), to give the title compound (2 mg) as colorless crystals.

¹H-NMR (400 MHz, CDCl₃); δ (ppm) 5.12(s, 2H), 7.25-7.30(m, 2H), 7.32(t, 1H), 7.40-7.47(m, 4H), 7.51-7.57(m, 4H), 8.85(d, 2H).

The title compounds of Examples 120 to 126 were synthesized according to the above-mentioned Example 85.

### Example 120

### 2-(2-Bromophenyl)-4-(4-biphenyl)-6-(2-pyridyl)-4,5-dihydro-1,2,4-triazin-3(2H)-one

¹H-NMR (400 MHz, CDCl₃); δ (ppm) 5.20 (brs, 2H), 7.19-7.75 (m, 15H), 8.11 (d, 1H), 8.57-8.59 (m, 1H).

### Example 121

### 2-(2-Bromophenyl)-4-(3-nitrophenyl)-6-(2-pyridyl)-4,5-dihydro-1,2,4-triazin-3(2H)-one

¹H-NMR (400 MHz, CDCl₃); δ (ppm) 5.23(brs, 2H), 7.22-7.35 (m,2H), 7.47 (td, 1H), 7.55 (t, 1H), 7.60 (dd, 1H), 7.70-7.78 (m, 2H), 7.91 (dd, 1H), 8.08-8.12 (m, 2H), 8.38 (t, 1H), 8.60 (m, 1H).

ESI-Mass; 452[M⁺+H]

### Example 122

### 2-(2-Bromophenyl)-4-(4-fluorophenyl)-6-(2-pyridyl)-4,5-dihydro-1,2,4-triazin-3(2H)-one

¹H-NMR (400 MHz, CDCl₃); δ (ppm) 5.12 (brs, 2H), 7.06-7.11 (m, 2H), 7.24-7.29 (m, 1H), 7.30 (ddd, 1H), 7.41-7.49 (m, 3H), 7.60 (dd, 1H), 7.68-7.75 (m, 2H), 8.10 (d, 1H), 8.56-8.58 (m, 1H).

### Example 123

### 2-(2-Bromophenyl)-4-(3-formylphenyl)-6-(2-pyridyl)-4,5-dihydro-1,2,4-triazin-3(2H)-one

¹H-NMR (400 MHz, CDCl₃); δ (ppm) 5.20 (brs, 2H), 7.18-7.34 (m, 2H), 7.46 (td, 1H), 7.57 (t, 1H), 7.63 (dd, 1H), 7.56-7.70 (m, 3H), 7.83 (ddd, 1H), 8.02 (t, 1H), 8.11 (dt, 1H), 8.58-8.59 (m, 1H), 10.03 (s, 1H).

### Example 124

### 2-(2-Bromophenyl)-4-(3-tolyl)-6-(2-pyridyl)-4,5-dihydro-1,2,4-triazin-3(2H)-one

¹H-NMR (400 MHz, CDCl₃); δ (ppm) 2.37 (s, 3H), 5.13 (brs, 2H), 7.04 (t, 1H), 7.23-7.33 (m, 5H), 7.44 (td, 1H), 7.62 (dd, 1H), 7.68-7.70(m, 1H), 7.73(dd, 1H), 8.10 (d, 1H), 8.56-8.58 (m, 1H).

### Example 125

### 2-(2-Bromophenyl)-4-(4-thiomethoxyphenyl)-6-(2-pyridyl)-4,5-dihydro-1,2,4-triazin-3(2H)-one

¹H-NMR (400 MHz, CDCl₃); 6 (ppm) 2.50 (s, 3H), 5.13 (brs, 2H), 7.26-7.32 (m, 3H), 7.42-7.47 (m, 2H), 7.53-7.57 (m, 1H), 7.63 (dd, 1H), 7.70-7.76 (m, 3H), 8.11 (d, 1H), 8.57-8.60 (m, 1H).

### Example 126

### 2-(2-Bromophenyl)-4-(2-chloropyridin-5-yl)-6-(2-pyridyl)-4,5-dihydro-1,2,4-triazin-3(2H)-one

¹H-NMR (400 MHz, CDCl₃); δ (ppm) 5.18 (brs, 2H), 6.99-8.11 (m, 10H), 8.56-8.60 (m, 1H).

### Example 127

### 2-(2-Cyanophenyl)-4-(3-nitrophenyl)-6-(2-pyridyl)-4,5-dihydro-1,2,4-triazin-3(2H)-one

According to the above-mentioned Example 87, the title compound was synthesized from 2-(2-bromophenyl)-4-(3-nitrophenyl)-6-(2-pyridyl)-4,5-dihydro-1,2,4-triazin-3(2H)-one.

¹H-NMR (400 MHz, CDCl₃); δ (ppm) 5.2 4 (brs, 2H), 7.36 (ddd, 1H), 7.46 (td, 1H), 7.59 (t, 1H), 7.69-7.81 (m, 4H), 7.90 (ddd, 1H), 8.12 (ddd, 1H), 8.20 (dt, 1H), 8.38 (t, 1H), 8.60 (ddd, 1H).

ESI-Mass; 399[M⁺+H]

### Example 128

### 2-(2-Cyanophenyl)-4-(3-aminophenyl)-6-(2-pyridyl)-4,5-dihydro-1,2,4-triazin-3(2H)-one

2-(2-Cyanophenyl)-4-(3-nitrophenyl)-6-(2-pyridyl)-4,5-dihydro-1,2,4-triazin-3(2H)-one (15 mg) was dissolved in methanol (3 ml), 10% palladium-carbon powder (hydrate) (21 mg) was added thereto, and the mixture was stirred for 4 hours at room temperature in a hydrogen atmosphere. The palladium-carbon powder was filtered off, and the filtrate was evaporated. The resulting residue was purified by silica gel column chromatography (hexane-ethyl acetate system), to give the title compound (13 mg) as colorless crystals.

¹H-NMR (400 MHz, CDCl₃); δ (ppm) 3.72 (brs, 2H), 5.13 (s, 2H), 6.58 (ddd, 1H), 6.82-6.85 (m, 2H), 7.19 (t, 1H), 7.32 (ddd, 1H), 7.40 (td, 1H), 7.67 (td, 1H), 7.73-7.78 (m, 3H), 8.20 (dt, 1H), 8.58 (ddd, 1H).

ESI-Mass; 369 [M⁺+H]

### Example 129

### 2-(2-Chlorophenyl)-4-phenyl-6-(2-pyrimidyl)-4,5-dihydro-1,2,4-triazin-3(2H)-one

The title compound was synthesized according to the above-mentioned Example 119.

¹H-NMR (400 MHz, CDCl₃); δ (ppm) 5.18(brs, 2H), 7.25(tt, 1H), 7.28-7.44(m, 5H), 7.47-7.51(m, 3H), 7.65(dd, 1H), 8.84(d, 2H).

ESI-Mass; 364 [M⁺+H]

The chemical structures of the compounds of the above-mentioned Examples are shown below. Each symbol in the table corresponds to the symbol for each substituent group in the structural formula shown in the title of the table. Each substituent group is bound directly via a single bond having a substituent-free terminal, as shown in the structural formula in the table. “Me” in the table means a methyl group.

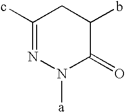

| Example | a | b | c |
|---|---|---|---|
| 1 | 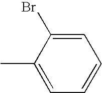 | 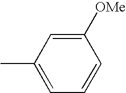 | 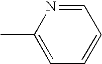 |
| 2 | 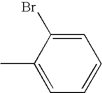 | 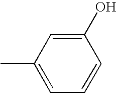 | 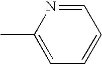 |
| 14 | 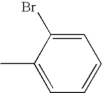 | 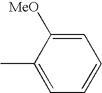 | 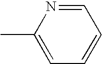 |
| 15 | 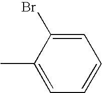 | 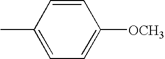 | 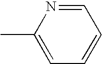 |
| 16 | 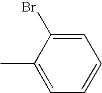 | 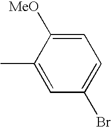 | 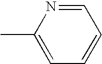 |
| 17 | 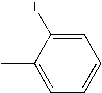 | 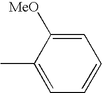 | 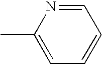 |
| 18 | 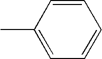 | 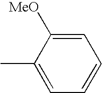 | 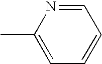 |
| 19 | 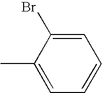 | 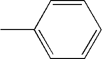 | 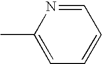 |
| 20 | 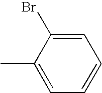 | 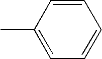 | 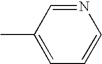 |
| 21 | 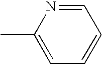 | 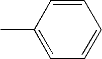 | 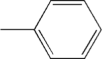 |
| 22 | 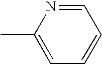 | 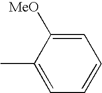 | 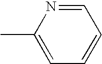 |
| 24 | 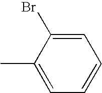 | 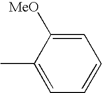 | 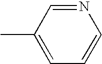 |
| 25 | 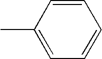 | 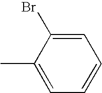 | 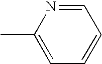 |
| 26 | 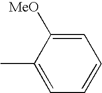 | 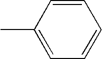 | 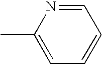 |
| 27 | 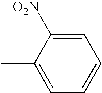 | 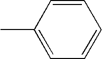 | 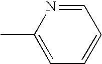 |
| 28 | 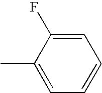 | 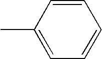 | 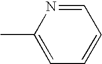 |
| 29 | 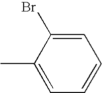 | 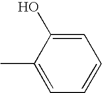 | 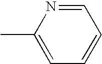 |
| 30 | 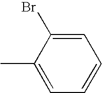 | 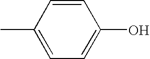 | 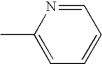 |
| 33 | 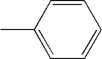 | 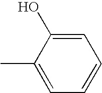 | 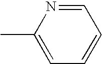 |
| 34 | 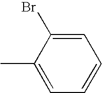 | 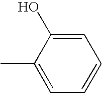 | 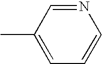 |
| 38 | 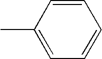 | 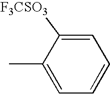 | 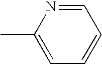 |
| 39 | 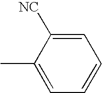 | 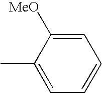 | 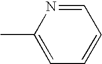 |
| 41 | 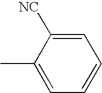 | 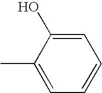 | 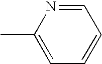 |
| 76 | 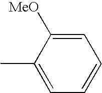 | 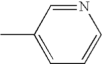 | 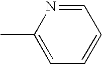 |

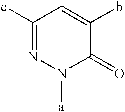

| Example | a | b | c |
|---|---|---|---|
| 3 | 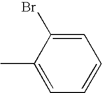 | 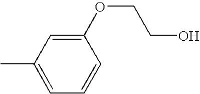 | 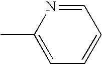 |
| 4 | 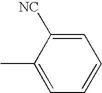 | 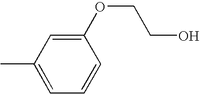 | 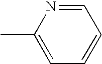 |
| 5 | 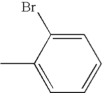 | 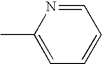 | 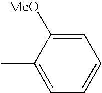 |
| 23 | 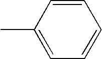 | 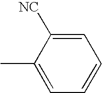 | 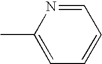 |
| 31 | 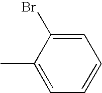 | 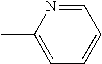 | 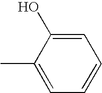 |
| 32 | 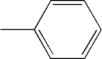 | 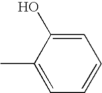 | 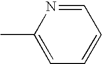 |
| 35 | 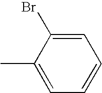 | 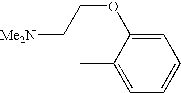 | 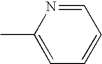 |
| 36 | 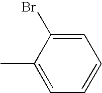 | 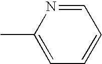 | 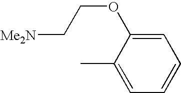 |
| 37 | 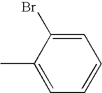 | 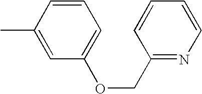 | 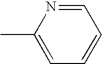 |
| 40 | 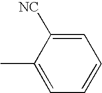 | 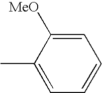 | 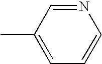 |
| 42 | 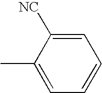 | 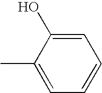 | 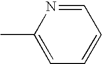 |
| 43 | 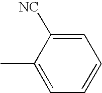 | 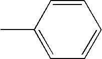 | 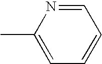 |
| 44 | 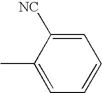 | 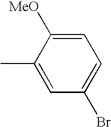 | 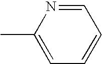 |
| 45 | 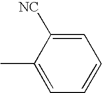 | 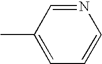 | 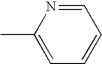 |
| 46 | 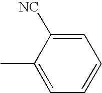 | 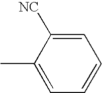 | 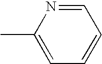 |
| 47 | 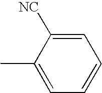 | 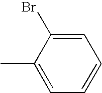 | 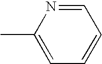 |
| 51 | 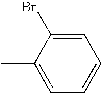 | 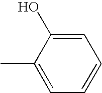 | 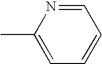 |
| 52 | 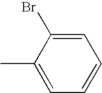 | 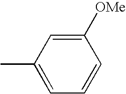 | 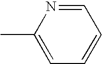 |
| 55 | 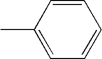 | 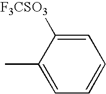 | 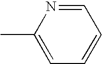 |
| 56 | 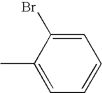 | 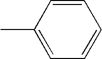 | 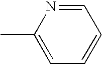 |
| 59 | 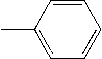 | 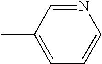 | 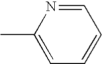 |
| 60 | 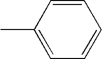 | 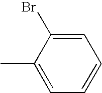 | 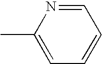 |
| 61 | 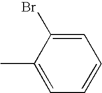 | 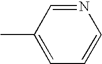 | 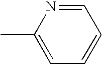 |
| 63 | 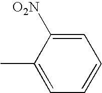 | 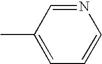 | 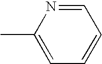 |
| 64 | 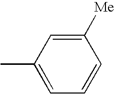 | 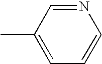 | 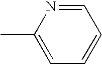 |
| 65 | 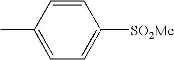 | 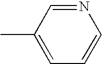 | 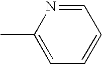 |
| 66 | 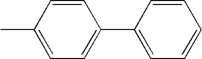 | 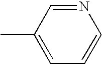 | 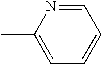 |
| 67 | 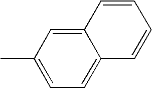 | 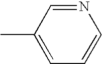 | 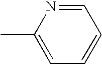 |
| 68 | 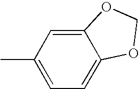 | 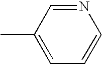 | 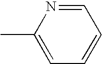 |
| 69 | 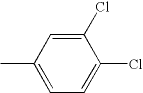 | 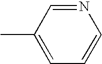 | 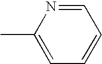 |
| 70 | 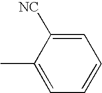 | 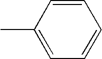 | 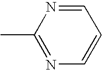 |
| 71 | 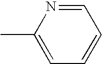 | 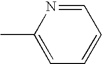 | 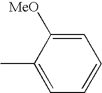 |
| 72 | 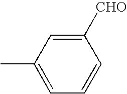 | 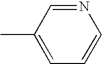 | 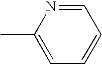 |
| 73 | 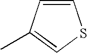 | 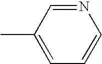 | 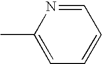 |
| 74 | 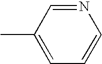 | 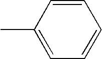 | 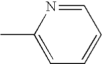 |
| 75 | 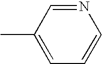 | 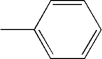 | 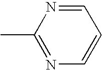 |
| 79 | 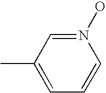 | 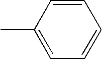 | 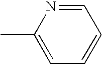 |
| 80A | 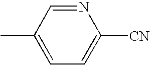 | 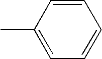 | 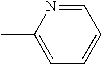 |
| 80B | 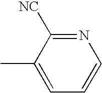 | 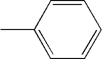 | 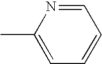 |
| 81A | 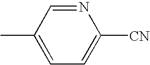 | 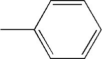 | 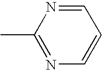 |
| 81B | 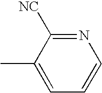 | 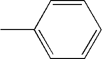 | 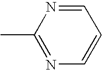 |
| 82 | 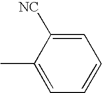 | 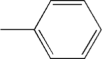 | 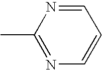 |
| 83 | 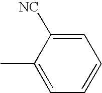 | 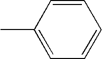 | 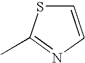 |

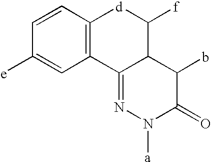

| Example | a | b | d | e | f |
|---|---|---|---|---|---|
| 6 | 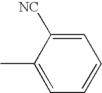 | 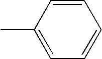 | O | H | H |
| 8 | 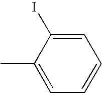 | 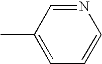 | O | H | H |
| 48 | 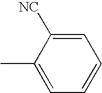 | 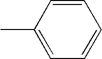 | O | F | H |
| 49 | 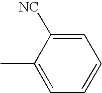 | 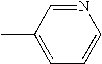 | O | H | H |
| 50 | 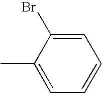 | 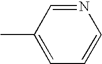 | O | H | H |

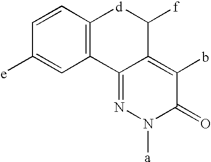

| Example | a | b | d | e | f |
|---|---|---|---|---|---|
| 7 | 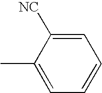 | 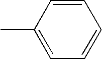 | O | H | H |
| 9 | 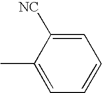 | 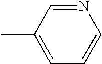 | O | H | H |
| 53 | 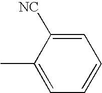 | 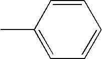 | O | F | OH |
| 54 | 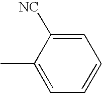 | 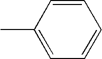 | O | F | H |
| 57 | 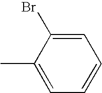 | 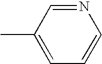 | O | H | H |
| 58 | 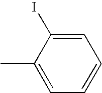 | 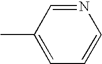 | O | H | H |

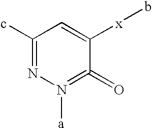

| Example | a | b | c | x |
|---|---|---|---|---|
| 10 | 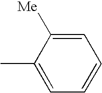 | 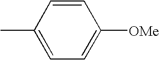 | 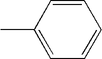 | —CH₂— |
| 12 | 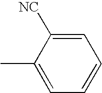 | 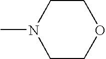 | 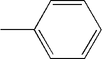 | 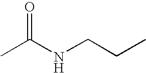 |
| 62 | 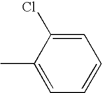 | 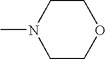 | 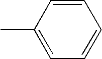 | 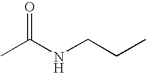 |

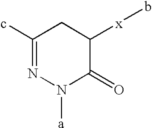

| Example | a | b | c | x |
|---|---|---|---|---|
| 11 | 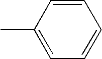 | 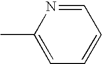 | 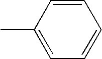 | 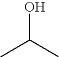 |

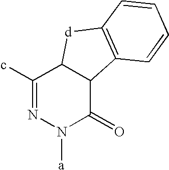

| Example | a | c | d |
|---|---|---|---|
| 13 | 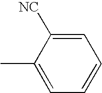 | 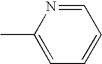 | O |

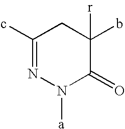

| Example | a | b | c | r |
|---|---|---|---|---|
| 77 | 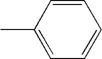 | 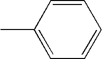 | 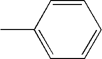 | Me |
| 78 | 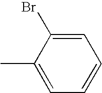 | 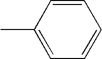 | 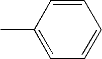 | Me |
| 84 | 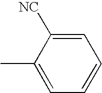 | 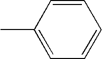 | 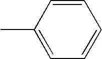 | Me |

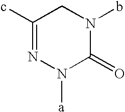

| Example | a | b | c |
|---|---|---|---|
| 85 | 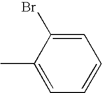 | 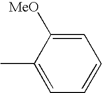 | 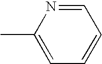 |
| 86 | 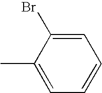 | 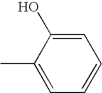 | 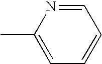 |
| 87 | 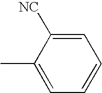 | 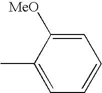 | 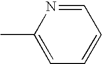 |
| 88 | 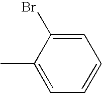 | 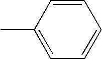 | 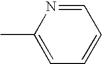 |
| 89 | 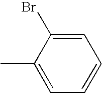 | 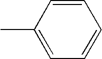 | 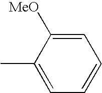 |
| 90 | 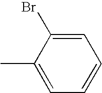 | 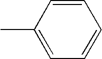 | 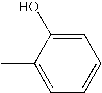 |
| 91 | 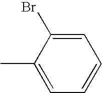 | 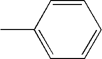 | 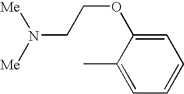 |
| 92 | 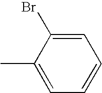 | 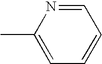 | 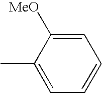 |
| 93 | 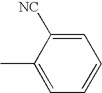 | 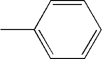 | 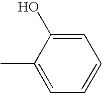 |
| 94 | 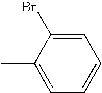 | 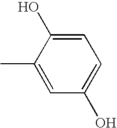 | 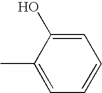 |
| 95 | 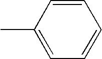 | 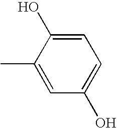 | 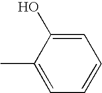 |
| 96 | 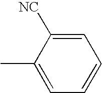 | 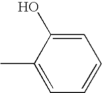 | 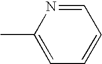 |
| 97 | 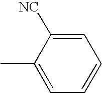 | 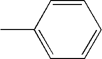 | 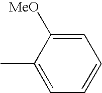 |
| 98 | 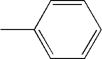 | 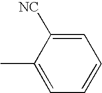 | 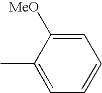 |
| 99 | 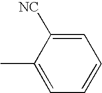 | 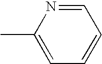 | 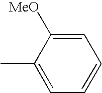 |
| 100 | 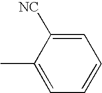 | 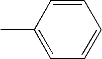 | 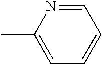 |
| 101 | 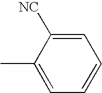 | 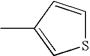 | 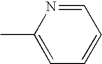 |
| 102 | 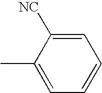 | 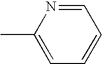 | 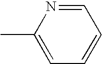 |
| 103 | 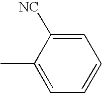 | 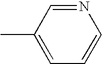 | 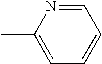 |
| 104 | 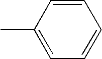 | 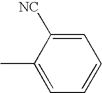 | 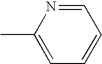 |
| 105 | 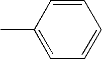 | 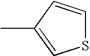 | 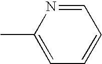 |
| 106 | 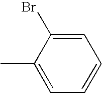 | 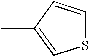 | 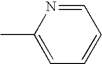 |
| 107 | 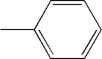 | 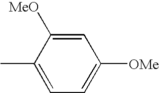 | 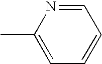 |
| 108 | 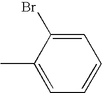 | 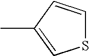 | 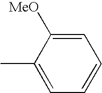 |
| 109 | 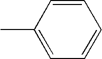 | 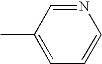 | 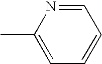 |
| 110 | 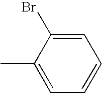 | 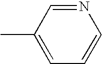 | 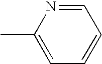 |
| 111 | 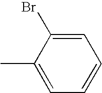 | 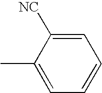 | 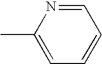 |
| 112 | 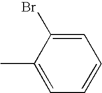 | 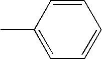 | 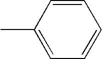 |
| 113 | 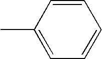 | 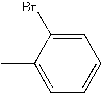 | 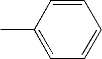 |
| 114 | 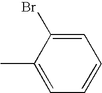 | 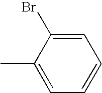 | 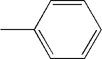 |
| 115 | 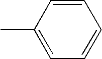 | 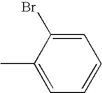 | 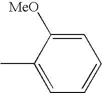 |
| 116 | 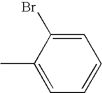 | 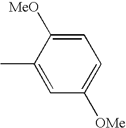 | 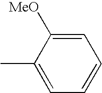 |
| 117 | 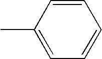 | 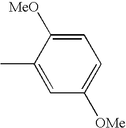 | 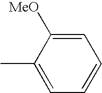 |
| 118 | 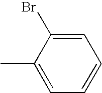 | 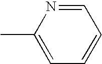 | 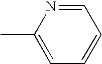 |
| 119 | 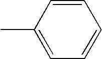 | 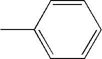 | 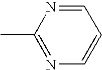 |
| 120 | 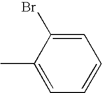 | 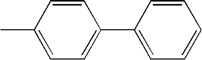 | 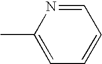 |
| 121 | 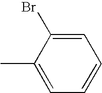 | 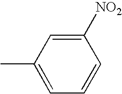 | 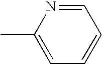 |
| 122 | 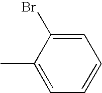 | 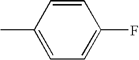 | 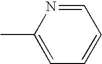 |
| 123 | 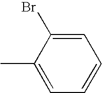 | 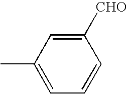 | 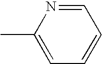 |
| 124 | 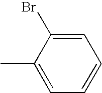 | 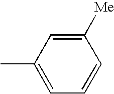 | 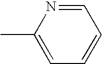 |
| 125 | 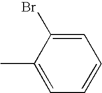 | 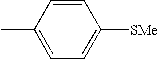 | 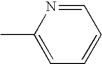 |
| 126 | 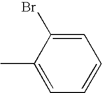 | 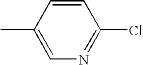 | 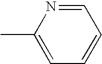 |
| 127 | 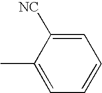 | 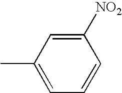 | 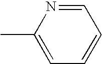 |
| 128 | 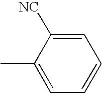 | 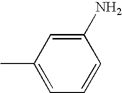 | 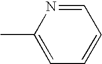 |
| 129 | 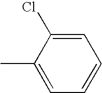 | 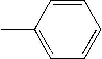 | 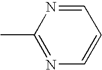 |

Particularly preferable compounds in the above-mentioned Examples include
2-(2-bromophenyl)-4-(3-methoxyphenyl)-6-(2-pyridyl)-4,5-dihydro-3(2H)-pyridazinone,
2-(2-bromophenyl)-4-(3-hydroxyphenyl)-6-(2-pyridyl)-4,5-dihydro-3(2H)-pyridazinone,
2-(2-bromophenyl)-4-[3-(2-hydroxyethoxy)phenyl]-6-(2-pyridyl)-3(2H)-pyridazinone,
2-(2-cyanophenyl)-4-[3-(2-hydroxyethoxy)phenyl]-6-(2-pyridyl)-3(2H)-pyridazinone,
2-(2-bromophenyl)-6-(2-methoxyphenyl)-4-(2-pyridyl)-3 (2H)-pyridazinone,
2-(2-cyanophenyl)-4-phenyl-2,3,4,4a-tetrahydro-5H-(1)benzopyrano[4,3-c]pyridazin-3-on e, 2-(2-cyanophenyl)-4-phenyl-2,3-dihydro-5H-(1)benzopyrano[4,3-c]pyridazin-3-one,
2-(2-iodophenyl)-4-(3-pyridyl)-2,3,4,4a-tetrahydro-5H-(1)benzopyrano[4,3-c]pyridazin-3-one,
2-(2-cyanophenyl)-4-(3-pyridyl)-2,3-dihydro-5H-(1)benzopyrano[4,3-c]pyridazin-3-one,
4-(4-methoxybenzyl)-6-phenyl-2-(2-tolyl)-3(2H)-pyridazinone,
2,6-diphenyl-4-(a-hydroxy-2-picolyl)-4,5-dihydro-3(2H)-pyridazinone,
2-(2-cyanophenyl)-4-(4-morpholinoethylaminocarbonyl)-6-phenyl-3(2H)-pyridazinone,
2-(2-cyanophenyl)-6-(2-pyridyl)-4,5-dihydro-2H-pyridazino[4,5-b]benzofuran-3-one,
2-(2-bromophenyl)-4-(2-methoxyphenyl)-6-(2-pyridyl)-4,5-dihydro-3(2H)-pyridazinone,
2-(2-bromophenyl)-4-(4-methoxyphenyl)-6-(2-pyridyl)-4,5-dihydro-3(2H)-pyridazinone,
2-(2-bromophenyl)-4-(3-bromo-6-methoxyphenyl)-6-(2-pyridyl)-4,5-dihydro-3(2H)-pyridazinone,
2-(2-iodophenyl)-4-(2-methoxyphenyl)-6-(2-pyridyl)-4,5-dihydro-3(2H)-pyridazinone,
4-(2-methoxyphenyl)-2-phenyl-6-(2-pyridyl)-4,5-dihydro-3(2H)-pyridazinone,
2-(2-bromophenyl)-4-phenyl-6-(2-pyridyl)-4,5-dihydro-3(2H)-pyridazinone,
2-(2-bromophenyl)-4-phenyl-6-(3-pyridyl)-4,5-dihydro-3(2H-pyridazinone,
4,6-diphenyl-2-(2-pyridyl)-4,5-dihydro-3(2H)-pyridazinone,
4-(2-methoxyphenyl)-2-(2-pyridyl)-6-(2-pyridyl)-4,5-dihydro-3(2H)-pyridazinone,
4-(2-cyanophenyl)-2-phenyl-6-(2-pyridyl)-3(2H)-pyridazinone,
2-(2-bromophenyl)-4-(2-methoxyphenyl)-6-(3-pyridyl)-4,5-dihydro-3(2H)-pyridazinone,
4-(2-bromophenyl)-2-phenyl-6-(2-pyridyl)-4,5-dihydro-3(2H)-pyridazinone,
2-(2-methoxyphenyl)-4-phenyl-6-(2-pyridyl)-4,5-dihydro-3(2H)-pyridazinone,
4-phenyl-2-(2-nitrophenyl)-6-(2-pyridyl)-4,5-dihydro-3(2H)-pyridazinone,
2-(2-fluorophenyl)-4-phenyl-6-(2-pyridyl)-4,5-dihydro-3(2H)-pyridazinone,
2-(2-bromophenyl)-4-(2-hydroxyphenyl)-6-(2-pyridyl)-4,5-dihydro-3(2H)-pyridazinone,
2-(2-bromophenyl)-4-(4-hydroxyphenyl)-6-(2-pyridyl)-4,5-dihydro-3(2H)-pyridazinone,
2-(2-bromophenyl)-6-(2-hydroxyphenyl)-4-(2-pyridyl)-3(2H)-pyridazinone,
4-(2-hydroxyphenyl)-2-phenyl-6-(2-pyridyl)-3 (2H)-pyridazinone,
4-(2-hydroxyphenyl)-2-phenyl-6-(2-pyridyl)-4,5-dihydro-3(2H)-pyridazinone,
2-(2-bromophenyl)-4-(2-hydroxyphenyl)-6-(3-pyridyl)-4,5-dihydro-3(2H)-pyridazinone,
2-(2-bromophenyl)-4-(2-dimethylaminoethoxyphenyl)-6-(2-pyridyl)-3(2H)-pyridazinone,
2-(2-bromophenyl)-6-(2-dimethylaminoethoxyphenyl)-4-(2-pyridyl)-3(2H)-pyridazinone,
2-(2-bromophenyl)-4-[3-(2-picolyloxyphenyl)]-6-(2-pyridyl)-3(2H)-pyridazinone,
2-phenyl-6-(2-pyridyl)-4-(2-trifluoromethylsulfonyloxyphenyl)-4,5-dihydro-3(2H)-pyrida zinone,
2-(2-cyanophenyl)-4-(2-methoxyphenyl)-6-(2-pyridyl)-4,5-dihydro-3(2H)-pyridazinone,
2-(2-cyanophenyl)-4-(2-methoxyphenyl)-6-(2-pyridyl)-3(2H)-pyridazinone,
2-(2-cyanophenyl)-4-(2-hydroxyphenyl)-6-(2-pyridyl)-4,5-dihydro-3(2H)-pyridazinone,
2-(²-cyanophenyl)-4-(2-hydroxyphenyl)-6-(2-pyridyl)-3(2H)-pyridazinone,
2-(2-cyanophenyl)-4-phenyl-6-(2-pyridyl)-3 (2H)-pyridazinone,
2-(2-cyanophenyl)-4-(3-bromo-6-methoxyphenyl)-6-(2-pyridyl)-3(2H)-pyridazinone,
2-(2-cyanophenyl)-4-(3-pyridyl)-6-(2-pyridyl)-3(2H)-pyridazinone,
2-(2-cyanophenyl)-4-(2-cyanophenyl)-6-(2-pyridyl)-3(2H)-pyridazinone,
4-(2-bromophenyl)-2-(2-cyanophenyl)-6-(2-pyridyl)-3 (2H)-pyridazinone,
2-(2-cyanophenyl)-9-fluoro-4-phenyl-2,3,4,4a-tetrahydro-H-(1)benzopyrano[4,3-c]pyrida zin-3-one,
2-(2-cyanophenyl)-4-(3-pyridyl)-2,3,4,4a-tetrahydro-5H-(1)benzopyrano[4,3-c]pyridazin-3-one,
2-(2-bromophenyl)-4-(3-pyridyl)-2,3,4,4a-tetrahydro-5H-( I )benzopyrano[4,3-c]pyridazin-3-one, 2-(2-bromophenyl)-4-(2-hydroxyphenyl)-6-(2-pyridyl)-3(2H)-pyridazinone,
2-(²-bromophenyl)-4-(4-methoxyphenyl)-6-(2-pyridyl)-3(2H)-pyridazinone,
2-(2-cyanophenyl)-9-fluoro-5-hydroxy-4-phenyl-2,3-dihydro-5H-( I )benzopyrano[4,3-c]py ridazin-3-one,
2-(²-cyanophenyl)-9-fluoro-4-phenyl-2,3-dihydro-5H-(1)benzopyrano[4,3-c]pyridazin-3-o ne, 2-phenyl-6-(2-pyridyl)-4-(2-trifluoromethylsulfonyloxyphenyl)-3(2H)-pyridazinone,
2-(2-bromophenyl)-4-phenyl-6-(2-pyridyl)-3(2H)-pyridazinone,
2-(2-bromophenyl)-4-(3-pyridyl)-2,3-dihydro-5H-(1)benzopyrano[4,3-c]pyridazine-3-one,
2-(2-iodophenyl)-4-(3-pyridyl)-2,3-dihydro-5H-(1)benzopyrano[4,3-c]pyridazin-3-one,
2-phenyl-4-(3-pyridyl)-6-(2-pyridyl)-3 (²H)-pyridazinone,
4-(2-bromophenyl)-2-phenyl-6-(2-pyridyl)-3(2H)-pyridazinone,
2-(2-bromophenyl)-4-(3-pyridyl)-6-(2-pyridyl)-3(2H)-pyridazinone,
2-(2-chlorophenyl)-4-(4-morpholinoethylaminocarbonyl)-6-phenyl-3(2H)-pyridazinone,
2-(2-nitrophenyl)-4-(3-pyridyl)-6-(2-pyridyl)-3(2H)-pyridazinone,
2-(3-tolyl)-4-(3-pyridyl)-6-(2-pyridyl)-3 (2H)-pyridazinone,
2-(4-methanesulfonylphenyl)-4-(3-pyridyl)-6-(2-pyridyl)-3(2H)-pyridazinone,
2-(4-biphenyl)-4-(3-pyridyl)-6-(2-pyridyl)-3 (2H)-pyridazinone,
2-(2-naphthyl)-4-(3-pyridyl)-6-(2-pyridyl)-3(2H)-pyridazinone,
2-(3,4-methylenedioxyphenyl)-4-(3-pyridyl)-6-(2-pyridyl)-3(2H)-pyridazinone,
2-(3,4-dichlorophenyl)-4-(3-pyridyl)-6-(2-pyridyl)-3(2H)-pyridazinone,
2-(2-cyanophenyl)-4-phenyl-6-(2-pyrimidyl)-3(2H)-pyridazinone,
2-(2-pyridyl)-4-(2-pyridyl)-6-(2-methoxyphenyl)-3 (2H)-pyridazinone,
2-(3-formylphenyl)-4-(3-pyridyl)-6-(2-pyridyl)-3(2H)-pyridazinone,
2-(thiophen-3-yl)-4-(3-pyridyl)-6-(2-pyridyl)-3(2H)-pyridazinone,
2-(3-pyridyl)-4-phenyl-6-(2-pyridyl)-3 (2H)-pyridazinone,
2-(3-pyridyl)-4-phenyl-6-(2-pyrimidyl)-3(2H)-pyridazinone,
2-(2-methoxyphenyl)-4-(3-pyridyl)-6-(2-pyridyl)-4,5-dihydro-3(2H)-pyridazinone,
4-methyl-2,4,6-triphenyl-4,5-dihydro-3(2H)-pyridazinone,
2-(2-bromophenyl)-4-methyl-4,6-diphenyl-4,5-dihydro-3(2H)-pyridazinone,
2-(3-pyridin-1-oxide)-4-phenyl-6-(2-pyridyl)-3(2H)-pyridazinone,
2-(2-cyanopyridin-5-yl)-4-phenyl-6-(2-pyridyl)-3(2H)-pyridazinone,
2-(2-cyanopyridin-3-yl)-4-phenyl-6-(2-pyridyl)-3(2H)-pyridazinone,
2-(2-cyanopyridin-5-yl)-4-phenyl-6-(2-pyrimidyl)-3(2H)-pyridazinone,
2-(2-cyanopyridin-3-yl)-4-phenyl-6-(2-pyrimidyl)-3(2H)-pyridazinone,
2-(2-cyanophenyl)-4-phenyl-6-(2-pyrazyl)-3 (2H)-pyridazinone,
2-(2-cyanophenyl)-4-phenyl-6-(thiazol-2-yl)-3(2H)-pyridazinone,
2-(2-cyanophenyl)-4-methyl-4,6-diphenyl-4,5-dihydro-3(2H)-pyridazinone,
2-(2-bromophenyl)-4-(2-methoxyphenyl)-6-(2-pyridyl)-4,5-dihydro-1,2,4-triazin-3(2H)-on e,
2-(²-bromophenyl)-4-(2-hydroxyphenyl)-6-(2-pyridyl)-4,5-dihydro-1,2,4-triazin-3 (2H)-on e,
2-(²-cyanophenyl)-4-(2-methoxyphenyl)-6-(2-pyridyl)-4,5-dihydro-1,2,4-triazin-3(2H)-on e, 2-(2-bromophenyl)-4-phenyl-6-(2-pyridyl)-4,5-dihydro-1,2,4-triazin-3(2H)-one,
2-(2-bromophenyl)-6-(2-methoxyphenyl)-4-phenyl-4,5-dihydro-1,2,4-triazin-3(2H)-one,
2-(2-bromophenyl)-6-(2-hydroxyphenyl)-4-phenyl-4,5-dihydro-1,2,4-triazin-3(2H)-one,
2-(2-bromophenyl)-6-(2-dimethylaminoethoxyphenyl)-4-phenyl-4,5-dihydro-1,2,4-triazin-3(2H)-one,
2-(2-bromophenyl)-6-(2-methoxyphenyl)-4-(2-pyridyl)-4,5-dihydro-1,2,4-triazin-3(2H)-on e, 2-(2-cyanophenyl)-6-(2-hydroxyphenyl)-4-phenyl-4,5-dihydro-1,2,4-triazin-3(2H)-one,
2-(2-bromophenyl)-4-(2,5-dihydroxyphenyl)-6-(2-hydroxyphenyl)-4,5-dihydro-1,2,4-triazi n-3(2H)-one,
4-(2,5-dihydroxyphenyl)-6-(2-hydroxyphenyl)-2-phenyl-4,5-dihydro-1,2,4-triazin-3(2H)-o ne,
2-(2-cyanophenyl)-4-(2-hydroxyphenyl)-6-(2-pyridyl)-4,5-dihydro-1,2,4-triazin-3(2H)-on e, 2-(2-cyanophenyl)-6-(2-methoxyphenyl)-4-phenyl-4,5-dihydro-1,2,4-triazin-3(2H)-one,
4-(2-cyanophenyl)-6-(2-methoxyphenyl)-2-phenyl-4,5-dihydro-1,2,4-triazin-3(2H)-one,
2-(2-cyanophenyl)-6-(2-methoxyphenyl)-4-(2-pyridyl)-4,5-dihydro-1,2,4-triazin-3(2H)-on e, 2-(2-cyanophenyl)-4-phenyl-6-(2-pyridyl)-4,5-dihydro-1,2,4-triazin-3(2H)-one,
2-(2-cyanophenyl)-6-(2-pyridyl)-4-(thiophen-3-y)-4,5-dihydro-1,2,4-triazin-3(2H)-one,
2-(2-cyanophenyl)-6-(2-pyridyl)-4-(2-pyridyl)-4,5-dihydro-1,2,4-triazin-3(2H)-one,
2-(2-cyanophenyl)-6-(2-pyridyl)-4-(3-pyridyl)-4,5-dihydro-1,2,4-triazin-3(2H)-one,
4-(2-cyanophenyl)-2-phenyl-6-(2-pyridyl)-4,5-dihydro-1,2,4-triazin-3(2H)-one,
2-phenyl-6-(2-pyridyl)-4-(thiophen-3-yl)-4,5-dihydro-1,2,4-triazin-3(2H)-one,
2-(2-bromophenyl)-6-(2-pyridyl)-4-(thiophen-3-yl)-4,5-dihydro-1,2,4-triazin-3(2H)-one,
4-(2,4-dimethoxyphenyl)-2-phenyl-6-(2-pyridyl)-4,5-dihydro-1,2,4-triazin-3(2H)-one,
2-(²-bromophenyl)-6-(2-methoxyphenyl)-4-(thiophen-3-yl)-4,5-dihydro-1,2,4-triazin-3(2H)-one, 2-phenyl-6-(2-pyridyl)-4-(3-pyridyl)-4,5-dihydro-1,2,4-triazin-3(2H)-one,
2-(2-bromophenyl)-6-(2-pyridyl)-4-(3-pyridyl)-4,5-dihydro-1,2,4-triazin-3(2H)-one,
2-(2-bromophenyl)-4-(2-cyanophenyl)-6-(2-pyridyl)-4,5-dihydro-1,2,4-triazin-3(2H)-one,
2-(2-bromophenyl)-4,6-diphenyl-4,5-dihydro-1,2,4-triazin-3(2H)-one,
4-(2-bromophenyl)-2,6-diphenyl-4,5-dihydro-1,2,4-triazin-3(2H)-one,
2-(2-bromophenyl)-4-(2-bromophenyl)-6-phenyl-4,5-dihydro-1,2,4-triazin-3(2H)-one,
4-(2-bromophenyl)-6-(2-methoxyphenyl)-2-phenyl-4,5-dihydro-1,2,4-triazin-3(2H)-one,
2-(2-bromophenyl)-4-(2,5-dimethoxyphenyl)-6-(2-methoxyphenyl)-4,5-dihydro-1,2,4-tria zin-3(2H)-one,
4-(2,5-dimethoxyphenyl)-6-(2-methoxyphenyl)-2-phenyl-4,5-dihydro-1,2,4-triazin-3(2H)-one, 2-(2-bromophenyl)-6-(2-pyridyl)-4-(2-pyridyl)-4,5-dihydro-1,2,4-triazin-3(2H)-one,
2-phenyl-4-phenyl-6-(2-pyrimidyl)-4,5-dihydro-1,2,4-triazin-3(2H)-one,
2-(2-bromophenyl)-4-(4-biphenyl)-6-(2-pyridyl)-4,5-dihydro-1,2,4-triazin-3(2H)-one,
2-(2-bromophenyl)-4-(3-nitrophenyl)-6-(2-pyridyl)-4,5-dihydro-1,2,4-triazin-3(2H)-one,
2-(2-bromophenyl)-4-(4-fluorophenyl)-6-(2-pyridyl)-4,5-dihydro-1,2,4-triazin-3(2H)-one,
2-(2-bromophenyl)-4-(3-formylphenyl)-6-(2-pyridyl)-4,5-dihydro-1,2,4-triazin-3(2H)-one,
2-(2-bromophenyl)-4-(3-tolyl)-6-(2-pyridyl)-4,5-dihydro-1,2,4-triazin-3(2H)-one,
2-(2-bromophenyl)-4-(4-thiomethoxyphenyl)-6-(2-pyridyl)-4,5-dihydro-1,2,4-triazin-3(2H)-one,
2-(2-bromophenyl)-4-(2-chloropyridin-5-yl)-6-(2-pyridyl)-4,5-dihydro-1,2,4-triazin-3(2H)-one,
2-(2-cyanophenyl)-4-(3-nitrophenyl)-6-(2-pyridyl)-4,5-dihydro-1,2,4-triazin-3(2H)-one,
2-(2-cyanophenyl)-4-(3-aminophenyl)-6-(2-pyridyl)-4,5-dihydro-1,2,4-triazin-3(2H)-one,
and 2-(2-chlorophenyl)-4-phenyl-6-(2-pyrimidyl)-4,5-dihydro-1,2,4-triazin-3(2H)-one.

### Test Example 1

Suppressing Action to Calcium Influx into Nerve Cells Induced by AMPA

The suppressing action of the compounds of the present invention to calcium influx into nerve cells induced by AMPA was investigated using the primary culture system of nerve cells of cerebral cortex of embryo of rat.

Culturing Conditions:

Cerebral cortex was cut out from the brain of rat of gestational 18 days and treated with trypsin and DNase to disperse the cells. The cells were flown by MEM containing 10% of serum, sown in a culture bottle and astrocytes were proliferated. The astrocytes were re-dispersed by trypsin and sown in a 96-well plate. After incubation for one week, it was confirmed that the astrocytes covered all over the bottom and then the nerve cells of cerebral cortex which was dispersed by the above method were sown thereupon. After incubation for 24 hours, the medium was changed, the incubation was carried out for one week and, after that, the medium was changed to that containing 1 μM of MK-801. Nerve cells which were incubated for not shorter than 8 to 10 days were used.

Test Method:

Calcium influx into the cells was measured using Fura2-AM which was a calcium-sensitive fluorescent dye. It was treated in a medium containing Fura2-AM for 1 hour, incorporated into the cells, exchanged to a Tyrode solution containing 1 μMK-801 and stimulation was carried out using 2 μM AMPA. Change in the amount of calcium flown into the cells were measured as the change in the fluorescent intensity at the exciting wave length of 340/380 nm. Effect of the test compound was evaluated using the reaction resulted in the AMPA added to a Tyrode solution containing no compound as a control. As the control compound, GYK152466 (Le Peillet, et al., Brain Res., 571, 115, 1992) was used.

Results:

The compound (I) according to the present invention significantly inhibited the calcium influx into nerve cells induced by AMPA (Table 1). The IC₅₀ of GYKI52466 was 9.02 μM.

TABLE 1

| Ex. No. | IC₅₀ (μM) |
|---|---|
| 1 | 0.1 |
| 2 | 0.1 |
| 3 | 0.2 |
| 4 | 0.06 |
| 5 | 6.7 |
| 6 | 0.1 |
| 7 | 0.2 |
| 8 | 0.1 |
| 9 | 0.02 |
| 11 | 9.9 |
| 12 | 3.9 |
| 13 | 0.3 |
| 14 | 0.2 |
| 15 | 0.2 |
| 16 | 0.7 |
| 17 | 0.2 |
| 18 | 0.1 |
| 19 | 0.06 |
| 20 | 0.1 |
| 21 | 0.5 |
| 22 | 2.7 |
| 23 | 0.1 |
| 24 | 0.06 |
| 25 | 0.2 |
| 26 | 0.3 |
| 27 | 0.04 |
| 28 | 0.1 |
| 29 | 0.1 |
| 30 | 0.2 |
| 31 | 0.9 |
| 32 | 0.1 |
| 33 | 0.07 |
| 34 | 0.3 |
| 35 | 3.0 |
| 36 | 4.6 |
| 37 | 0.1 |
| 38 | 0.4 |
| 39 | 0.05 |
| 40 | 0.1 |
| 41 | 0.03 |
| 42 | 0.1 |
| 43 | 0.1 |
| 44 | 0.2 |
| 45 | 0.2 |
| 46 | 0.3 |
| 47 | 0.2 |
| 48 | 0.1 |
| 49 | 0.07 |
| 50 | 0.1 |
| 51 | 0.8 |
| 52 | 0.2 |
| 53 | 0.5 |
| 54 | 0.1 |
| 55 | 0.8 |
| 56 | 0.2 |
| 57 | 0.2 |
| 58 | 0.4 |
| 59 | 0.6 |
| 60 | 0.2 |
| 61 | 0.3 |
| 62 | 4.0 |
| 63 | 0.3 |
| 64 | 0.7 |
| 70 | 0.8 |
| 73 | 1.1 |
| 74 | 0.9 |
| 75 | 0.7 |
| 76 | 6.2 |
| 79 | 7.2 |
| 80A | 7.1 |
| 80B | 0.2 |
| 81B | 0.7 |
| 82 | 1.2 |
| 83 | 0.6 |
| 85 | 0.2 |
| 86 | 0.1 |
| 87 | 0.05 |
| 88 | 0.1 |
| 89 | 5.0 |
| 90 | 0.9 |
| 91 | 0.3 |
| 92 | 0.1 |
| 93 | 0.03 |
| 94 | 0.9 |
| 95 | 0.05 |
| 96 | 0.6 |
| 97 | 0.7 |
| 98 | 0.4 |
| 99 | 0.07 |
| 100 | 0.05 |
| 101 | 0.1 |
| 102 | 0.1 |
| 103 | 0.1 |
| 104 | 0.5 |
| 105 | 0.2 |
| 106 | 0.1 |
| 107 | 0.3 |
| 108 | 4.0 |
| 109 | 0.3 |
| 110 | 0.1 |
| 111 | 0.4 |
| 112 | 0.3 |
| 113 | 7.1 |
| 115 | 7.2 |
| 118 | 0.03 |
| 119 | 4.3 |
| 121 | 0.4 |
| 122 | 0.2 |
| 123 | 0.3 |
| 124 | 0.2 |
| 125 | 0.6 |
| 127 | 0.4 |
| 128 | 0.1 |
| 129 | 3.0 |

### Test Example 2

Anticonvulsant Action Induced by AMPA

A test compound was suspended in a 0.5% methyl cellulose solution or in sesame oil and was orally administered (25 mg/kg) to male mice of ddy strain. After 30 minutes or 1 hour from the oral administration, AMPA was continuously injected (2 nmole/5μl/minute/mouse) into lateral ventricle to induce the convulsions. The effect was judged by a time-extending action until the convulsion takes place by a continuous injection of AMPA.

Results:

The compound (I) according to the present invention showed an excellent anticonvulsant action. For example, the compounds of Examples 9, 29, 45, 59, 88, 97, 100, 102 and 103 significantly inhibited the convulsion induced by AMPA.

### Test Example 3

Middle Cerebral Artery Occlusion Model

The usefulness of the compound related to the present invention in the remedy of cerebral vascular accident acute stage was confirmed by the test below. Namely, the cerebral bloodstream of middle cerebral artery was blocked by inserting a nylon suture thread of 4-0 specification whose edge was crashed with flame, by 17 mm from the branch of internal carotid artery, through internal carotid artery from the external carotid artery of a male Sprange Dawley rat, and cerebral infarction was prepared (Zea Longa et al., Stroke 20:84-91, 1989). The size of the cerebral infarction was evaluated by preparing the intersection slice of brain having a thickness of 2 mm and measuring the area of a portion which was not stained by TTC staining. The effect of the tested substance was carried out in this model by comparing the infarction size between a group treated with a solvent and a group treated with the tested substance.

As a result, the compound (I) according to the present invention revealed an excellent effect as the therapeutic agent of cerebral vascular accident acute stage.

### Test Example 4

Antimethamphetamine Effect

(S)-(+)-N,α-dimethylphenetylamine (hereinafter, referred to as “methamphetamine”) was dosed intraperitoneal injection to a rat or mouse to which the tested compound was dosed, and a quantity of active movement was measured using an active movement measuring apparatus (SCANET SV-10; manufactured by TOYO Sangyo Co., Ltd.). The activity as the therapeutic agent of schizophrenia was evaluated using the hyperdynamic effect control of movement caused by methamphetamine as an index (K. E. Vanover, Psychopharmacology 136: 123-131, 1998). The effect of the tested substance was confirmed by the control effect of a quantity of movement accentuation in comparison with the group dosed with a solvent.

As a result, the compound (I) according to the present invention revealed an excellent anti-methamphetamine effect.

### Test Example 5

Intercolliculus Decerebrated Rigidity Model

An animal model in which the myotony of limbs was provoked was prepared by electrically sectioning between the colliculus superior and the colliculus inferior of a rat. Myorelaxation effect was evaluated based on the effect of controlling the increase of muscle discharge which is generated when the posterior limbs in this model are moved back and forth. The effect of the tested substance was confirmed by the changes of muscle discharge amount before dosing the tested substance and muscle discharge amount after dosing it.

The compound (I) according to the present invention revealed an excellent myorelaxation effect.

### Test Example 6

Light Dark Test

A mouse is put in a dark box which is composed of two light and dark boxes which are linked by a tunnel, and items below were recorded concerning the behavior of the mouse for 5 minutes after that.

1. A time for remaining in the light and dark boxes.

2. Times by which the mouse went and came back between the light box and the dark box.

3. Times by which the mouse went until the entrance of the light box.

The antianxiety effect of the tested compound was detected as the elongation of the time remaining in the light box, the increase of times by which the mouse went and came back between the light box and the dark box, and the increase of times by which the mouse went until the entrance of the light box, for the group dosed with a solvent (Hascoet M., Bourin M., Pharm. Biochem. Behav. 60: 645-653, 1998).

The compound (I) according to the present invention showed an excellent antianxiety effect.

### Test Example 7

6-OHDA-Induced Hemi-Parkinsonian Rat Model of Parkinson's Disease

10 mg/kg of L-Dihydroxyphenylalanine (L-DOPA) (twice per day) was intraperitaneously dosed every day to the rat whose one side of nigra was destroyed by injecting 6-hydroxydopamine (6-OHDA) into medial forebrain bundle. The increase of contralateral rotation to the intact side was provoked (C. Marin et al, Synapse 36(4):267-274, 2000). After the solvent or the tested compound was dosed to the rat, influence on the provoked rotation was studied.

The compound (I) of the present invention as the test sample delayed the time until the maximum rotatory response after dosing L-DOPA, and increased the time of showing rotation which is a half or more of the maximum rotational number.

### Test Example 8

Acetic Acid Writhing Model

Anguishing condition under which the lower half of mice's body was twisted, its abdomen was dented and its hind legs were extended was provoked by injecting 0.6% acetic acid in saline into the abdomen of the mice. After the tested compound and the solvent were dosed, the acetic acid in saline was injected into the abdomen, and analgesic effect was evaluated by comparing the times of these abnormal actions within an observation time (5 to 15 minutes after the dose of acetic acid) which occur after the dosing (Basic Pharmacology Experiment, edited by Kazuhiko Kubota, pages 45-47, Nankoh-do).

As a result, it could be confirmed that the compound (I) according to the present invention controls the times of the abnormal actions significantly and has an excellent analgesic effect.

### Test Example 9

Vomiting Model Induced by Cisplatin

A intravenous catheter was buried in a ferret, and the rat was postoperatively recovered. Then, vomiting reaction was provoked by injecting 10 mg/kg of cis-diaminedichloroplatinum (cisplatin) (A. Fink-Jensen et al., Neuroscience Letters 137: 173-177, 1992). Cisplatin (10 mg/kg) was injected a ferret which was treated with the tested compound or the solvent, then the ferret was put in an observation cage, and times of the rhythmical contraction of abdomen (defined as vomiting) and times until vomiting occurs during the observation period of 240 minutes were measured.

As a result, the compound (I) according to the present invention reduced the latent time and the vomiting times significantly.

### Test Example 10

Experimental Autoimmune Encephalomyelitis Model

Female Lewis rats (205±10 g) obtained from Charles River, Kent UK, were housed in pairs under environmentally controlled conditions (6:00a.m.-6:00p.m. light/dark cycle; 22-24° C.; 45-55% humidity) and allowed free access to food and water. Experimental groups consisted of 9-12 animals. Rats were immunized with 20-50 μl of inoculum containing 50 μg guinea pig myelin basic protein (MBP; final concentration 2 mg/ml), emulsified in Freund's complete adjuvant (CFA; Sigma, UK) containing Mycobacterium tuberculosis H37Ra (final concentration 5.5 mg/ml; Difco Laboratories, UK). Animals were weighed and monitored daily and clinical disease scored as (0) no clinical signs; (1) flaccid tail and weight loss; (2) hind limb hypotonia with further weight loss; (3) complete hind limb paralysis; (4) paraplegia and (5) death. In addition, intermediate scores were assigned to animals which showed a loss of tonicity in the distal half of the tail (score=0.5), paralysis of one hind limb (score=2.5) or complete hind limb paralysis with forelimb weakness (score=3.5). During the period of compound administration (10-16 days post immunisation; dpi) animals were scored 15h after injection of vehicle or compound to avoid any acute effect of treatment on disease score. Compounds were dissolved/suspended in 0.5% methyl cellulose using a hand held Polytron homogeniser (PT1200; 2 min). Rats were dosed p.o. with either methyl cellulose vehicle (2.5 ml/kg) or compound at 5, 10 and 20 mg/kg.

Results:

The compound of the invention is improved in view of experimental autoimmune encephalomyelitis. The compound (I) according to the present invention showed a superior effect to the vehicle-administered group.

1. A compound represented by the following formula (I), a salt thereof or a hydrate thereof: 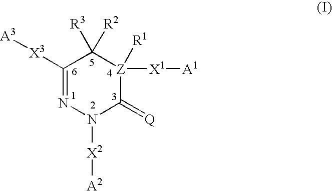
wherein in formula (I) A¹, A² and A³ are independent of each other, and wherein
each of A² and A³ represents a C₃₋₈ cycloalkyl group, a C₃₋₈ cycloalkenyl group, a 5- to 14-membered non-aromatic heterocyclic group, a C₆₋₁₄ aromatic hydrocarbon cyclic group or a 5- to 14-membered aromatic heterocyclic group, each of which is optionally substituted, and
A¹ represents a C₃₋₈ cycloalkyl group, a C₃₋₈ cycloalkenyl group, a C₆₋₁₄ aromatic hydrocarbon cyclic group or a 5- to 14-membered aromatic heterocyclic group, each of which is optionally substituted,
wherein at least one of A² and A³ always represents the 5- to 14-membered non-aromatic heterocyclic group (provided that piperidinyl group, 2,4-dihydropyrazol-3-one-yl group and pyrimidinetrione-yl group are excluded) or at least one of A¹, A² and A³ represents the 5- to 14-membered aromatic heterocyclic group (provided that indolyl group and pyrazolyl group are excluded);
Q represents O, S or NH;
Z represents C;
X¹, X² and X³ are independent of each other, and wherein
X¹ represents a single bond, an optionally substituted C₁₋₆ alkylene group, an optionally substituted C₂₋₆ alkenylene group, an optionally substituted C₂₋₆ alkynylene group, —O—, —N(R⁴)CO—, —CON(R⁵)—, —CH₂N(R⁷)—, —CH₂CO—, —COCH₂—, —N(R⁸)SO₀₋₂—, —SO₀₋₂N(R⁹)—, —CH₂SO₀₋₂—, —SO₀₋₂CH₂—, —CH₂—, —OCH₂—, —N(R¹⁰)CON(R¹¹)—, —N(R¹²)CS—N(R¹³)— or —SO₀₋₂—, wherein R⁴, R⁵, R⁷, R⁸, R⁹, R¹⁰, R¹¹, R¹² and R¹³ are independent of each other and each represents a hydrogen atom, a C₁₋₆ alkyl group or a C₁₋₆ alkoxy group,
X² represents a single bond, —NH—, —O—, —CON(R⁵)—, —N(R⁶)CH₂—, —CH₂N(R⁷)—, —N(R⁸)SO₀₋₂—, —SO₀₋₂N(R⁹)—, —CH₂SO₀₋₂—, —SO₀₋₂CH₂—, —CH₂O—, —OCH₂—, —N(R¹⁰)CON(R¹¹)—, —N(R¹²)CS—N(R¹³)— or —SO₀₋₂—, wherein R⁵, R⁶, R⁷, R⁸, R⁹, R¹⁰, R¹¹, R¹² and R¹³ are independent of each other and each represents a hydrogen atom, a C₁₋₆ alkyl group or a C₁₋₆ alkoxy group, and
X³ represents a single bond, an optionally substituted C₁₋₆ alkylene group, an optionally substituted C₂₋₆ alkenylene group, an optionally substituted C₂₋₆ alkynylene group, —NH—, —O—, —N(R⁴)CO—, —CON(R⁵)—, —N(R⁶)CH₂—, —CH₂N(R⁷)—, —CH₂CO—, —COCH₂—, —N(R⁸)SO₀₋₂—, —SO₀₋₂N(R⁹)—, —CH₂SO₀₋₂—, —SO₀₋₂CH₂—, —CH₂O—, —OCH₂—, —N(R¹⁰)CON(R¹¹)—, —N(R¹²)CS—N(R¹³)— or —SO₀₋₂—, wherein R⁴, R⁵, R⁶, R⁷, R⁸, R⁹, R¹⁰, R¹¹, R¹² and R¹³ are independent of each other and each represents a hydrogen atom, a C₁₋₆ alkyl group or a C₁₋₆ alkoxy group;
R¹ and R² are independent of each other and each represents a hydrogen atom, an optionally substituted C₁₋₆ alkyl group, an optionally substituted C₂₋₆ alkenyl group or an optionally substituted C₂₋₆ alkynyl group, or R¹ and R² may be bound to each other such that CR²-ZR¹ forms a carbon-carbon double bond represented by C═C;
R³ represents a hydrogen atom, an optionally substituted C₁₋₆ alkyl group, an optionally substituted C₂₋₆ alkenyl group or an optionally substituted C₂₋₆ alkynyl group.

2. The compound according to claim 1, a salt thereof or a hydrate thereof, wherein A¹, A² and/or A³ are independent of each other and each represents a C₃₋₈ cycloalkyl group, or a C₃₋₈ cycloalkenyl group, or A² and/or A³ represents a 5- to 14-membered non-aromatic heterocyclic group, each of which is optionally substituted.

3. The compound according to claim 1, a salt thereof or a hydrate thereof, wherein A¹, A² and A³ are independent of each other and each represents a C₆₋₁₄ aromatic hydrocarbon cyclic group or 5- to 14-membered aromatic heterocyclic group, each of which is optionally substituted.

4. The compound according to claim 1, a salt thereof or a hydrate thereof, wherein A¹, A² and A³ are independent of each other and each represents a phenyl group, pyrrolyl group, pyridyl group, pyridazinyl group, pyrimidinyl group, pyrazinyl group, thienyl group, thiazolyl group, furyl group, naphthyl group, quinolyl group, isoquinolyl group, benzimidazolyl group, benzothiazolyl group, benzoxazolyl group, imidazopyridyl group, carbazolyl group, cyclopentyl group, cyclohexyl group, cyclohexenyl group, dioxinyl group, adamantly group, pyrrolidinyl group, piperazinyl group or morpholinyl group, each of which is optionally substituted.

5. The compound according to claim 1, a salt thereof or a hydrate thereof, wherein A¹, A² and A³ are independent of each other and each represents a group represented by the formula: 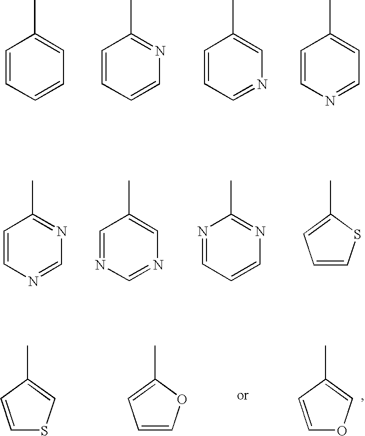
each of which is optionally substituted.

6. The compound according to claim 1, a salt thereof or a hydrate thereof,
wherein the substituent that is optionally substituted on the C₁₋₆ alkylene group, C₂₋₆ alkenylene group or C_{2≢}alkynylene group of X¹ or X³ is selected from the group consisting of a hydroxyl group, a halogen atom and a cyano group; and
the substituent that is optionally substituted on the C₃₋₈ cycloalkyl group, C₃₋₈ cycloalkenyl group, 5- to 14-membered non-aromatic heterocyclic group, C₆₋₁₄ aromatic hydrocarbon cyclic group or 5- to 14-membered aromatic heterocyclic group of A¹, A² or A³ is selected from the group consisting of (1) a hydroxyl group, (2) a halogen atom, (3) a nitrile group, (4) a nitro group, (5) a C₁₋₆ alkyl group, C₂₋₆ alkenyl group or C₂₋₆ alkynyl group, each of which is optionally substituted with at least one group selected from the group consisting of a hydroxyl group, nitrile group, halogen atom, C₁₋₆ alkylamino group, di(C₁₋₆ alkyl)amino group, C₂₋₆ alkenylamino group, di(C₂₋₆ alkenyl)amino group, C₂₋₆ alkynylamino group, di(C₂₋₆ alkynyl)amino group, N—C₁₋₆ alkyl-N-C₂₋₆ alkenylamino group, N—C₁₋₆ alkyl-N—C₂₋₆ alkynylamino group, N—C₂₋₆ alkenyl-N—C₂₋₆ alkynylamino group, aralkyloxy group, TBDMS oxy group, C₁₋₆ alkylsulfonylamino group, C₁₋₆ alkylcarbonyloxy group, C₂₋₆ alkenylcarbonyloxy group, C₂₋₆ alkynylcarbonyloxy group, N—C₁₋₆ alkylcarbamoyl group, N—C₂₋₆ alkenylcarbamoyl group and N—C₁₋₆ alkynylcarbamoyl group, (6) a C₁₋₆ alkoxy group, C₂₋₆ alkenyloxy group or C₂₋₆ alkynyloxy group, each of which is optionally substituted with at least one group selected from the group consisting of a C₁₋₆ alkylamino group, aralkyloxy group and hydroxyl group, (7) a C₁₋₆ alkylthio group, C₂₋₆ alkenylthio group or C₂₋₆ alkynylthio group, each of which is optionally substituted with at least one group selected from the group consisting of a hydroxyl group, nitrile group, halogen atom, C₁₋₆ alkylamino group, aralkyloxy group, TBDMS oxy group, C₁₋₆ alkylsulfonylamino group, C₁₋₆ alkylcarbonyloxy group and C₁₋₆ alkylcarbamoyl group, (8) a carbonyl group substituted with a group selected from the group consisting of a C₁₋₆ alkoxy group, amino group, C₁₋₆ alkylamino group, di(C₁₋₆ alkyl)amino group, C₂₋₆ alkenylamino group, di(C₂₋₆ alkenyl)amino group, C₂₋₆ alkynylamino group, di(C₂₋₆ alkynyl)amino group, N—C₁₋₆ alkyl-N—C₂₋₆ alkenylamino group, N—C₁₋₆ alkyl-N—C₂₋₆ alkynylamino group and N—C₂₋₆ alkenyl-N—C₂₋₆ alkynylamino group, (9) an amino group which is optionally substituted with one or two groups selected from the group consisting of a C₁₋₆ alkyl group, C₂₋₆ alkenyl group, C₂₋₆ alkynyl group, C₁₋₆ alkylsulfonyl group, C₂₋₆ alkenylsulfonyl group, C₂₋₆ alkynylsulfonyl group, C₁₋₆ alkylcarbonyl group, C₂₋₆ alkenylcarbonyl group and C₂₋₆ alkynylcarbonyl group, (10) a C₁₋₆ alkylsulfonyl group, (11) a C₂₋₆ alkenylsulfonyl group, (12) a C₂₋₆ alkynylsulfonyl group, (13) a C₁₋₆ alkylsulfinyl group, (14) a C₂₋₆ alkenylsulfinyl group, (15) a C₂₋₆ alkynylsulfinyl group, (16) a formyl group, (17) a C₃₋₈ cycloalkyl group or C₃₋₈ cycloalkenyl group, each of which is optionally substituted with at least one group selected from the group consisting of a hydroxyl group, halogen atom, nitrile group, C₁₋₆ alkyl group, C₁₋₆ alkoxy group, C₁₋₆ alkoxy-C₁₋₆ alkyl group and aralkyl group, (18) a 5- to 14-membered non-aromatic heterocyclic group which is optionally substituted with at least one group selected from the group consisting of a hydroxyl group, halogen atom, nitrile group, C₁₋₆ alkyl group, C₁₋₆ alkoxy group, C₁₋₆ alkoxy-C₁₋₆ alkyl group and aralkyl group, (19) a C₆₋₁₄ aromatic hydrocarbon cyclic group which may be substituted with at least one group selected from the group consisting of a hydroxyl group, halogen atom, nitrile group, C₁₋₆ alkyl group, C₁₋₆ alkoxy group, C₁₋₆ alkoxy-C₁₋₆ alkyl group and aralkyl group, and (20) a 5- to 14-membered aromatic heterocyclic group which is optionally substituted with at least one group selected from the group consisting of a hydroxyl group, halogen atom, nitrile group, C₁₋₆ alkyl group, C₁₋₆ alkoxy group, C₁₋₆ alkoxy-C₁₋₆ alkyl group and aralkyl group, and (21) thiol group.

7. The compound according to claim 1, a salt thereof or a hydrate thereof, wherein substituent groups on A¹, A² and/or A³ are independent of each other and each represents a hydroxyl group, a halogen atom, a nitrile group or a nitro group.

8. The compound according to claim 1, a salt thereof or a hydrate thereof, wherein Q is O.

9. The compound according to claim 1, a salt thereof or a hydrate thereof, wherein X² is a single bond and X¹ and X³ are independent of each other and each represents a single bond, —CH₂—, —CH(OH)—, —CH₂CH₂—, —CH═CH—, or —C═C—.

10. The compound according to claim 1, a salt thereof or a hydrate thereof, wherein X¹, X² and X³ each represents a single bond.

11. The compound according to claim 1, a salt thereof or a hydrate thereof, wherein R¹, R² and/or R³ represent an optionally substituted C₁₋₆ alkyl group.

12. The compound according to claim 1, a salt thereof or a hydrate thereof, wherein R¹, R² and/or R³ each represents a hydrogen atom.

13. The compound according to claim 1, a salt thereof or a hydrate thereof, wherein R¹ and R² are bound to each other such that the partial structure ZR¹—CR² forms a carbon-carbon double bond represented by the formula C═C.

14. The compound according to claim 1, a salt thereof or a hydrate of them, wherein R³ is bound to an atom in A¹ to form a ring with the atom and X¹.

15. The compound according to claim 1, a salt thereof or a hydrate of them, wherein R³ is bound to an atom in A³ to form a ring with the atom and X³.

16. The compound according to claim 14 or 15, a salt thereof or a hydrate of them, wherein the ring formed by R³ is (1) an optionally substituted C₅₋₈ hydrocarbon ring or (2) a 5- to 8-membered heterocyclic ring which contains an oxygen atom and is optionally substituted.

17. The compound according to claim 14 or 15, a salt thereof or a hydrate of them, wherein X³ is a single bond.

18. The compound according to claim 1, a salt thereof or a hydrate of them, wherein the binding positions of substituent groups on A¹, A² and/or A³ are α-positions of the carbon atoms on A¹, A² and/or A³, each of which are bound to X¹, X² and X³, respectively.

19. The compound according to claim 1, a salt thereof or a hydrate thereof, which is represented by the formula (II): 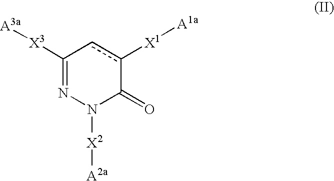
wherein A^{1a}, A^{2a} and A^{3a} are independent of each other and each represents a C₆₋₁₄ aromatic hydrocarbon cyclic group or a 5- to 14-membered aromatic heterocyclic group, each of which is optionally substituted; wherein at least one of A^{1a}, A^{2a} and A^{3a} always represents the 5- to 14-membered aromatic heterocyclic group (provided that indolyl group and pyrazolyl group are excluded);
X¹, X² and X³ have the same meanings as defined in the above-mentioned claim 1, respectively; and
the partial structure: [symbol]
represents a single or double bond.

20. The compound according to claim 19, a salt thereof or a hydrate thereof, wherein A¹, A² and A³a are independent of each other and each represents a phenyl group, pyrrolyl group, pyridyl group, pyridazinyl group, pyrimidinyl group, pyrazinyl group, thienyl group, thiazolyl group, furyl group, naphthyl group, quinolyl group, isoquinolyl group, benzimidazolyl group, benzothiazolyl group, benzoxazolyl group, imidazopyridyl group, carbazolyl group, cyclopentyl group, cyclohexyl group, cyclohexenyl group, dioxinyl group, adamantyl group, each of which is optionally substituted.

21. The compound according to claim 19, a salt thereof or a hydrate thereof, wherein X¹, X² and X³ each represents a single bond.

22. The compound according to claim 1, a salt thereof or a hydrate of them, which is represented by the formula: 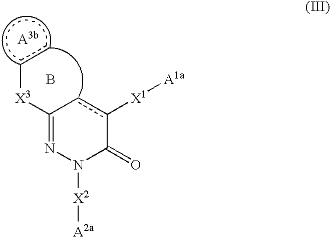
wherein the partial structure: [symbol]
represents a single bond or a double bond;
A^{1a} and A^{2a} are independent of each other and each represents a C₆₋₁₄ aromatic hydrocarbon cyclic group or a 5- to 14-membered aromatic heterocyclic group, each of which is optionally substituted; wherein at least one of A^{1a} and A^{2a} always represents the 5- to 14-membered aromatic heterocyclic group (provided that indolyl group and pyrazolyl group are excluded);
X¹, X² and X³ are independent of each other, and wherein
X¹ represents a single bond, an optionally substituted C₁₋₆ alkylene group, an optionally substituted C₂₋₆ alkenylene group, an optionally substituted C₂₋₆ alkynylene group, —O—, —N(R⁴)CO—, —CON(R⁵)—, —CH₂N(R⁷)—, —CH₂CO—, —COCH₂—, —N(R⁸)SO₀₋₂—, —SO₀₋₂N(R⁹)—, —CH₂SO₀₋₂—, —SO₀₋₂CH₂—, —CH₂—, —OCH₂—, —N(R¹⁰)CON(R¹¹)—, —N(R¹²)CS—N(R¹³)— or —SO₀₋₂—, wherein R⁴, R⁵, R⁷, R⁸, R⁹, R¹⁰, R¹¹, R¹² and R¹³ are independent of each other and each represents a hydrogen atom, a C₁₋₆ alkyl group or a C₁₋₆ alkoxy group,
X² represents a single bond, —NH—, —O—, —CON(R⁵)—, —N(R⁶)CH₂—, —CH₂N(R⁷)—, —N(R⁸)SO₀₋₂—, —SO₀₋₂N(R⁹)—, —CH₂SO₀₋₂—, —SO₀₋₂CH₂—, —CH₂O—, —OCH₂—, —N(R¹⁰)CON(R¹¹)—, —N(R¹²)CS—N(R¹³)— or —SO₀₋₂—, wherein R⁵, R⁶, R⁷, R⁸, R⁹, R¹⁰, R¹¹, R¹² and R¹³ are independent of each other and each represents a hydrogen atom, a C₁₋₆ alkyl group or a C₁₋₆ alkoxy group, and
X³ represents a single bond, an optionally substituted C₁₋₆ alkylene group, an optionally substituted C₂₋₆ alkenylene group, an optionally substituted C₂₋₆ alkynylene group, —NH—, —O—, —N(R⁴)CO—, —CON(R⁵)—, —N(R⁶)CH₂—, —CH₂N(R⁷)—, —CH₂CO—, —COCH₂—, —N(R⁸)SO₀₋₂—, —SO₀₋₂N(R⁹)—, —CH₂SO₀₋₂—, —SO₀₋₂CH₂—, —CH₂O—, —OCH₂—, —N(R¹⁰)CON(R¹¹)—, —N(R¹²)CS—N(R¹³)— or —SO₀₋₂—, wherein R⁴, R⁵, R⁶, R⁷, R⁸, R⁹, R¹⁰, R¹¹, R¹² and R¹³ are independent of each other and each represents a hydrogen atom, a C₁₋₆ alkyl group or a C₁₋₆ alkoxy group;
the ring A^{3b} represents a C₆₋₈ aromatic hydrocarbon ring or a 5- to 8-membered aromatic heterocyclic ring, each of which may be substituted; and the ring B represents (1) an optionally substituted C₅₋₉ cycloalkane or C₅₋₉ cycloalkene or (2) a 5- to 9-membered non-aromatic heterocyclic ring which contains a hetero atom selected from the group consisting of N, O and S, and is optionally substituted.

23. The compound according to claim 22, a salt thereof or a hydrate of them, wherein A^{1a}, A^{2a} and A^{3b} are independent of each other and each represents a phenyl group, pyrrolyl group, pyridyl group, pyridazinyl group, pyrimidinyl group, pyrazinyl group, thienyl group, thiazolyl group, furyl group, naphthyl group, quinolyl group, isoquinolyl group, indolyl group, benzimidazolyl group, benzothiazolyl group, benzoxazolyl group, imidazopyridyl group, carbazolyl group, cyclopentyl group, cyclohexyl group, cyclohexenyl group, dioxinyl group, adamantly group, pyrrolidinyl group, piperidinyl group, piperazinyl group or morpholinyl group, each of which is optionally substituted.

24. The compound according to claim 1, a salt thereof or a hydrate of them, which represented by the formula: 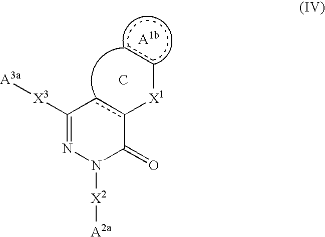
wherein the partial structure: [symbol]
represents a single bond or double bond;
A^{2a} and A^{3a} are independent of each other and each represents a C₆₋₁₄ aromatic hydrocarbon cyclic group or a 5- to 14-membered aromatic heterocyclic group, each of which is optionally substituted; wherein at least one of A^{2a} and A^{3a} always represents the 5- to 14-membered aromatic heterocyclic group (provided that indolyl group and pyrazolyl group are excluded);
X¹, X² and X³ are independent of each other, and wherein
X¹ represents a single bond, an optionally substituted C₁₋₆ alkylene group, an optionally substituted C₂₋₆ alkenylene group, an optionally substituted C₂₋₆ alkynylene group, —O—, —N(R⁴)CO—, —CON(R⁵)—, —CH₂N(R⁷)—, —CH₂CO—, —COCH₂—, —N(R⁸)SO₀₋₂—, —SO₀₋₂N(R⁹)—, —CH₂SO₀₋₂—, —SO₀₋₂CH₂—, —CH₂—, —OCH₂—, —N(R¹⁰)CON(R¹¹)—, —N(R¹²)CS—N(R¹³)— or —SO₀₋₂—, wherein R⁴, R⁵, R⁷, R⁸, R⁹, R¹⁰, R¹¹, R¹² and R¹³ are independent of each other and each represents a hydrogen atom, a C₁₋₆ alkyl group or a C₁₋₆ alkoxy group,
X² represents a single bond, —NH—, —O—, —CON(R⁵)—, —N(R⁶)CH₂—, —CH₂N(R⁷)—, —N(R⁸)SO₀₋₂—, —SO₀₋₂N(R⁹)—, —CH₂SO₀₋₂—, —SO₀₋₂CH₂—, —CH₂O—, —OCH₂—, —N(R¹⁰)CON(R¹¹)—, —N(R¹²)CS—N(R¹³)— or —SO₀₋₂—, wherein R⁵, R⁶, R⁷, R⁸, R⁹, R¹⁰, R¹¹, R¹² and R¹³ are independent of each other and each represents a hydrogen atom, a C₁₋₆ alkyl group or a C₁₋₆ alkoxy group, and
X³ represents a single bond, an optionally substituted C₁₋₆ alkylene group, an optionally substituted C₂₋₆ alkenylene group, an optionally substituted C₂₋₆ alkynylene group, —NH—, —O—, —N(R⁴)CO—, —CON(R⁵)—, —N(R⁶)CH₂—, —CH₂N(R⁷)—, —CH₂CO—, —COCH₂—, —N(R⁸)SO₀₋₂—, —SO₀₋₂N(R⁹)—, —CH₂SO₀₋₂—, —SO₀₋₂CH₂—, —CH₂O—, —OCH₂—, —N(R¹⁰)CON(R¹¹)—, —N(R¹²)CS—N(R¹³)— or —SO₀₋₂—, wherein R⁴, R⁵, R⁶, R⁷, R⁸, R⁹, R¹⁰, R¹¹, R¹² and R¹³ are independent of each other and each represents a hydrogen atom, a C₁₋₆ alkyl group or a C₁₋₆ alkoxy group;
the ring A^{1b} represents a C₆₋₈ aromatic hydrocarbon ring or a 5- to 8-membered aromatic heterocyclic ring, each of which may be substituted; and the ring C represents (1) an optionally substituted C₅₋₉ cycloalkane or C₅₋₉ cycloalkene or (2) a 5- to 9-membered non-aromatic heterocyclic ring which contains a hetero atom selected from the group consisting of N, O and S, and is optionally substituted.

25. The compound according to claim 24, a salt thereof or a hydrate of them, wherein A^{1b}, A^{2a} and A^{3a} are independent of each other and each represents a phenyl group, pyrrolyl group, pyridyl group, pyridazinyl group, pyrimidinyl group, pyrazinyl group, thienyl group, thiazolyl group, furyl group, naphthyl group, quinolyl group, isoquinolyl group, indolyl group, benzimidazolyl group, benzothiazolyl group, benzoxazolyl group, imidazopyridyl group, carbazolyl group, cyclopentyl group, cyclohexyl group, cyclohexenyl group, dioxinyl group, adamantly group, pyrrolidinyl group, piperidinyl group, piperazinyl group or morpholinyl group, each of which is optionally substituted.

26. The compound according to claim 22, a salt thereof or a hydrate of them, which is represented by the formula: 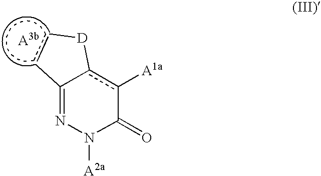
wherein A^{1a}, A^{2a}, A^{3b} and the partial structure: [symbol]
have the same meanings as defined in the above-mentioned claim 22; and D represents a group represented by —CH₂—, —(CH₂)₂—, —C═C—, —C≡C—, —O—, —OCH₂—, —CH₂O—, —SO₀₋₂, —SCH₂—, —CH₂S—, —SOCH₂—, —CH₂SO—, —SO₂CH₂—, —CH₂SO₂—, —NR¹⁴—, —NR¹⁴CH₂— or —CH₂NR¹⁴— (wherein, R¹⁴ represents a hydrogen atom, a C₁₋₆ alkyl group, an optionally substituted C₃₋₈ cycloalkyl group, an optionally substituted 5- to 14-membered non-aromatic heterocyclic group, an optionally substituted C₆₋₁₄ aromatic hydrocarbon cyclic group or an optionally substituted 5- to 14-membered aromatic heterocyclic group), and the substitutable positions in D is optionally substituted.

27. The compound according to claim 24, a salt thereof or a hydrate of them, which is represented by the formula: 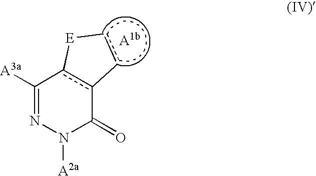
wherein A^{1b}, A^{2a}, A^{3a} and the partial structure: [symbol]
have the same meanings as defined in the above-mentioned claim 24, respectively; and E represents —CH₂—, —(CH₂)₂—, —C═C—, —C≡C—, —O—, —OCH₂—, —CH₂O—, —SO₀₋₂—, —SCH₂—, —CH₂S—, —SOCH₂—, —CH₂SO—, —SO₂CH₂—, —CH₂SO₂—, —NR¹⁴—, —NR¹⁴CH₂— or —CH₂NR¹⁴— (wherein, R¹⁴ represents a hydrogen atom, a C₁₋₆ alkyl group, an optionally substituted C₃₋₈ cycloalkyl group, an optionally substituted 5- to 14-membered non-aromatic heterocyclic group, an optionally substituted C₆₋₁₄ aromatic hydrocarbon cyclic group or an optionally substituted 5- to 14-membered aromatic heterocyclic group), and the substitutable positions in E are optionally substituted.

28. The compound according to claim 1, a salt thereof or a hydrate thereof, which is represented by the formula (V): 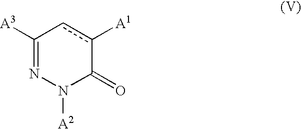
wherein A¹, A², A³ and the partial structure: [symbol]
have the same meanings as defined above, respectively.

29. The compound according to claim 1, a salt thereof or a hydrate thereof, which is represented by the formula (VI): 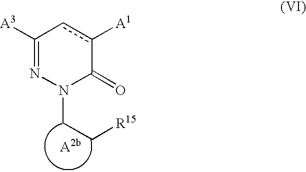
wherein A¹, A³ and the partial structure: [symbol]
have the same meanings as defined above, respectively; the ring A^{2b} represents a C₆₋₈ aromatic hydrocarbon ring or a 5- to 8-membered aromatic heterocyclic ring, each of which is optionally substituted; and R¹⁵ represents a hydroxyl group, a halogen atom, a nitrile group, a C₁₋₆ alkyl group, a C₁₋₆ alkoxy group, a nitro group, an amino group, a C₁₋₆ alkylamino group, a formyl group, a C₁₋₆ alkylcarbonyl group or a trifluoromethyl group.

30. The compound according to claim 28, a salt thereof or a hydrate thereof, wherein A¹, A^{2b} and A³ are independent of each other and each represents a phenyl group, pyrrolyl group, pyridyl group, pyridazinyl group, pyrimidinyl group, pyrazinyl group, thienyl group, thiazolyl group, furyl group, naphthyl group, quinolyl group, isoquinolyl group, benzimidazolyl group, benzothiazolyl group, benzoxazolyl group, imidazopyridyl group, carbazolyl group, cyclopentyl group, cyclohexyl group, cyclohexenyl group, dioxinyl group, adamantyl group, pyrrolidinyl group, piperazinyl group or morpholinyl group, each of which is optionally substituted.

31. The compound according to claim 1, a salt thereof or a hydrate of them, which is represented by the formula: 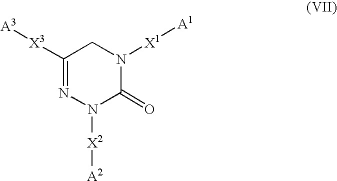
wherein A¹, A², A³, X¹, X² and X³ have the same meanings as defined above, respectively, provided that compounds in the following cases (1) to (4):
(1) the case where X¹ is —NHCO—; each of X² and X³ is a single bond; and each of A¹, A² and A³ is a phenyl group,
(2) the case where each of X¹, X² and X³ is a single bond; and each of A¹, A² and A³is a phenyl group,
(3) the case where each of X¹, X² and X³ is a single bond; A¹ is an o,p-dimethylphenyl group; A² is an o-methylphenyl group; and A³is a phenyl group, and
(4) the case where each of X¹, X² and X³ is a single bond; A¹ is an o-methylphenyl group; A² is a p-methoxyphenyl group; and A³ is a phenyl group are excluded.

32. The compound according to claim 1, a salt thereof or a hydrate of them, which is represented by the formula: 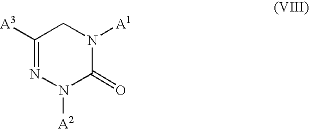
wherein A¹, A² and A³ have the same meanings as defined in the above-mentioned claim 1, respectively, provided that compounds in the following cases (1) to (3):
(1) the case where each of A¹, A² and A³ is a phenyl group,
(2) the case where A¹ is an o,p-dimethylphenyl group; A² is an o-methylphenyl group; and A³is a phenyl group, and
(3) the case where A¹ is an o-methylphenyl group; A² is a p-methoxyphenyl group; and A³ is a phenyl group are excluded.

33. The compound according to claim 32, a salt thereof or a hydrate of them, wherein A¹, A² and A³ are independent of each other and each represents a C₆₋₁₄ aromatic hydrocarbon cyclic group or a 5- to 14-membered aromatic heterocyclic group, each of which is optionally substituted.

34. The compound according to claim 32, a salt thereof or a hydrate of them, wherein A¹, A² and A³ are independent of each other and each represents a phenyl group, pyrrolyl group, pyridyl group, pyridazinyl group, pyrimidinyl group, pyrazinyl group, thienyl group, thiazolyl group, furyl group, naphthyl group, quinolyl group, isoquinolyl group, indolyl group, benzimidazolyl group, benzothiazolyl group, benzoxazolyl group, imidazopyridyl group, carbazolyl group, cyclopentyl group, cyclohexyl group, cyclohexenyl group, dioxinyl group, adamantly group, pyrrolidinyl group, piperidinyl group, piperazinyl group or morpholinyl group, each of which is optionally substituted.

35. The compound according to claim 32, a salt thereof or a hydrate of them, wherein A¹, A² and A³ are independent of each other and each represents a group represented by the formula: 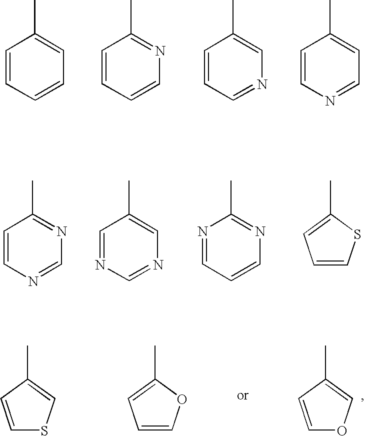
each of which is optionally substituted.

36. The compound according to claim 32, a salt thereof or a hydrate of them, wherein each of A¹, A² and A³ may be substituted with at least one group selected independently from the group consisting of a halogen atom, cyano group, hydroxyl group, amino group, formyl group and nitro group.

37. The compound according to claim 32, a salt thereof or a hydrate of them, wherein the binding positions of substituent groups on A¹, A² and/or A³ are α-positions of the carbon atoms on A¹, A² and/or A³, each of which are bound directly to the triazinone ring.

38. The compound according to claim 1, represented by the following formula, a salt thereof or a hydrate of them: 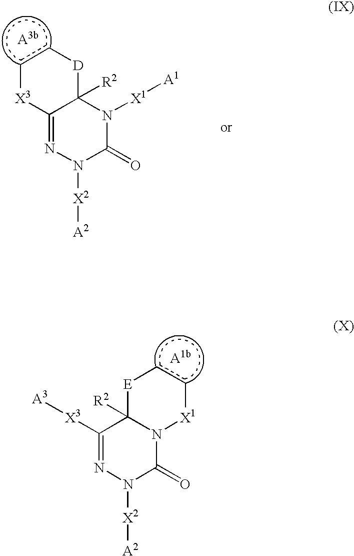
wherein in the formula, A¹, A², A³, A^{1b}, A^{3b}, X¹, X², X³, D, E and R² have the same meanings as defined above, respectively.

39. The compound according to claim 1, a salt thereof or a hydrate thereof, which is any one of the compounds selected from the group consisting of:
2-(2-bromophenyl)-4-(3-methoxyphenyl)-6-(2-pyridyl)-4,5-dihydro-3(2H)-pyridazinone,
2-(2-bromophenyl)-4-(3-hydroxyphenyl)-6-(2-pyridyl)-4,5-dihydro-3(2H)-pyridazinone,
2-(2-bromophenyl)-4-[3-(2-hydroxyethoxy)phenyl]-6-(2-pyridyl)-3(2H)-pyridazinone,
2-(2-cyanophenyl)-4-[3-(2-hydroxyethoxy)phenyl]-6-(2-pyridyl)-3(2H)-pyridazinone,
2-(2-bromophenyl)-6-(2-methoxyphenyl)-4-(2-pyridyl)-3 (2H)-pyridazinone,
4-(4-methoxybenzyl)-6-phenyl-2-(2-tolyl)-3(2H)-pyridazinone,
2,6-diphenyl-4-(a-hydroxy-2-picolyl)-4,5-dihydro-3(2H)-pyridazinone,
2-(2-cyanophenyl)-4-(4-morpholinoethylaminocarbonyl)-6-phenyl-3(2H)-pyridazinone,
2-(2-bromophenyl)-4-(2-methoxyphenyl)-6-(2-pyridyl)-4,5-dihydro-3(2H)-pyridazinone,
2-(2-bromophenyl)-4-(4-methoxyphenyl)-6-(2-pyridyl)-4,5-dihydro-3(2H)-pyridazinone,
2-(2-bromophenyl)-4-(3-bromo-6-methoxyphenyl)-6-(2-pyridyl)-4,5-dihydro-3(2H)-pyridazinone,
2-(2-iodophenyl)-4-(2-methoxyphenyl)-6-(2-pyridyl)-4,5-dihydro-3(2H)-pyridazinone,
4-(2-methoxyphenyl)-2-phenyl-6-(2-pyridyl)-4,5-dihydro-3(2H)-pyridazinone,
2-(2-bromophenyl)-4-phenyl-6-(2-pyridyl)-4,5-dihydro-3(2H)-pyridazinone,
2-(2-bromophenyl)-4-phenyl-6-(3-pyridyl)-4,5-dihydro-3(2H)-pyridazinone,
4,6-diphenyl-2-(2-pyridyl)-4,5-dihydro-3(2H)-pyridazinone,
4-(2-methoxyphenyl)-2-(2-pyridyl)-6-(2-pyridyl)-4,5-dihydro-3(2H)-pyridazinone,
4-(2-cyanophenyl)-2-phenyl-6-(2-pyridyl)-3(2H)-pyridazinone,
2-(2-bromophenyl)-4-(2-methoxyphenyl)-6-(3-pyridyl)-4,5-dihydro-3(2H)-pyridazinone,
4-(2-bromophenyl)-2-phenyl-6-(2-pyridyl)-4,5-dihydro-3(2H)-pyridazinone,
2-(2-methoxyphenyl)-4-phenyl-6-(2-pyridyl)-4,5-dihydro-3(2H)-pyridazinone,
4-phenyl-2-(2-nitrophenyl)-6-(2-pyridyl)-4,5-dihydro-3(2H)-pyridazinone,
2-(2-fluorophenyl)-4-phenyl-6-(2-pyridyl)-4,5-dihydro-3(2H)-pyridazinone,
2-(2-bromophenyl)-4-(2-hydroxyphenyl)-6-(2-pyridyl)-4,5-dihydro-3(2H)-pyridazinone,
2-(2-bromophenyl)-4-(4-hydroxyphenyl)-6-(2-pyridyl)-4,5-dihydro-3(2H)-pyridazinone,
2-(2-bromophenyl)-6-(2-hydroxyphenyl)-4-(2-pyridyl)-3(2H)-pyridazinone,
4-(2-hydroxyphenyl)-2-phenyl-6-(2-pyridyl)-3 (2H)-pyridazinone,
4-(2-hydroxyphenyl)-2-phenyl-6-(2-pyridyl)-4,5-dihydro-3(2H)-pyridazinone,
2-(2-bromophenyl)-4-(2-hydroxyphenyl)-6-(3-pyridyl)-4,5-dihydro-3(2H)-pyridazinone,
2-(2-bromophenyl)-4-(2-dimethylaminoethoxyphenyl)-6-(2-pyridyl)-3(2H)-pyridazinone,
2-(2-bromophenyl)-6-(2-dimethylaminoethoxyphenyl)-4-(2-pyridyl)-3(2H)-pyridazinone,
2-(2-bromophenyl)-4-[3-(2-picolyloxyphenyl)]-6-(2-pyridyl)-3(2)-pyridazinone,
2-phenyl-6-(2-pyridyl)-4-(2-trifluoromethylsulfonyloxyphenyl)-4,5-dihydro-3(2H)-pyridazinone,
2-(2-cyanophenyl)-4-(2-methoxyphenyl)-6-(2-pyridyl)-4,5-dihydro-3(2H)-pyridazinone,
2-(2-cyanophenyl)-4-(2-methoxyphenyl)-6-(2-pyridyl)-3(2H)-pyridazinone,
2-(2-cyanophenyl)-4-(2-hydroxyphenyl)-6-(2-pyridyl)-4,5-dihydro-3(2H)-pyridazinone,
2-(2-cyanophenyl)-4-(2-hydroxyphenyl)-6-(2-pyridyl)-3(2H)-pyridazinone,
2-(2-cyanophenyl)-4-phenyl-6-(2-pyridyl)-3(2H)-pyridazinone,
2-(2-cyanophenyl)-4-(3-bromo-6-methoxyphenyl)-6-(2-pyridyl)-3(2H)-pyridazinone,
2-(2-cyanophenyl)-4-(3-pyridyl)-6-(2-pyridyl)-3(2H)-pyridazinone,
2-(2-cyanophenyl)-4-(2-cyanophenyl)-6-(2-pyridyl)-3(2H)-pyridazinone,
4-(2-bromophenyl)-2-(2-cyanophenyl)-6-(2-pyridyl)-3(2H)-pyridazinone,
2-(2-bromophenyl)-4-(2-hydroxyphenyl)-6-(2-pyridyl)-3(2H)-pyridazinone,
2-(2-bromophenyl)-4-(4-methoxyphenyl)-6-(2-pyridyl)-3(2H)-pyridazinone,
2-phenyl-6-(2-pyridyl)-4-(2-trifluoromethylsulfonyloxyphenyl)-3(2H)-pyridazinone,
2-(2-bromophenyl)-4-phenyl-6-(2-pyridyl)-3 (2H)-pyridazinone,
2-phenyl-4-(3-pyridyl)-6-(2-pyridyl)-3 (2H)-pyridazinone,
4-(2-bromophenyl)-2-phenyl-6-(2-pyridyl)-3(2H)-pyridazinone,
2-(2-bromophenyl)-4-(3-pyridyl)-6-(2-pyridyl)-3(2H)-pyridazinone,
2-(2-chlorophenyl)-4-(4-morpholinoethylaminocarbonyl)-6-phenyl-3(2H)-pyridazinone,
2-(2-nitrophenyl)-4-(3-pyridyl)-6-(2-pyridyl)-3(2H)-pyridazinone,
2-(3-tolyl)-4-(3-pyridyl)-6-(2-pyridyl)-3(2H)-pyridazinone,
2-(4-methanesulfonylphenyl)-4-(3-pyridyl)-6-(2-pyridyl)-3(2H)-pyridazinone,
2-(4-biphenyl)-4-(3-pyridyl)-6-(2-pyridyl)-3(2H)-pyridazinone,
2-(2-naphthyl)-4-(3-pyridyl)-6-(2-pyridyl)-3(2H)-pyridazinone,
2-(3,4-methylenedioxyphenyl)-4-(3-pyridyl)-6-(2-pyridyl)-3(2H)-pyridazinone,
2-(3,4-dichlorophenyl)-4-(3-pyridyl)-6-(2-pyridyl)-3(2H)-pyridazinone,
2-(2-cyanophenyl)-4-phenyl-6-(2-pyrimidyl)-3(2H)-pyridazinone,
2-(2-pyridyl)-4-(2-pyridyl)-6-(2-methoxyphenyl)-3(2H)-pyridazinone,
2-(3-formylphenyl)-4-(3-pyridyl)-6-(2-pyridyl)-3(2H)-pyridazinone,
2-(thiophen-3-yl)-4-(3-pyridyl)-6-(2-pyridyl)-3(2H)-pyridazinone,
2-(3-pyridyl)-4-phenyl-6-(2-pyridyl)-3 (2H)-pyridazinone,
2-(3-pyridyl)-4-phenyl-6-(2-pyrimidyl)-3(2H)-pyridazinone,
2-(2-methoxyphenyl)-4-(3-pyridyl)-6-(2-pyridyl)-4,5-dihydro-3(2H)-pyridazinone,
4-methyl-2,4,6-triphenyl-4,5-dihydro-3(2H)-pyridazinone,
2-(2-bromophenyl)-4-methyl-4,6-diphenyl-4,5-dihydro-3(2H)-pyridazinone,
2-(3-pyridin-1-oxide)-4-phenyl-6-(2-pyridyl)-3(2H)-pyridazinone,
2-(2-cyanopyridin-5-yl)-4-phenyl-6-(2-pyridyl)-3(2H)-pyridazinone,
2-(2-cyanopyridin-3-yl)-4-phenyl-6-(2-pyridyl)-3 (2H)-pyridazinone,
2-(2-cyanopyridin-5-yl)-4-phenyl-6-(2-pyrimidyl)-3(2H)-pyridazinone,
2-(2-cyanopyridin-3-yl)-4-phenyl-6-(2-pyrimidyl)-3(2H)-pyridazinone,
2-(2-cyanophenyl)-4-phenyl-6-(2-pyrazyl)-3(2H)-pyridazinone,
2-(2-cyanophenyl)-4-phenyl-6-(thiazol-2-yl)-3(2H)-pyridazinone, and
2-(2-cyanophenyl)-4-methyl-4,6-diphenyl-4,5-dihydro-3(2H)-pyridazinone.

40. The compound according to claim 1, a salt thereof or a hydrate thereof, said compound is selected from the group consisting of
2-(2-bromophenyl)-4-(2-methoxyphenyl)-6-(2-pyridyl)-4,5-dihydro-3(2H)-pyridazinone,
4-(2-cyanophenyl)-2-phenyl-6-(2-pyridyl)-3(2H)-pyridazinone,
2-(2-bromophenyl)-4-(2-hydroxyphenyl)-6-(2-pyridyl)-4,5-dihydro-3(2H)-pyridazinone,
4-(2-hydroxyphenyl)-2-phenyl-6-(2-pyridyl)-3(2H)- pyridazinone,
2-(2-cyanophenyl)-4-(2-methoxyphenyl)-6-(2-pyridyl)-4,5-dihydro-3(2H)-pyridazinone,
2-(2-cyanophenyl)-4-(2-hydroxyphenyl)-6-(2-pyridyl)-3(2H)-pyridazinone,
2-(2-cyanophenyl)-4-(3-pyridyl)-6-(2-pyridyl)-3 (2H)-pyridazinone,
2-(2-cyanophenyl)-4-(2-cyanophenyl)-6-(2-pyridyl)-3(2H)-pyridazinone,
2-phenyl-4-(3-pyridyl)-6-(2-pyridyl)-3(2H)-pyridazinone, and
2-(2-bromophenyl)-4-(3-pyridyl)-6-(2-pyridyl)-3(2H)-pyridazinone.

41. A pharmaceutical composition comprising:
a pharmacologically effective amount of the compound represented by the formula (I) in claim 1, a salt thereof or a hydrate thereof; and
a pharmaceutically acceptable carrier.

42. A process for treating multiple sclerosis, anxiety or epilepsy, said method comprising:
administering a pharmacologically effective dose of the compound according to claim 1 represented by the formula (I), a salt thereof or a hydrate thereof to a patient in need thereof.

43. A process for treating multiple sclerosis, anxiety or epilepsy, said method comprising:
administering a pharmacologically effective dose of the compound according to claim 19 represented by the formula (II), a salt thereof or a hydrate thereof to a patient in need thereof.

44. A process for treating multiple sclerosis, anxiety or epilepsy, said method comprising:
administering a pharmacologically effective dose of the compound according to claim 28 represented by the formula (V), a salt thereof or a hydrate thereof to a patient in need thereof.

45. A process for treating multiple sclerosis, anxiety or epilepsy, said method comprising:
administering the pharmaceutical composition according to claim 40 to a patient in need thereof.
